(19) United States
(12) Patent Application Publication
(10) Pub. No.: US 2005/0037963 A1
(43) Pub. Date: Feb. 17, 2005

(54) Methods of regulating focal adhesion kinase and its associated cellular functions by fak family-interacting protein

(76) Inventors: Jun-Lin Guan, Ithaca, NY (US); Smita Abbi, South Plainfield, NJ (US)

Correspondence Address: Michael L. Goldman;NIXON PEABODY LLP, Clinton Square, P.O. Box 31051, Rochester, NY, 14603-1051, US

(21) Appl. No.: 10/886,744

(22) Filed: Jul. 8, 2004

Related U.S. Application Data
(60) Provisional application No. 60/486,159, filed on Jul. 10, 2003.

(51) Int. Cl. A61K 38/17

(52) U.S. Cl. 514/12

(57) ABSTRACT

The present invention is directed to treating a subject suffering from a disorder mediated by cell proliferation, such as cancer, by administering a fragment of focal adhesion kinase family kinase-interacting proteins. This method can involve regulating tumor formation or tumor growth in the subject. In addition, the present invention relates to the use of these proteins for regulating G1 to S phase progression of a cell, regulating the expression of p21 in a cell, regulating the phosphorylation of retinoblastoma protein in a cell, regulating retinoblastoma protein/E2F transcription factor 1 complex formation in a cell, regulating detachment-induced apoptosis of a cell, and regulating anchorage-independent growth of a cell.

This application claims the benefit of U.S. Provisional Patent Application Ser. No. 60/486,159, filed Jul. 10, 2003, which is hereby incorporated by reference in its entirety.

The subject matter of this application was made with support from the United States Government under the National Institutes of Health, Grant No. GM48050 and Grant No. GM52890. The U.S. Government may have certain rights.

### FIELD OF THE INVENTION

The present invention relates to methods of regulating focal adhesion kinase (“FAK”) by FAK family-interacting proteins, and uses thereof.

### BACKGROUND OF THE INVENTION

Cancer is a complex and devastating group of diseases that kills one in five adults in developing countries. Although cancers arise from a wide variety of cells and tissues in the body, there are unifying features of this group of diseases. Cancer is predominantly a genetic disease, resulting from the accumulation of mutations that promote clonal selection of cells that exhibit uncontrolled growth and division. For example, by the time a tumor reaches a palpable size of about one centimeter in diameter, it has already undergone about thirty cell doublings, has a mass of approximately one gram, and contains about one billion malignant cells. The result of such uncontrolled growth of tumor cells is the formation of disorganized tissue that compromises the function of normal organs, ultimately threatening the life of the patient. Obviously, methods for prevention, early detection, and effective treatment of cancer are of paramount importance.

The disruption of external or internal regulation of cellular growth leading to uncontrolled cell proliferation can occur at many levels and, indeed, does occur at multiple levels in most tumors. Further, although tumor cells can no longer control their own proliferation, they still must use the same basic cellular machinery employed by normal cells to drive their growth and replication.

Research on the mechanistic basis of carcinogenesis has resulted in a refined understanding of the molecular nature of genetic changes that initiate tumor formation. Specific genes have been identified that are frequently mutated in tumor cells. A few key genes have been identified that are very commonly mutated in a large number of different tumors, such as the oncogene ras and the tumor suppressor genes p53 and Rb. Furthermore, genes that are mutated in tumor cells tend to have functions that cluster in one of the following categories: DNA repair, chromosomal integrity, cell cycle control, growth factor signaling, apoptosis, differentiation, angiogenesis, immune response, and cell migration.

Human breast cancer is a multistep neoplastic process (Beckmann et al., “Multistep Carcinogenesis of Breast Cancer and Tumor Heterogeneity,” J. Mol. Med. 75:429 (1997)), in which integrin signaling plays a significant role, not only in neoplastic transformation (Cance et al., “Immunohistochemical Analyses of Focal Adhesion Kinase Expression in Benign and Malignant Human Breast and Colon Tissues: Correlation with Preinvasive and Invasive Phenotypes,” Clin. Cancer Res. 6:2417 (2000)), but also in invasion of the surrounding stroma and normal mammary gland (Gui et al., “In Vitro Regulation of Human Breast Cancer Cell Adhesion and Invasion Via Integrin Receptors to the Extracellular Matrix,” Br. J. Surg. 82:1192 (1995)), and in metastasis (Gui et al., “Altered Cell-Matrix Contact: A Prerequisite For Breast Cancer Metastasis?” Br. J. Cancer 75:623 (1997)). Recent in vitro and in vivo data implicate the involvement of FAK, one of the major mediators of integrin signaling (Zhao et al., “Role Of Focal Adhesion Kinase In Signaling By The Extracellular Matrix,” Prog. Mol. Subcell Biol. 25:37 (2000)), in breast cancer (Cance et al., “Immunohistochemical Analyses of Focal Adhesion Kinase Expression in Benign and Malignant Human Breast and Colon Tissues: Correlation with Preinvasive and Invasive Phenotypes,” Clin. Cancer Res. 6:2417 (2000); Lin et al., “Progesterone Induces Focal Adhesion in Breast Cancer Cells MDA-MB-231 Transfected with Progesterone Receptor Complementary DNA,” Mol. Endocrinol 14:348 (2000); Beviglia et al., “HGF Induces FAK Activation and Integrin-Mediated Adhesion in MTLn3 Breast Carcinoma Cells,” Int. J. Cancer 83:640 (1999); and Guvakova et al., “The Activated Insulin-Like Growth Factor I Receptor Induces Depolarization in Breast Epithelial Cells Characterized by Actin Filament Disassembly and Tyrosine Dephosphorylation of FAK, Cas, and Paxillin,” Exp. Cell Res. 251:244 (1999)).

FAK is a major mediator of signal transduction by integrins, which has been implicated in the regulation of cell spreading, migration, survival, and proliferation (Clark et al., “Integrins and Signal Transduction Pathways: The Road Taken,” Science 268:233-239 (1995); Schwartz et al., “Integrins: Emerging Paradigms of Signal Transduction,” Annu. Rev. Cell Dev. Biol. 11:549-599 (1995); Parsons, J. T., “Integrin-Mediated Signalling: Regulation by Protein Tyrosine Kinases and Small GTP-Binding Proteins,” Curr. Opin. Cell Biol. 8:146-152 (1996); Cary et al., “Focal Adhesion Kinase in Integrin-Mediated Signaling,” Front. Biosci. 4:D102-D113 (1999); Schlaepfer et al., “Signaling Through Focal Adhesion Kinase,” Prog. Biophys. Mol. Biol. 71:435-478 (1999)). FAK activation and tyrosine phosphorylation have been shown in a variety of cell types to be dependent on integrins binding to their extracellular ligands (Schwartz et al., “Integrins: Emerging Paradigms of Signal Transduction,” Annu. Rev. Cell Dev. Biol. 11:549-599 (1995)). On its activation, FAK is autophosphorylated at Y397, which mediates FAK association with a number of Src homology 2 (“SH2”) domain-containing signaling molecules, including Src family kinases (Chen, H. C. et al., “Association Of Focal Adhesion Kinase With Its Potential Substrate Phosphatidylinositol 3-Kinase,” Proc. Natl. Acad. Sci. USA 91:10148-10152 (1994); Cobb et al., “Stable Association of pp60src and pp59fyn With the Focal Adhesion-Associated Protein Tyrosine Kinase, pp125FAK,” Mol. Cell. Biol. 14:147-155 (1994); Schaller et al., “Autophosphorylation of the Focal Adhesion Kinase, pp125FAK, Directs SH2— Dependent Binding of pp60src,” Mol. Cell Biol. 14:1680 (1994); Xing et al., “Direct Interaction of v-Src With The Focal Adhesion Kinase Mediated by the Src SH2 Domain,” Mol. Biol. Cell 5:413-421 (1994)), p85 subunit of P13K (Chen et al., “Phosphorylation of Tyrosine 397 in Focal Adhesion Kinase is Required for Binding Phosphatidylinositol 3-Kinase,” J. Biol. Chem. 271:26329 (1994)), phospholipase C-γ (Zhang et al., “Focal Adhesion Kinase Promotes Phospholipase C-γ1 Activity,” Proc. Natl. Acad. Sci. USA 96:9021-9026 (1999)), and growth factor receptor-bound protein 7 (“Grb7”) (Han et al., “Association of Focal Adhesion Kinase With Grb7 and Its Role in Cell Migration,” J. Biol. Chem. 274:24425-24430 (1999)). FAK binding to Src family kinases has been proposed to allow phosphorylation of Y925 of FAK by Src, which binds to the SH2 domain of growth factor receptor-bound protein 2 (Schlaepfer et al., “Integrin-Mediated Signal Transduction Linked To Ras Pathway by GRB2 Binding To Focal Adhesion Kinase,” Nature 372:786-791(1994)). The FAK/Src complex formation also leads to tyrosine phosphorylation of a number of other substrates, including paxillin (Burridge et al., “Tyrosine Phosphorylation of Paxillin and Pp125fak Accompanies Cell Adhesion To Extracellular Matrix: A Role in Cytoskeletal Assembly,” J. Cell Biol. 119:893-903 (1992); Schaller et al., “pp125FAK-Dependent Tyrosine Phosphorylation of Paxillin Creates a High-Affinity Binding Site for Crk,” Mol. Cell Biol. 15:2635-2645 (1995)), p130cas (Vuori et al., “Introduction of p130cas Signaling Complex Formation Upon Integrin-Mediated Cell Adhesion: A Role of Src Family Kinases,” Mol. Cell. Biol. 16:2606-2613 (1996); Tachibana et al., “Tyrosine Phosphorylation of Crk-Associated Substrates by Focal Adhesion Kinase: A Putative Mechanism for the Integrin-Mediated Tyrosine Phosphorylation of Crk-Associated Substrates,” J. Biol. Chem. 272:29083-29090 (1997)), and squalene-hopene cyclase (“Shc”) (Schlaepfer et al., “Multiple Grb2-Mediated Integrin-Stimulated Signaling Pathways To ERK2/Mito-Gen-Activated Protein Kinase: Summation of Both C-Src- And Focal Adhesion Kinase-Initiated Tyrosine Phosphorylation Events,” Mol. Cell. Biol. 18:2571-2585 (1998)). Recent studies have shown that Grb7 is phosphorylated by FAK in a Src-independent manner (Han et al., “Role of Grb7 Targeting To Focal Contacts and Its Phosphorylation by Focal Adhesion Kinase in Regulation of Cell Migration,” J. Biol. Chem. 275:28911-28917 (2000)).

FAK and its downstream signaling pathways have been shown to play important roles in the regulation of cell spreading and migration (Ilic et al., “Reduced Cell Motility and Enhanced Focal Adhesion Contact Formation in Cells From FAK-Deficient Mice,” Nature 377:539-544 (1995); Cary et al., “Stimulation of Cell Migration by Overexpression of Focal Adhesion Kinase and Its Association with Src and Fyn,” J. Cell Sci. 109:1787-1794 (1996); Gilmore et al., “Inhibition of Focal Adhesion Kinase (FAK) Signaling in Focal Adhesions Decreases Cell Motility and Proliferation,” Mol. Biol. Cell 7:1209-1224 (1996); Richardson et al., “A Mechanism for Regulation of The Adhesion-Associated Protein Tyrosine Kinase pp125FAK,” Nature 380:538-540 (1996)). FAK−/− fibroblasts derived from FAK-knockout mouse embryo showed a significant decrease in cell migration compared with the cells from wild-type mice (Ilic et al., “Reduced Cell Motility and Enhanced Focal Adhesion Contact Formation in Cells From FAK-Deficient Mice,” Nature 377:539-544 (1995)). Similarly, inhibition of FAK by the FAK C-terminal recombinant protein (i.e., FRNK) caused decreased motility of both fibroblasts and endothelial cells (Gilmore et al., “Inhibition of Focal Adhesion Kinase (FAK) Signaling in Focal Adhesions Decreases Cell Motility and Proliferation,” Mol. Biol. Cell 7:1209-1224 (1996)), as well as a reduced rate of fibroblast spreading (Richardson et al., “A Mechanism for Regulation of the Adhesion-Associated Protein Tyrosine Kinase pp125FAK,” Nature 380:538-540 (1996)). Lastly, overexpression of FAK in a number of cell lines, including the FAK−/− cells, promoted their migration on fibronectins (FN) (Cary et al., “Stimulation of Cell Migration by Overexpression of Focal Adhesion Kinase and Its Association With Src and Fyn,” J. Cell Sci. 109:1787-1794 (1996); Owen et al., “Induced Focal Adhesion Kinase (FAK) Expression In FAK-Null Cells Enhances Cell Spreading and Migration Requiring Both Auto-and Activation Loop Phosphorylation Sites and Inhibits Adhesion-Dependent Tyrosine Phosphorylation of Pyk2,” Mol. Cell Biol. 19:4806-4818 (1999); Sieg et al., “Required Role of Focal Adhesion Kinase (FAK) for Integrin-Stimulated Cell Migration,” J. Cell Sci. 112:2677-2691 (1999)). FAK signaling pathways have also been shown to regulate cell survival and cell cycle progression in integrin-mediated cell adhesion. Overexpression of FAK protected cells from apoptosis induced by cell detachment, serum withdrawal, or other treatments in MDCK cells or primary fibroblasts (Frisch et al., “Control of Adhesion-Dependent Cell Survival by Focal Adhesion Kinase,” J. Cell Biol. 134:793-799 (1996); Ilic et al., “Extracellular Matrix Survival Signals Transduced by Focal Adhesion Kinase Suppress p53-Mediated Apoptosis,” J. Cell Biol. 143:547-560 (1998); and Chan et al., “Suppression of Ultraviolet Irraditation-Induced Apoptosis by Overexpression of Focal Adhesion Kinase in Madin-Darby Canine Kidney Cells,” J. Biol. Chem. 274:26901-26906 (1999)). Conversely, inhibition of FAK by treatment of tumor cell lines with FAK antisense oligonucleotides (Xu et al., “Attenuation of the Expression of the Focal Adhesion Kinase Induces Apoptosis in Tumor Cells,” Cell Growth Differ. 7:413 (1996)), or by microinjection of CEF cells with an anti-FAK monoclonal antibody—(mAb; Hungerford et al., “Inhibition of pp125FAK in Cultured Fibroblasts Results in Apoptosis,” J. Cell Biol. 135:1383-1390 (1996)) induced apoptosis. Microinjection of the C-terminal fragment of FAK into either fibroblasts or endothelial cells inhibited cell cycle progression as measured by bromodeoxyuridine (“BrdU”) incorporation (Gilmore et al., “Inhibition of Focal Adhesion Kinase (FAK) Signaling in Focal Adhesions Decreases Cell Motility and Proliferation,” Mol. Biol. Cell 7:1209-1224 (1996)). Inhibition of FAK tyrosine phosphorylation by disruption of FN matrix assembly also resulted in the delay of the G1 to S transition, suggesting a role for FAK in cell cycle progression (Sechler et al., “Control of Cell Cycle Progression by Fibronectin Matrix Architecture,” J. Biol. Chem. 273:25533-25536 (1998)). Finally, using a tetracycline-regulated expression system, it has been recently shown that expression of wild-type FAK accelerated G1 to S transition, whereas expression of a dominant negative FAK mutant inhibited cell cycle progression at G1 phase (Zhao et al., “Regulation of the Cell Cycle by Focal Adhesion Kinase,” J. Cell Biol. 143:1997-2008 (1998)).

FIP200 (FAK family kinase-interacting protein of 200 kDa) is a protein that has been identified and cloned in yeast two-hybrid screen using Pyk2 N-terminal and kinase domain as a bait (Ueda H. et al., “Suppression of Pyk2 Kinase and Cellular Activities by FIP200,” J. Cell Biol. 149(2):423-430 (2000); Abbi et al., “Regulation of Focal Adhesion Kinase by a Novel Protein Inhibitor FIP200,” Molecular Biology of the Cell 13:3178-3191 (2002)). The 6.6-kb FIP200 cDNA encodes a 1591 aa protein that contains a nuclear localization signal (residues 566-569), a leucine zipper motif (1371-1391), and a coiled-coil structure (1085-1225) (Abbi et al., “Regulation of Focal Adhesion Kinase by a Novel Protein Inhibitor FIP200,” Molecular Biology of the Cell 13:3178-3191 (2002)). FIP200 gene localized in 8q11 chromosome (Chano et al., “Isolation, Characterization and Mapping of the Mouse and Human RB1CC1 Genes,” Gene 291:29-34 (2002)), containing several loci of putative tumor suppressor genes, and loss of heterozygosity (LOH) for this region has been associated with breast cancer (Dahiya et al., “Multiple Sites of Loss of Heterozygosity On Chromosome 8 in Human Breast Cancer Has Differential Correlation With Clinical Parameters,” Int. J. Oncology 12:811-816 (1998)).

Results suggest that endogenous and exogenously expressed epitope tagged FIP200 are localized both in the nucleus and cytoplasm of fibroblasts and several breast cancer cell lines (Ueda et al., “Suppression of Pyk2 Kinase and Cellular Activities by FIP200,” J. Cell Biol. 149(2):423-430 (2000); Abbi et al., “Regulation of Focal Adhesion Kinase by a Novel Protein Inhibitor FIP200,” Molecular Biology of the Cell 13:3178-3191 (2002)). Earlier studies demonstrated that FIP200 directly interacts with both FAK (Ueda et al., “Suppression of Pyk2 Kinase and Cellular Activities by FIP200,” J. Cell Biol. 149(2):423-430 (2000)) and Pyk2 (proline-rich tyrosine kinase 2) (Abbi et al., “Regulation of Focal Adhesion Kinase by a Novel Protein Inhibitor FIP200,” Molecular Biology of the Cell 13:3178-3191 (2002)) and inhibits their kinase activity. In addition, it has been shown that exogenous expression of FIP200 inhibits cell spreading, migration and cell cycle progression in fibroblast model (Abbi et al., “Regulation of Focal Adhesion Kinase by a Novel Protein Inhibitor FIP200,” Molecular Biology of the Cell 13:3178-3191 (2002)).

FIP200 has been independently cloned by differential display analysis of the multi-drug resistant osteosarcoma cell line (Chano et al., “Identification of RB1CC1, a Novel Human Gene That Can Induce RB1 in Various Human Cells,” Oncogene 21:1295-1298 (2002)). They found close correlation between expression levels of FIP200 and pRb in various cancer cell lines and normal human tissues and suggested that FIP200 may be an important regulator of pRb (Chano et al., “Identification of RB1CC1, a Novel Human Gene That Can Induce RB1 in Various Human Cells,” Oncogene 21:1295-1298 (2002)). Furthermore, it has been demonstrated that FIP200 and pRb genes are preferentially co-expressed and contributed to the maturation of human embryonic musculoskeletal cells, and may regulate the proliferative activity and maturation of tumor cells derived from these tissues (Chano et al., “Preferential Expression of RB1-Inducible Coiled-Coil 1 in Terminal Differentiated Musculoskeletal Cells,” American Journal of Pathology 161:359-364 (2002)). Finally, 20% of primary breast cancers that they screened contained deletion mutations in FIP200, predicted to yield a truncated protein (Chano et al., “Truncating Mutations of RB1CC1 in Human Breast Cancer,” Nature Genetics 31:285-288 (2002)).

In contrast to rapid progress in elucidating the FAK downstream signaling pathways, relatively little is known about the mechanisms of regulation of FAK activity and its associated cellular functions.

The present invention is directed to overcoming these and other deficiencies in the art.

### SUMMARY OF THE INVENTION

The present invention relates to a method of treating a subject suffering from a disorder mediated by cell proliferation. This method involves administering a therapeutically effective amount of a polypeptide or protein having an amino acid sequence of SEQ ID NO:3, SEQ ID NO:5, or SEQ ID NO:7 to the subject under conditions effective to inhibit the cell proliferation disorder.

The present invention also relates to a method of regulating activity of a kinase. This method involves contacting the kinase with a polypeptide or protein having an amino acid sequence of SEQ ID NO:3, SEQ ID NO:5, or SEQ ID NO:7 under conditions effective to regulate the activity of the kinase.

Another aspect of the present invention is an expression vector including transcriptional and translational regulatory nucleotide sequences operably linked to a nucleic acid molecule having a nucleotide sequence of SEQ ID NO:4, SEQ ID NO:6, or SEQ ID NO:8.

Yet another aspect of the present invention is a method of regulating G1 to S phase progression of a cell. This method involves contacting the cell with a polypeptide or protein having an amino acid sequence comprising SEQ ID NO:3, SEQ ID NO:5, or SEQ ID NO:7 under conditions effective to regulate G1 to S phase progression of the cell.

The present invention also relates to a method of regulating expression of p21 in a cell. This method involves contacting the cell with a polypeptide or protein having an amino acid sequence comprising SEQ ID NO:3, SEQ ID NO:5, or SEQ ID NO:7 under conditions effective to regulate expression of p21.

Another aspect of the present invention is a method of regulating phosphorylation of retinoblastoma protein in a cell. This method involves contacting the cell with a polypeptide or protein having an amino acid sequence comprising SEQ ID NO:3, SEQ ID NO:5, or SEQ ID NO:7 under conditions effective to regulate phosphorylation of retinoblastoma protein.

Yet another aspect of the present invention is a method of regulating retinoblastoma protein/E2F transcription factor 1 complex formation in a cell. This method involves contacting the cell with a polypeptide or protein having an amino acid sequence comprising SEQ ID NO:3, SEQ ID NO:5, or SEQ ID NO:7 under conditions effective to regulate retinoblastoma protein/E2F transcription factor 1 complex formation.

The present invention also relates to a method of regulating detachment-induced apoptosis of a cell. This method involves contacting the cell with a protein or polypeptide having an amino acid sequence comprising SEQ ID NO:3, SEQ ID NO:5, or SEQ ID NO:7 under conditions effective to regulate detachment-induced apoptosis of the cell.

Another aspect of the present invention is a method of regulating anchorage-independent growth of a cell. This method involves contacting the cell with a protein or polypeptide having an amino acid sequence comprising SEQ ID NO:3, SEQ ID NO:5, or SEQ ID NO:7 under conditions effective to regulate anchorage-independent growth of the cell.

Yet another aspect of the present invention is a method of regulating tumor formation in a subject. This method involves administering a therapeutically effective amount of a protein or polypeptide having an amino acid sequence comprising SEQ ID NO:3, SEQ ID NO:5, or SEQ ID NO:7 to the subject under conditions effective to regulate tumor formation.

The present invention also relates to a method of regulating tumor growth in a subject. This method involves administering a therapeutically effective amount of a protein or polypeptide having an amino acid sequence comprising SEQ ID NO:3, SEQ ID NO:5, or SEQ ID NO:7 to the subject under conditions effective to regulate tumor growth.

Another aspect of the present invention is a method of regulating tumor formation in a subject. This method involves administering a therapeutically effective amount of a nucleic acid molecule having a nucleotide sequence comprising SEQ ID NO:4, SEQ ID NO:6, or SEQ ID NO:8 to the subject under conditions effective to regulate tumor formation.

Yet another aspect of the present invention is a method of regulating tumor growth in a subject. This method involves administering a therapeutically effective amount of a nucleic acid molecule having a nucleotide sequence comprising SEQ ID NO:4, SEQ ID NO:6, or SEQ ID NO:8 to the subject under conditions effective to regulate tumor growth.

The methods disclosed in the present invention may be used to regulate fundamental cellular functions, such as cell migration, cell proliferation, and cell cycle progression. This may provide potential new therapeutics for cancer and other diseases that are mediated by these cellular functions.

### BRIEF DESCRIPTION OF THE DRAWINGS

FIGS. 1A-D are western blots showing the association and localization of FIP200 and FAK. Lysates were prepared from MDA-MB231 breast carcinoma cells that had been suspended, or replated on FN, type IV collagen, or type I collagen, as indicated. Lysates were immunoprecipitated by anti-FIP200N and analyzed by western blotting with anti-FAK, FIG. 1A, or anti-FIP200C, FIG. 1B. The whole cell lysates (WCL) were also analyzed directly by western blotting with anti-pFAKY397, FIG. 1C, or anti-FAK, FIG. 1D.

FIG. 1E is an immunofluorescense assay showing the colocalization of FIP200 and FAK in peripheral focal contacts. NIH 3T3 cells (top panels) or ECV304 cells transfected with FIP200 (bottom panels) were processed for immunofluorescence using anti-FIP200N or anti-FAK. Examples of colocalization of FIP200 and FAK in peripheral focal contacts are marked by arrows.

FIG. 2A is a schematic of the FIP200 protein and its fragments. Full-length FIP200 is shown on top. NT-FIP, CT-FIP, and MD-FIP segments of FIP200 are shown below.

FIGS. 2B-D are western blots showing an analysis of FIP200 association with FAK. FIG. 2B shows 293T cells transfected with an expression vector encoding HA-FAK and vectors encoding Flag-FIP200, its segments or empty vector control (V), as indicated. Lysates were immunoprecipitated with anti-Flag followed by western blotting with anti-FAK to detect associated HA-FAK (top panel) or with anti-Flag to verify similar amounts of samples in the immunoprecipitates (bottom panel; Flag-FIP200 and segments are marked by arrowheads). FIG. 2C shows 293T cells transfected with vectors encoding HA-FAK (WT) or its fragments (N-terminal, kinase domain and C-terminal) or with kinase domain of Pyk2, as indicated. Lysates from the transfected cells were then incubated with immobilized GST fusion proteins containing FIP200 segments or GST alone, as indicated. The bound proteins were resolved on SDS-PAGE and were analyzed by western blotting with mAb 12CA5 (anti-HA). FIG. 2D shows equal amounts of immobilized GST-fusion proteins containing FIP200 fragments, or GST alone, incubated with 1 μg of recombinant FAK. The bound proteins were resolved on SDS-PAGE and were analyzed by western blotting with anti-FKA (top panel). The membrane was also stained with Ponceau S stain to detect GST-fusion proteins (marked with arrowheads; bottom panel).

FIGS. 3A-B are a schematic and western blot showing the inhibition of FAK activity by FIP200. FIG. 3A shows the kinase activity of FAK assayed in the presence of various amounts of GST fusion proteins containing FIP200 segments, or GST alone, as indicated. Relative kinase activities were normalized to FAK activity in the absence of GST fusion protein. The mean±SE of relative kinase activities from three independent experiments are shown. The inset shows Coomassie blue staining of a representation preparation of the fusion proteins (1, GST-NT-FIP; 2, GST-MD-FIP; and 3, GST-CT-FIP). FIG. 3B shows 293T cells cotransfected with plasmid encoding HA-FAK and HA-FIP200, its segments, or vector control, as indicated. One day after transfection, cells were trypsinized and either kept in suspension (S) or replated on FN (10 μg/ml) for 30 min. They were then lysed and immunoprecipitated with anti-HA followed by western blotting with PY20 to detect FAK phosphorylation (top panel) or anti-HA to verify similar FAK expression levels (middle panel). The corresponding whole cell lysates (WCL) were resolved on a SDS-PAGE gel and were western blotted with anti-HA to detect similar amounts of FIP200 and its fragments (marked by arrowheads, bottom panel).

FIGS. 4A-D are western blots showing the effects of FIP200 on FAK downstream signaling. 293 cells were cotransfected with plasmids encoding FAK, NT-FIP, or empty vector as controls, along with vectors encoding GFP-paxillin (FIG. 4A), Myc-p130cas (FIG. 4B), HA-Shc (FIG. 4C), or HA-Grb7 (FIG. 4D), as indicated. One day after transfection, cells were trypsinized and replated on FN (10 μg/ml) for 30 min. Whole cell lysates were then immunoprecipitated with anti-GFP, anti-Myc, or anti-HA to pull-down epitope-tagged paxillin, p130cs, Shc, and Grb7, respectively. The immune complexes were then analyzed by western blotting with PY20 to detect their phosphorylation (top panels), or with antipaxillin, anti-Myc, or anti-HA to show their respective expression levels (bottom panels). The position of HA-Shc is indicated by arrows in FIG. 4C.

FIGS. 5A-D are diagrams showing the inhibition of cell spreading by FIP200. FIG. 5A shows NIH3T3 cells transfected with expression vectors encoding FIP200 or CT-FIP, as indicated. One day after transfection, cells were trypsinized and replated on FN (10 μg/ml) for 45 min. Cells were fixed and processed for immunostaining with anti-HA to detect the positively transfected cells (green) and with antivinculin to visualize all cells (red). FIGS. 5B-D show NIH3T3 cells transfected with vectors encoding FIP200, its segments, or empty vector control, and plasmid encoding a HA-tagged FAK (in some experiments), along with a plasmid encoding β-Gal, as indicated. One day after transfection, cells were trypsinized and replated on FN (10 μg/ml) for 45 min. The cells were then fixed, and β-Gal assays were performed to identify the positively transfected cells. The mean±SE of percentage of spread cells from three independent experiments are shown in FIG. 5B. Aliquots of whole cell lysates (WCL) were also analyzed directly by western blotting using anti-Flag to detect FIP200 and its fragments, FIG. 5C, or anti-HA to detect FAK, FIG. 5D.

FIGS. 6A-C are diagrams showing the inhibition of cell migration by FIP200. FIGS. 6A-B show NIH3T3 cells grown on FN (10 μg/ml) transfected with FIP200, its segments, or empty vector control, with vectors encoding FAK or paxillin in some experiments, along with a plasmid encoding GFP in 7:1 ratio, as indicated. One day after transfection, the cell monolayer was wounded with a p10 tip, incubated at 37° C., and images were captured at 2-h intervals until 8 h. Images from representative experiments are shown in FIG. 6A. The rate of migration was measured by quantifying the total distance that the positively transfected cells (GFP⁺) moved from the edge of the wound toward the center of the wound in 8 h, FIG. 6B, mean±SE of the rate of migration from three independent experiments (*p=0.48, 0,76, 0.32, and 0.89 for samples from left to right, in comparison with vector alone transfected cells). FIG. 6C shows the motility of cells on FN (5 μg/ml) assayed using OMAware based on time-lapse video microscopy. Representative field of cell tracks of control untransfected cells (panel a), cells transfected with expression vector encoding FIP200 (panel b), and cells transfected with both vectors encoding FIP200 and FAK (panel c) are shown. The arrows denote positively transfected cells. The untransfected cells in the same field serve as internal controls.

FIGS. 7A-C are schematics showing the regulation of cell cycle progression by FIP200. FIGS. 7A-B show NIH3T3 cells transfected with expression vectors encoding HA-FIP200 or its segments, or an irrelevant control protein (C) or were mock transfected, as indicated. They were then analyzed for BrdU incorporation. FIG. 7A shows representative fields for cells transfected with HA-FIP200 or the control. Immunostaining with anti-HA identifies positively transfected cells (green), and staining with anti-BrdU shows cells with new DNA synthesis (red). FIG. 7B shows the mean±SE of three independent experiments of the percentage of BrdU⁺/positively transfected cells as determined by analyzing at least 80 positively transfected cells for each transfection in multiple fields. FIG. 7C shows NIH3T3 cells cotransfected with an expression vector encoding FAK and that encoding HA-FIP200 or empty vector control (V), as indicated. A plasmid encoding β-Gal was also included. Cells were then analyzed for BrdU incorporation. The positively transfected cells were identified by immunostaining with anti-β-Gal. The percentage of BrdU⁺ β-Gal⁺ cells was determined by analyzing 40-50 β-Gal⁺cells for each transfection in multiple fields. The percentage of BrdU-positive cells was normalized to the vector control of 100%. The results show mean±SE of three independent experiments (*p=0.18 and 0.59 are values for empty vector plus FAK transfection and HA-FIP200 plus FAK transfection, respectively, in comparison with value from empty vector alone transfection). Inset shows similar expression levels of FIP200 (α-HA blot) with or without cotransfection of FAK (KT3 blot).

FIGS. 8A-C are western blots and a schematic showing the disruption of endogenous FIP200 interaction with FAK. FIGS. 8A-B show NIH3T3 cells cotransfected with plasmid encoding HA-tagged KD^{KR} or an irrelevant control protein (Grb7 SH2 domain, designated as C) and plasmids encoding HA-FAK or HA-Pyk2, as indicated. One day after transfection, cells were trypsinized and replated on PLL (0.1 mg/ml) or FN (10 μg/ml, FIG. 8A) or serum starved and treated with or without sorbitol (400 mM, 10 min, FIG. 8B), as indicated. Cell lysates were then immunoprecipitated with anti-HA or anti-Pyk2 and were western blotted with PY20 (top panel) or anti-HA (middle panel). Whole cell lysates (WCL) were also blotted with anti-HA (bottom panel) to show levels of transfected KD^{KR} and control protein, as shown in FIG. 8C. FIG. 8C shows NIH3T3 cells transfected with plasmid encoding HA-tagged KD^{KR} or an irrelevant control protein, or mock transfected, as indicated. They were then analyzed for BrdU incorporation on both FN (10 μg/ml) and PLL (0.1 mg/ml). The percentage of BrdU⁺/positively transfected cells was calculated and normalized to that of untransfected cells in each experiment. The mean±SE are shown for data from three independent experiments.

FIG. 9 is a schematic showing a working hypothesis of FIP200 interaction with FAK during cell adhesion.

FIG. 10 is a schematic showing the inhibition of G1 to S phase progression by FIP200 in human breast cancer cells. The sub-confluent cultures of U20S, MCF-7, and MDA-231 cells were transfected with vectors encoding HA-FIP200 or irrelevant control protein (Grb7). 24 hours post-transfection, cells were re-plated on glass cover-slips in 10% FBS and 150 uM BrdU. 16 hours later, cellular DNA was digested with DNase I and cells were processed for the double immunofluorescence staining with anti-HA (to detect transfected cells) and anti-BrdU (to detect cells that progressed to S phase and incorporated BrdU) Abs. At least 200 positively transfected cells were counted. Data represent % of BrdU+/transfected cells of the total number of transfected cells counted. Percent of BrdU incorporation in Grb7 and mock transfected cells were similar (see FIG. 11).

FIGS. 11A-C are schematics showing the inhibition of G1 to S phase progression by several FIP200 deletion mutants. Sub-confluent cultures of MCF-7 cells (FIG. 11A), or FAK null (FIG. 11B), mouse embryo fibroblasts were transfected with expression vectors encoding HA-FIP200 or its deletion mutants (the structures are shown in FIG. 11C) or an irrelevant control protein (Grb7). BrdU incorporation assay was conducted in an identical manner as described for FIG. 10.

FIG. 12 is a western blot showing the effect of FIP200 and various fragments on p21 levels and pRb/E2 μl complex formation. Sub-confluent MCF-7 cells were co-transfected with 2 μg of plasmids encoding HA-FIP200, HA-N1a, or an empty vector (V). 24 hours post-transfection, cells were re-plated in 10% FBS. 24 hours later, cells were lysed and immunoprecipitated with anti-E2F1 Abs or mouse IgG (control). Effects of FIP200 and N1a on pRb/E2F1 complex formation were assayed by immunoblotting with anti-pRb Abs. Whole cell lysates were blotted with anti-HA Abs to detect exogenously expressed FIP200 and N1a levels or with anti-p21 Abs to detect the changes in p21 levels by FIP200 and N1a.

### DETAILED DESCRIPTION OF THE INVENTION

The present invention relates to a method of treating a subject suffering from a disorder that is mediated by cell proliferation. This method involves administering a therapeutically effective amount of a polypeptide or protein having an amino acid sequence of SEQ ID NO:3, SEQ ID NO:5, or SEQ ID NO:7 to the subject under conditions effective to treat the cell proliferation disorder.

Many disease conditions, particularly cancers, have been associated with abnormal cellular functions. These functions include integrin-mediated cell adhesion, cell migration, cell proliferation, cell cycle progression, and cell spreading. Such abnormal cellular functions may be associated with one or more disease conditions. The proteins or polypeptides described herein are useful in regulating such cellular functions as integrin-mediated cell adhesion, cell migration, cell proliferation, cell cycle progression, and cell spreading. Thus, this method is suitable for the treatment of cancerous conditions, including breast cancer, colon cancer, central nervous system cancer, leukemia, melanoma, lung cancer, ovarian cancer, prostate cancer, and renal cancer, and other diseases, including, but not limited to, hypertension, hypotension, ischemia, inflammation, arthritis, diabetic retinopathy, myocardial infarction, and cardio-vascular disease.

This method involves administering a suitable protein or polypeptide described herein to a diseased subject under conditions to allow inhibition or retardation of abnormal cell proliferation. In this and all aspects of the present invention that involve treatment of a disease condition in a subject, suitable subjects include, but are not limited to, mammals, including humans.

The FAK family kinase-interacting protein of 200 kDa (“FIP200”) has an amino acid sequence corresponding to SEQ ID NO:1, as follows:

```
Met Lys Leu Tyr Val Phe Leu Val Asn Thr Gly Thr Thr Leu Thr Phe
    1 5 10 15
Asp Thr Glu Leu Thr Val Gln Thr Val Ala Asp Leu Lys His Ala Ile
                          20 25 30
Gln Ser Lys Tyr Lys Ile Ala Ile Gln His Gln Val Leu Val Val Asn
                  35 40 45
Gly Gly Glu Cys Met Ala Ala Asp Arg Arg Val Cys Thr Tyr Ser Ala
          50 55 60
Gly Thr Asp Thr Asn Pro Ile Phe Leu Phe Asn Lys Glu Met Ile Leu
  65 70 75 80
Cys Asp Arg Pro Pro Ala Ile Pro Lys Thr Thr Phe Ser Thr Glu Asn
                                  85 90 95
Asp Met Glu Ile Lys Val Glu Glu Ser Leu Met Met Pro Ala Val Phe
                        100 105 110
His Thr Val Ala Ser Arg Thr Gln Leu Ala Leu Glu Met Tyr Glu Val
                115 120 125
Ala Lys Lys Leu Cys Ser Phe Cys Glu Gly Leu Val His Asp Glu His
        130 135 140
Leu Gln His Gln Gly Trp Ala Ala Ile Met Ala Asn Leu Glu Asp Cys
145 150 155 160
Ser Asn Ser Tyr Gln Lys Leu Leu Phe Lys Phe Glu Ser Ile Tyr Ser
                                165 170 175
Asn Tyr Leu Gln Ser Ile Glu Asp Ile Lys Leu Lys Leu Thr His Leu
                        180 185 190
Gly Thr Ala Val Ser Val Met Ala Lys Ile Pro Leu Leu Glu Cys Leu
                195 200 205
Thr Arg His Ser Tyr Arg Glu Cys Leu Gly Arg Leu Asp Ser Leu Pro
        210 215 220
Glu His Glu Asp Ser Glu Lys Ala Glu Thr Lys Arg Ser Thr Glu Leu
225 230 235 240
Val Leu Ser Pro Asp Met Pro Arg Thr Thr Asn Glu Ser Leu Leu Thr
                                245 250 255
Ser Phe Pro Lys Ser Val Glu His Val Ser Pro Asp Thr Ala Asp Ala
                        260 265 270
Glu Ser Gly Lys Glu Ile Arg Glu Ser Cys Gln Ser Thr Val His Gln
                275 280 285
Gln Asp Glu Thr Thr Ile Asp Thr Lys Asp Gly Asp Leu Pro Phe Phe
        290 295 300
Asn Val Ser Leu Leu Asp Trp Ile Asn Val Gln Asp Arg Pro Asn Asp
305 310 315 320
Val Glu Ser Leu Val Arg Lys Cys Phe Asp Ser Met Ser Arg Leu Asp
                                325 330 335
Pro Arg Ile Ile Arg Pro Phe Ile Ala Glu Cys Arg Gln Thr Ile Ala
                        340 345 350
Lys Leu Asp Asn Gln Asn Met Lys Ala Ile Lys Gly Leu Glu Asp Arg
                355 360 365
Leu Tyr Ala Leu Asp Gln Met Ile Ala Ser Cys Gly Arg Leu Val Asn
        370 375 380
Glu Gln Lys Glu Leu Ala Gln Gly Phe Leu Ala Asn Gln Lys Arg Ala
385 390 395 400
Glu Asn Leu Lys Asp Ala Ser Val Leu Pro Asp Leu Cys Leu Ser His
                                405 410 415
Ala Asn Gln Leu Met Ile Met Leu Gln Asn His Arg Lys Leu Leu Asp
                        420 425 430
Ile Lys Gln Lys Cys Thr Thr Ala Lys Gln Glu Leu Ala Asn Asn Leu
                435 440 445
His Val Arg Leu Lys Trp Cys Cys Phe Val Met Leu His Ala Asp Gln
        450 455 460
Asp Gly Glu Lys Leu Gln Ala Leu Leu Arg Leu Val Ile Glu Leu Leu
465 470 475 480
Glu Arg Val Lys Ile Val Glu Ala Leu Ser Thr Val Pro Gln Met Tyr
                                485 490 495
Cys Leu Ala Val Val Glu Val Val Arg Arg Lys Met Phe Ile Lys His
                        500 505 510
Tyr Arg Glu Trp Ala Gly Ala Leu Val Lys Asp Gly Lys Arg Leu Tyr
                515 520 525
Glu Ala Glu Lys Ser Lys Arg Glu Ser Phe Gly Lys Leu Phe Arg Lys
        530 535 540
Ser Phe Leu Arg Asn Arg Leu Phe Arg Gly Leu Asp Ser Trp Pro Pro
545 550 555 560
Ser Phe Cys Thr Gln Lys Pro Arg Lys Phe Asp Cys Glu Leu Pro Asp
                                565 570 575
Ile Ser Leu Lys Asp Leu Gln Phe Leu Gln Ser Phe Cys Pro Ser Glu
                        580 585 590
Val Gln Pro Phe Leu Arg Val Pro Leu Leu Cys Asp Phe Glu Pro Leu
                595 600 605
His Gln His Val Leu Ala Leu His Asn Leu Val Lys Ala Ala Gln Ser
        610 615 620
Leu Asp Glu Met Ser Gln Thr Ile Thr Asp Leu Leu Ser Glu Gln Lys
625 630 635 640
Ala Ser Val Ser Gln Thr Ser Pro Gln Ser Ala Ser Ser Pro Arg Met
                                645 650 655
Glu Ser Thr Ala Gly Ile Thr Thr Thr Thr Ser Pro Arg Thr Pro Pro
                        660 665 670
Pro Leu Thr Val Gln Asp Pro Leu Cys Pro Ala Val Cys Pro Leu Glu
                675 680 685
Glu Leu Ser Pro Asp Ser Ile Asp Ala His Thr Phe Asp Phe Glu Thr
        690 695 700
Ile Pro His Pro Asn Ile Glu Gln Thr Ile His Gln Val Ser Leu Asp
705 710 715 720
Leu Asp Ser Leu Ala Glu Ser Pro Glu Ser Asp Phe Met Ser Ala Val
                                725 730 735
Asn Glu Phe Val Ile Glu Glu Asn Leu Ser Ser Pro Asn Pro Ile Ser
                        740 745 750
Asp Pro Gln Ser Pro Glu Met Met Val Glu Ser Leu Tyr Ser Ser Val
                755 760 765
Ile Asn Ala Ile Asp Ser Arg Arg Met Gln Asp Thr Asn Val Cys Gly
        770 775 780
Lys Glu Asp Phe Gly Asp His Thr Ser Leu Asn Val Gln Leu Glu Arg
785 790 795 800
Cys Arg Val Val Ala Gln Asp Ser His Phe Ser Ile Gln Thr Ile Lys
                                805 810 815
Glu Asp Leu Cys His Phe Arg Thr Phe Val Gln Lys Glu Gln Cys Asp
                        820 825 830
Phe Ser Asn Ser Leu Lys Cys Thr Ala Val Glu Ile Arg Asn Ile Ile
                835 840 845
Glu Lys Val Lys Cys Ser Leu Glu Ile Thr Leu Lys Glu Lys His Gln
        850 855 860
Lys Glu Leu Leu Ser Leu Lys Asn Glu Tyr Glu Gly Lys Leu Asp Gly
865 870 875 880
Leu Ile Lys Glu Thr Glu Glu Asn Glu Asn Lys Ile Lys Lys Leu Lys
                                885 890 895
Gly Glu Leu Val Cys Leu Glu Glu Val Leu Gln Asn Lys Asp Asn Glu
                        900 905 910
Phe Ala Leu Val Lys His Glu Lys Glu Ala Val Ile Cys Leu Gln Asn
                915 920 925
Glu Lys Asp Gln Lys Leu Leu Glu Met Glu Asn Ile Met His Ser Gln
        930 935 940
Asn Cys Glu Ile Lys Glu Leu Lys Gln Ser Arg Glu Ile Val Leu Glu
945 950 955 960
Asp Leu Lys Lys Leu His Val Glu Asn Asp Glu Lys Leu Gln Leu Leu
                                965 970 975
Arg Ala Glu Leu Gln Ser Leu Glu Gln Ser His Leu Lys Glu Leu Glu
                        980 985 990
Asp Thr Leu Gln Val Arg His Ile Gln Glu Phe Glu Lys Val Met Thr
                995 1000 1005
Asp His Arg Val Ser Leu Glu Glu Leu Lys Lys Glu Asn Gln Gln Ile
      1010 1015 1020
Ile Asn Gln Ile Gln Glu Ser His Ala Glu Ile Ile Gln Glu Lys Glu
1025 1030 1035 1040
Lys Gln Leu Gln Glu Leu Lys Leu Lys Val Ser Asp Leu Ser Asp Thr
                              1045 1050 1055
Arg Cys Lys Leu Glu Val Glu Leu Ala Leu Lys Glu Ala Glu Thr Asp
                      1060 1065 1070
Glu Ile Lys Ile Leu Leu Glu Glu Ser Arg Ala Gln Gln Lys Glu Thr
              1075 1080 1085
Leu Lys Ser Leu Leu Glu Gln Glu Thr Glu Asn Leu Arg Thr Glu Ile
      1090 1095 1100
Ser Lys Leu Asn Gln Lys Ile Gln Asp Asn Asn Glu Asn Tyr Gln Val
1105 1110 1115 1120
Gly Leu Ala Glu Leu Arg Thr Leu Met Thr Ile Glu Lys Asp Gln Cys
                      1125 1130 1135
Ile Ser Glu Leu Ile Ser Arg His Glu Glu Glu Ser Asn Ile Leu Lys
              1140 1145 1150
Ala Glu Leu Asn Lys Val Thr Ser Leu His Asn Gln Ala Phe Glu Ile
      1155 1160 1165
Glu Lys Asn Leu Lys Glu Gln Ile Ile Glu Leu Gln Ser Lys Leu Asp
      1170 1175 1180
Ser Glu Leu Ser Ala Leu Glu Arg Gln Lys Asp Glu Lys Ile Thr Gln
1185 1190 1195 1200
Gln Glu Glu Lys Tyr Glu Ala Ile Ile Gln Asn Leu Glu Lys Asp Arg
                              1205 1210 1215
Gln Lys Leu Val Ser Ser Gln Glu Gln Asp Arg Glu Gln Leu Ile Gln
                      1220 1225 1230
Lys Leu Asn Cys Glu Lys Asp Glu Ala Ile Gln Thr Ala Leu Lys Glu
              1235 1240 1245
Phe Lys Leu Glu Arg Glu Val Val Glu Lys Glu Leu Leu Glu Lys Val
      1250 1255 1260
Lys His Leu Glu Asn Gln Ile Ala Lys Ser Pro Ala Ile Asp Ser Thr
1265 1270 1275 1280
Arg Gly Asp Ser Ser Ser Leu Val Ala Glu Leu Gln Glu Lys Leu Gln
                              1285 1290 1295
Glu Glu Lys Ala Lys Phe Leu Glu Gln Leu Glu Glu Gln Glu Lys Arg
                      1300 1305 1310
Lys Asn Glu Glu Met Gln Asn Val Arg Thr Ser Leu Ile Ala Glu Gln
              1315 1320 1325
Gln Thr Asn Phe Asn Thr Val Leu Thr Arg Glu Lys Met Arg Lys Glu
      1330 1335 1340
Asn Ile Ile Asn Asp Leu Ser Asp Lys Leu Lys Ser Thr Met Gln Gln
1345 1350 1355 1360
Gln Glu Arg Asp Lys Asp Leu Ile Glu Ser Leu Ser Glu Asp Arg Ala
                              1365 1370 1375
Arg Leu Leu Glu Glu Lys Lys Lys Leu Glu Glu Glu Val Ser Lys Leu
                      1380 1385 1390
Arg Ser Ser Ser Phe Val Pro Ser Pro Tyr Val Ala Thr Ala Pro Glu
              1395 1400 1405
Leu Tyr Gly Ala Cys Ala Pro Glu Leu Pro Gly Glu Ser Asp Arg Ser
      1410 1415 1420
Ala Val Glu Thr Ala Asp Glu Gly Arg Val Asp Ser Ala Met Glu Thr
1425 1430 1435 1440
Ser Met Met Ser Val Gln Glu Asn Ile His Met Leu Ser Glu Glu Lys
                              1445 1450 1455
Gln Arg Ile Met Leu Leu Glu Arg Thr Leu Gln Leu Lys Glu Glu Glu
                      1460 1465 1470
Asn Lys Arg Leu Asn Gln Arg Leu Met Ser Gln Ser Met Ser Ser Val
              1475 1480 1485
Ser Ser Arg His Ser Glu Lys Ile Ala Ile Arg Asp Phe Gln Val Gly
      1490 1495 1500
Asp Leu Val Leu Ile Ile Leu Asp Glu Arg His Asp Asn Tyr Val Leu
1505 1510 1515 1520
Phe Thr Val Ser Pro Thr Leu Tyr Phe Leu His Ser Glu Ser Leu Pro
                              1525 1530 1535
Ala Leu Asp Leu Lys Pro Ala Ser Gly Ala Ser Arg Arg Pro Trp Val
                      1540 1545 1550
Leu Gly Lys Val Met Glu Lys Glu Tyr Cys Gln Ala Lys Lys Ala Gln
              1555 1560 1565
Asn Arg Phe Lys Val Pro Leu Gly Thr Lys Phe Tyr Arg Val Lys Ala
      1570 1575 1580
Val Ser Trp Asn Lys Lys Val
1585 1590
```

FIP200, a protein of 1591 amino acids with a calculated molecular mass of 200 kDa, is described in Ueda et al., “Suppression of Pyk2 Kinase and Cellular Activities by FIP200,” Cell Biology 149(2):423-430 (2000); Abbi et al., “Regulation of Focal Adhesion Kinase by a Novel Protein Inhibitor FIP200,” Molecular Biology of the Cell 13:3178-3191 (2002), which are hereby incorporated by reference in their entirety. This protein is encoded by a nucleic acid molecule which has a nucleotide sequence of SEQ ID NO:2 as follows:

atgaagttatatgtatttctggttaacactggaactactctaacatttga
cactgaacttacagtgcaaactgtggcagaccttaagcatgccattcaaa
gcaaatacaagattgctattcaacaccaggtgctggtggtcaatggagga
gaatgcatggctgcagatcgaagagtgtgtacctacagtgctgggacgga
tacaaatccaatttttctttttaacaaagaaatgatcttatgtgatcgtc
cacctgctattcctaaaactaccttttcgacagaaaatgacatggaaata
aaagttgaagaatctcttatgatgcctgcagtttttcatactgttgcttc
aaggacacagcttgcattggaaatgtatgaagttgccaagaaactttgtt
ctttttgtgaaggtcttgtacatgatgaacatcttcaacaccaaggctgg
gctgcaatcatggccaacctggaggactgttcaaattcataccaaaagct
acttttcaagtttgaaagtatttattcaaattatctgcagtccatagaag
acatcaagttaaaacttactcatttaggaactgcagtttcagtaatggcc
aagattccactgttggagtgcctaaccagacatagttacagagaatgttt
gggaagactggattctttacctgaacatgaagactcagaaaaagctgaga
cgaaaagatccactgaactggtgctctctcctgatatgcctagaacaact
aacgaatctttgttaacctcatttcccaagtcagtggaacatgtgtcccc
agataccgcagatgctgaaagtggcaaagaaattagggaatcttgtcaaa
gtactgttcatcagcaagatgaaactacgattgacactaaagatggtgat
ctgcccttttttaatgtctctttgttagactggataaatgttcaagatag
acctaatgatgtggaatctttggtcaggaagtgctttgattctatgagca
ggcttgatccaaggattattcgaccatttatagcagaatgccgtcaaact
attgccaaacttgataatcagaatatgaaagccattaaaggacttgaaga
tcggctctacgccctggaccagatgattgctagctgtggccgactggtga
atgaacagaaagagcttgctcagggatttttagctaatcagaagagagct
gaaaacttaaaggatgcatctgtattacctgatttatgcctgagtcacgc
aaatcagttgatgattatgttgcaaaatcatagaaaactgttagatatta
agcagaagtgtaccactgccaaacaagaactagcaaataacctacatgtc
agactgaagtggtgttgctttgtaatgcttcatgctgatcaagatggaga
gaagttacaagctttgctccgcctcgtaatagagctgttagaaagagtca
aaattgttgaagctcttagtacagttcctcagatgtactgcttagctgtt
gttgaggttgtaagaagaaaaatgttcataaaacactacagggagtgggc
tggtgctttagtcaaagatggaaagagattatatgaagcagaaaaatcaa
aaagggaatcctttgggaaattatttaggaagtcttttttaagaaatcgt
ctgtttaggggactggactcctggcccccttccttttgtactcaaaagcc
tcgaaagtttgactgtgaacttccagatatttcattaaaagatttacagt
ttctgcaatcattttgtccttcggaagttcagccattcctcagggttccc
ttactttgtgactttgaacctctacaccagcatgtacttgctctacataa
tttggtaaaagcagcacaaagtttggatgaaatgtcacagaccattacag
atctactgagtgaacaaaaggcatctgtgagtcagacatccccacagtct
gcttcttcaccaaggatggaaagtacagcaggaattacaactactacctc
accgagaactcctccaccactgactgttcaggatcccttatgtcctgcag
tttgtcccttagaagaattatctccagatagtattgatgcacatacgttt
gattttgaaactattccccatccaaacatagaacagactattcaccaagt
ttctttagacttggattcattagcagaaagtcctgaatcagattttatgt
ctgctgtgaatgagtttgtaatagaagaaaatttgtcgtctcctaatcct
ataagtgatccacaaagcccagaaatgatggtggaatcactttattcatc
agttatcaatgcgatagacagtagacgaatgcaggatacaaatgtatgtg
gtaaggaggattttggagatcatacttctctgaatgtccagttggaaaga
tgtagagttgttgcccaagactctcacttcagtatacaaaccattaagga
agacctttgccactttagaacatttgtacaaaaagaacagtgtgacttct
caaattcattaaaatgtacagcagtagaaataagaaacattattgaaaaa
gtaaaatgttctctggaaataacactaaaagaaaaacatcaaaaagaact
actgtctttaaaaaatgaatatgaaggtaaacttgacggactaataaagg
aaactgaagagaatgaaaacaaaattaaaaaattgaagggagagttagta
tgccttgaggaggttttacaaaataaagataatgaatttgctttggttaa
acatgaaaaagaagctgtaatctgcctgcagaatgaaaaggatcagaagt
tgttagagatggaaaatataatgcactctcaaaattgtgaaattaaagaa
ctgaagcagtcacgagaaatagtgttagaagacttaaaaaagctccatgt
tgaaaatgatgagaagttacagttattgagggcagaacttcagtccttgg
agcaaagtcatctaaaggaattagaggacacacttcaggttaggcacata
caagagtttgagaaggttatgacagaccacagagtttctttggaggaatt
aaaaaaggaaaaccaacaaataattaatcaaatacaagaatctcatgctg
aaattatccaggaaaaagaaaaacagttacaggaattaaaactcaaggtt
tctgatttgtcagacacgagatgcaagttagaggttgaacttgcgttgaa
ggaagcagaaactgatgaaataaaaattttgctggaagaaagcagagccc
agcagaaggagaccttgaaatctcttcttgaacaagagacagaaaatttg
agaacagaaattagtaaactcaaccaaaagattcaggataataatgaaaa
ttatcaggtgggcttagcagagctaagaactttaatgacaattgaaaaag
atcagtgtatttccgagttaattagtagacatgaagaagaatctaatata
cttaaagctgaattaaacaaagtaacatctttgcataaccaagcatttga
aatagaaaaaaacctaaaagaacaaataattgaactgcagagtaaattgg
attcagaattgagtgctcttgaaagacaaaaagatgaaaaaattacccaa
caagaagagaaatacgaagctattatccagaaccttgagaaagacagaca
aaaattggtcagcagccaggagcaagacagagaacagttaattcagaagc
ttaattgtgaaaaagatgaagctattcagactgccctaaaagaatttaaa
ttggagagagaagttgttgagaaagagttattagaaaaagttaaacatct
tgagaatcaaatagcaaaaagtcctgccattgactctaccagaggagatt
cttcaagcttagttgctgaacttcaagaaaagcttcaggaagaaaaagct
aagtttctagaacaacttgaagagcaagaaaaaagaaagaatgaagaaat
gcaaaatgttcgaacatctttgattgcggaacaacagaccaattttaaca
ctgttttaacaagagagaaaatgagaaaagaaaacataataaatgatctt
agtgataagttgaaaagtacaatgcagcaacaagaacgggataaagattt
gatagagtcactttctgaagatcgagctcgtttgcttgaggaaaagaaaa
agcttgaagaagaagtcagtaagttgcgcagtagcagttttgttccttca
ccatatgtagctacagccccagaactttatggagcttgtgcacctgaact
cccaggtgaatcagatagatccgctgtggaaacagcagatgaaggaagag
tggattcagcaatggagacaagcatgatgtctgtacaagaaaatattcat
atgttgtctgaagaaaaacagcggataatgctgttagaacgaacattgca
attgaaagaagaagaaaataaacggttaaatcaaagactgatgtctcaga
gcatgtcttcagtatcttcaaggcattctgaaaagatagctattagagat
tttcaggtgggagatttggtactcatcatcctagacgaacgccatgacaa
ttatgtgttatttactgttagtcctactttatattttctacattcagagt
ctctacctgccctggatctcaaaccagcttcaggtgcatctagaagaccc
tgggtactcggaaaagtaatggaaaaagaatactgtcaagccaaaaaggc
acaaaacagatttaaagttcctttggggacaaagttttacagagtgaaag
ccgtatcatggaataagaaagtataa

The protein or polypeptide identified herein as NH₂-terminal FIP200 (“NT-FIP”) is useful in accordance with the present invention. NT-FIP is a protein fragment spanning amino acid residues 1 to 638 of SEQ ID NO:1, and has an amino acid sequence of SEQ ID NO:3. This protein is encoded by a nucleic acid molecule which has a nucleotide sequence of SEQ ID NO:4 as follows:

atgaagttatatgtatttctggttaacactggaactactctaacatttga
cactgaacttacagtgcaaactgtggcagaccttaagcatgccattcaaa
gcaaatacaagattgctattcaacaccaggtgctggtggtcaatggagga
gaatgcatggctgcagatcgaagagtgtgtacctacagtgctgggacgga
tacaaatccaatttttctttttaacaaagaaatgatcttatgtgatcgtc
cacctgctattcctaaaactaccttttcgacagaaaatgacatggaaata
aaagttgaagaatctcttatgatgcctgcagtttttcatactgttgcttc
aaggacacagcttgcattggaaatgtatgaagttgccaagaaactttgtt
ctttttgtgaaggtcttgtacatgatgaacatcttcaacaccaaggctgg
gctgcaatcatggccaacctggaggactgttcaaattcataccaaaagct
acttttcaagtttgaaagtatttattcaaattatctgcagtccatagaag
acatcaagttaaaacttactcatttaggaactgcagtttcagtaatggcc
aagattccactgttggagtgcctaaccagacatagttacagagaatgttt
gggaagactggattctttacctgaacatgaagactcagaaaaagctgaga
cgaaaagatccactgaactggtgctctctcctgatatgcctagaacaact
aacgaatctttgttaacctcatttcccaagtcagtggaacatgtgtcccc
agataccgcagatgctgaaagtggcaaagaaattagggaatcttgtcaaa
gtactgttcatcagcaagatgaaactacgattgacactaaagatggtgat
ctgcccttttttaatgtctctttgttagactggataaatgttcaagatag
acctaatgatgtggaatctttggtcaggaagtgctttgattctatgagca
ggcttgatccaaggattattcgaccatttatagcagaatgccgtcaaact
attgccaaacttgataatcagaatatgaaagccattaaaggacttgaaga
tcggctctacgccctggaccagatgattgctagctgtggccgactggtga
atgaacagaaagagcttgctcagggatttttagctaatcagaagagagct
gaaaacttaaaggatgcatctgtattacctgatttatgcctgagtcacgc
aaatcagttgatgattatgttgcaaaatcatagaaaactgttagatatta
agcagaagtgtaccactgccaaacaagaactagcaaataacctacatgtc
agactgaagtggtgttgctttgtaatgcttcatgctgatcaagatggaga
gaagttacaagctttgctccgcctcgtaatagagctgttagaaagagtca
aaattgttgaagctcttagtacagttcctcagatgtactgcttagctgtt
gttgaggttgtaagaagaaaaatgttcataaaacactacagggagtgggc
tggtgctttagtcaaagatggaaagagattatatgaagcagaaaaatcaa
aaagggaatcctttgggaaattatttaggaagtcttttttaagaaatcgt
ctgtttaggggactggactcctggcccccttccttttgtactcaaaagcc
tcgaaagtttgactgtgaacttccagatatttcattaaaagatttacagt
ttctgcaatcattttgtccttcggaagttcagccattcctcagggttccc
ttactttgtgactttgaacctctacaccagcatgtacttgctctacataa
tttggtaaaagcagcacaaagtttggatgaaatgtcacagaccattacag
atctactgagtgaa

The protein or polypeptide identified herein as FIP-N1a is also suitable in accordance with the present invention. FIP-N1a is a protein fragment spanning residues 1 to 154 of SEQ ID NO:1, and has an amino acid sequence of SEQ ID NO:5. This protein is encoded by a nucleic acid molecule which has a nucleotide sequence of SEQ ID NO:6 as follows:

atgaagttatatgtatttctggttaacactggaactactctaacatttga
cactgaacttacagtgcaaactgtggcagaccttaagcatgccattcaaa
gcaaatacaagattgctattcaacaccaggtgctggtggtcaatggagga
gaatgcatggctgcagatcgaagagtgtgtacctacagtgctgggacgga
tacaaatccaatttttctttttaacaaagaaatgatcttatgtgatcgtc
cacctgctattcctaaaactaccttttcgacagaaaatgacatggaaata
aaagttgaagaatctcttatgatgcctgcagtttttcatactgttgcttc
aaggacacagcttgcattggaaatgtatgaagttgccaagaaactttgtt
ctttttgtgaaggtcttgtacatgatgaacatcttcaacaccaaggctgg
gctgcaatcatg

Also suitable in accordance with the present invention is the protein or polypeptide identified herein as middle domain FIP200 (“MD-FIP”). MD-FIP is a protein fragment spanning residues 639-1373 of SEQ ID NO:1, and has an amino acid sequence of SEQ ID NO:7. This protein is encoded by a nucleic acid molecule which has a nucleotide sequence of SEQ ID NO:8 as follows:

caaaaggcatctgtgagtcagacatccccacagtctgcttcttcaccaag
gatggaaagtacagcaggaattacaactactacctcaccgagaactcctc
caccactgactgttcaggatcccttatgtcctgcagtttgtcccttagaa
gaattatctccagatagtattgatgcacatacgtttgattttgaaactat
tccccatccaaacatagaacagactattcaccaagtttctttagacttgg
attcattagcagaaagtcctgaatcagattttatgtctgctgtgaatgag
tttgtaatagaagaaaatttgtcgtctcctaatcctataagtgatccaca
aagcccagaaatgatggtggaatcactttattcatcagttatcaatgcga
tagacagtagacgaatgcaggatacaaatgtatgtggtaaggaggatttt
ggagatcatacttctctgaatgtccagttggaaagatgtagagttgttgc
ccaagactctcacttcagtatacaaaccattaaggaagacctttgccact
ttagaacatttgtacaaaaagaacagtgtgacttctcaaattcattaaaa
tgtacagcagtagaaataagaaacattattgaaaaagtaaaatgttctct
ggaaataacactaaaagaaaaacatcaaaaagaactactgtctttaaaaa
atgaatatgaaggtaaacttgacggactaataaaggaaactgaagagaat
gaaaacaaaattaaaaaattgaagggagagttagtatgccttgaggaggt
tttacaaaataaagataatgaatttgctttggttaaacatgaaaaagaag
ctgtaatctgcctgcagaatgaaaaggatcagaagttgttagagatggaa
aatataatgcactctcaaaattgtgaaattaaagaactgaagcagtcacg
agaaatagtgttagaagacttaaaaaagctccatgttgaaaatgatgaga
agttacagttattgagggcagaacttcagtccttggagcaaagtcatcta
aaggaattagaggacacacttcaggttaggcacatacaagagtttgagaa
ggttatgacagaccacagagtttctttggaggaattaaaaaaggaaaacc
aacaaataattaatcaaatacaagaatctcatgctgaaattatccaggaa
aaagaaaaacagttacaggaattaaaactcaaggtttctgatttgtcaga
cacgagatgcaagttagaggttgaacttgcgttgaaggaagcagaaactg
atgaaataaaaattttgctggaagaaagcagagcccagcagaaggagacc
ttgaaatctcttcttgaacaagagacagaaaatttgagaacagaaattag
taaactcaaccaaaagattcaggataataatgaaaattatcaggtgggct
tagcagagctaagaactttaatgacaattgaaaaagatcagtgtatttcc
gagttaattagtagacatgaagaagaatctaatatacttaaagctgaatt
aaacaaagtaacatctttgcataaccaagcatttgaaatagaaaaaaacc
taaaagaacaaataattgaactgcagagtaaattggattcagaattgagt
gctcttgaaagacaaaaagatgaaaaaattacccaacaagaagagaaata
cgaagctattatccagaaccttgagaaagacagacaaaaattggtcagca
gccaggagcaagacagagaacagttaattcagaagcttaattgtgaaaaa
gatgaagctattcagactgccctaaaagaatttaaattggagagagaagt
tgttgagaaagagttattagaaaaagttaaacatcttgagaatcaaatag
caaaaagtcctgccattgactctaccagaggagattcttcaagcttagtt
gctgaacttcaagaaaagcttcaggaagaaaaagctaagtttctagaaca
acttgaagagcaagaaaaaagaaagaatgaagaaatgcaaaatgttcgaa
catctttgattgcggaacaacagaccaattttaacactgttttaacaaga
gagaaaatgagaaaagaaaacataataaatgatcttagtgataagttgaa
aagtacaatgcagcaacaagaacgggataaagatttgatagagtcacttt
ctgaa

Administration, according to all methods of the present application, may be carried out, without limitation, orally, parenterally, subcutaneously, intravenously, intramuscularly, intraperitoneally, by intravesical instillation, by intracavitary, intraocularly, intraarterially, intralesionally, or by application to mucous membrane, such as, that of the nose, throat, and bronchial tubes. Exemplary delivery devices for all methods herein involving administering a protein or polypeptide include, without limitation, liposomes, transdermal patches, implants, syringes, and gene therapy. Liposomes are vesicles comprised of one or more concentrically ordered lipid bilayers which encapsulate an aqueous phase. They are normally not leaky, but can become leaky if a hole or pore occurs in the membrane, if the membrane is dissolved or degrades, or if the membrane temperature is increased to the phase transition temperature. Current methods of drug delivery via liposomes require that the liposome carrier ultimately become permeable and release the encapsulated drug at the target site. This can be accomplished, for example, in a passive manner wherein the liposome bilayer degrades over time through the action of various agents in the body. Every liposome composition will have a characteristic half-life in the circulation or at other sites in the body and, thus, by controlling the half-life of the liposome composition, the rate at which the bilayer degrades can be somewhat regulated.

In contrast to passive drug release, active drug release involves using an agent to induce a permeability change in the liposome vesicle. Liposome membranes can be constructed so that they become destabilized when the environment becomes acidic near the liposome membrane (see, e.g., Proc. Natl. Acad. Sci. USA 84:7851 (1987); Biochemistry 28:908 (1989), which are hereby incorporated by reference in their entirety). When liposomes are endocytosed by a target cell, for example, they can be routed to acidic endosomes which will destabilize the liposome and result in drug release.

Alternatively, the delivery vehicle includes an enzymatically stable conjugate that includes a polymer. Any protein or polypeptide described herein is chemically conjugated to the polymer.

The liposome membrane can be chemically modified such that an enzyme is placed as a coating on the membrane which slowly destabilizes the liposome. Since control of drug release depends on the concentration of enzyme initially placed in the membrane, there is no real effective way to modulate or alter drug release to achieve “on demand” drug delivery. The same problem exists for pH-sensitive liposomes in that as soon as the liposome vesicle comes into contact with a target cell, it will be engulfed and a drop in pH will lead to drug release. This liposome delivery system can also be made to accumulate at a target organ, tissue, or cell via active targeting. In accordance with the present invention. Liposome can be targeted to an organ, cell or tissue of choice by incorporating into the liposome bilayer a molecule which targets receptors for the organ, tissue, or cell of choice.

Other suitable protein delivery systems may be used, including, without limitation, a transdermal patch, and implantable or injectable protein depot compositions, which provide long-term delivery of fusion proteins (U.S. Pat. No. 6,331,311 to Brodbeck et al., which is hereby incorporated by reference in its entirety). Other delivery systems which are known to those of skill in the art can also be employed to achieve the desired delivery of the fusion protein to the desired organ, tissue, or cells in vivo to effect this aspect of the present invention.

Gene therapy may also be used to administer the protein or polypeptide described herein. Gene therapy involves transforming a suitable host cell with an infective transformation vector harboring the nucleic acid encoding the protein or polypeptide. Exemplary infective transformation vectors include, without limitation, an adenovirus vector or a retrovirus vector harboring a nucleic acid encoding a protein or polypeptide described herein. Such vectors, prepared with suitable transcriptional and translational regulatory elements, are capable of expressing the protein or polypeptide in a transformed cell. The introduction of a protein or polypeptide according to this and all aspects of the present invention involving introducing a protein or polypeptide may be carried out by employing a delivery vehicle having the protein or polypeptide. Exemplary delivery vehicles include, without limitation, a fusion protein having a protein or polypeptide of choice and a ligand domain recognized by the cell of choice; a liposome vehicle, in its various forms as described above and known in the art; or an enzymatically stable conjugate having a polymer and protein or polypeptide conjugated to the polymer. Other delivery vehicles known to those in the art are also suitable.

The FIP200 protein or polypeptide of this and all aspects of the present invention, and its fragments, are preferably produced in purified form by conventional techniques. Typically, the protein or polypeptide is secreted into the growth medium of recombinant E. coli. To isolate the protein or polypeptide, the E. coli host cell carrying a recombinant plasmid is propagated, homogenized, and the homogenate is centrifuged to remove bacterial debris. The supernatant is then subjected to sequential ammonium sulfate precipitation. The fraction containing the desired protein or polypeptide is subjected to gel filtration in an appropriately sized dextran or polyacrylamide column to separate the proteins. If necessary, the protein fraction may be further purified by HPLC. Alternative methods may be used as suitable.

Mutations or variants of the polypeptides or proteins described herein are encompassed by the present invention. Variants may be modified by, for example, the deletion or addition of amino acids that have minimal influence on the properties, secondary structure and hydropathic nature of the polypeptide or protein. For example, a polypeptide or protein may be conjugated to a signal (or leader) sequence at the N-terminal end of the protein or polypeptide which co-translationally or post-translationally directs transfer of the protein or polypeptide. The polypeptide or protein may also be conjugated to a linker or other sequence for ease of synthesis, purification, or identification of the polypeptide or protein.

Fragments of the polypeptides or proteins described herein are also encompassed by the present invention. Suitable fragments can be produced by several means. In the first, subclones of the gene encoding the protein or polypeptide are produced by conventional molecular genetic manipulation by subcloning gene fragments. The subclones then are expressed in vitro or in vivo in bacterial cells to yield a smaller protein or peptide.

In another approach, based on knowledge of the primary structure of any protein or polypeptide described herein, fragments of the gene encoding the protein or polypeptide may be synthesized by using the PCR technique together with specific sets of primers chosen to represent particular portions of the protein or polypeptide. These then are cloned into an appropriate vector for increased expression of an accessory peptide or protein.

Chemical synthesis can also be used to make suitable protein fragments. Such a synthesis is carried out using known amino acid sequences for the protein or polypeptide described herein. These fragments can then be separated by conventional procedures (e.g., chromatography, SDS-PAGE) and used in the methods of the present invention.

The present invention also relates to a method of regulating activity of a kinase. This method involves contacting the kinase with a polypeptide or protein having an amino acid sequence of SEQ ID NO:3, SEQ ID NO:5, or SEQ ID NO:7 under conditions effective to regulate the activity of the kinase.

Exemplary kinases according to this aspect of the present invention include, without limitation, focal adhesion kinase (“FAK”) and other cytoplasmic tyrosine kinases.

Suitable proteins or polypeptides according to this aspect of the present invention are NT-FIP, FIP-N1a, or MD-FIP.

Suitable regulation of a kinase according to this aspect of the present invention includes, without limitation, inhibiting the kinase. Regulating a kinase according to this aspect of the present invention is suitable to regulate fundamental cellular functions, including, without limitation, integrin-mediated cell adhesion, cell migration, cell proliferation, cell cycle progression, and cell spreading.

Inhibiting a kinase according to this aspect of the present invention is also suitable to inhibit downstream phosphorylation of cellular proteins, including, without limitation, cell adhesion-dependent paxillin, squalene-hopene cyclase, a protein having a Crk-associated substrate, and growth factor receptor-bound protein 7.

In this and all aspects of the present invention that involve contacting a kinase with a polypeptide or protein, contact may be effected by any means known in the art or which may be developed hereafter.

Yet another aspect of the present invention is an expression vector including transcriptional and translational regulatory nucleotide sequences operably linked to a nucleic acid molecule having a nucleotide sequence of SEQ ID NO:4, SEQ ID NO:6, or SEQ ID NO:8.

Suitable vectors of the present invention include, without limitation, adenoviral vectors and retroviral vectors. A suitable vector according to the present invention may be constructed by means known in the art. This includes, without limitation, inserting a suitable nucleic acid molecule described herein into an expression system to which the nucleic acid molecule is heterologous (i.e., not normally present). The vector contains the necessary elements for the transcription and translation of the inserted protein-coding sequences. In preparing the nucleic acid construct, the nucleic acid molecule may be inserted or substituted into a bacterial plasmid-vector. Any convenient plasmid may be employed, which will be characterized by having a bacterial replication system, a marker which allows for selection in a bacterium and generally one or more unique, conveniently located restriction sites. Numerous plasmids, referred to as transformation vectors, are available for transformation. Suitable vectors include, but are not limited to, the following: viral vectors, such as lambda vector system gt11, gt WES.tB, Charon 4, and plasmid vectors such as pBR322, pBR325, pACYC177, pACYC1084, pUC8, pUC9, pUC18, pUC19, pLG339, pR290, pKC37, pKC101, SV 40, pBluescript II SK +/− or KS +/− (see “Stratagene Cloning Systems” Catalog (1993) from Stratagene, La Jolla, Calif., which is hereby incorporated by reference in its entirety), pQE, pIH821, pGEX, pET series (see F. W. Studier et. al., “Use of T7 RNA Polymerase To Direct Expression of Cloned Genes,” Gene Expression Technology vol. 185 (1990), which is hereby incorporated by reference in its entirety), and any derivatives thereof. The selection of a vector will depend on the preferred transformation technique and target cells for transfection.

Certain “control elements” or “regulatory sequences” are also incorporated into the plasmid-vector constructs of the present invention. These include non-transcribed regions of the vector and 5′ and 3′ untranslated regions, which interact with host cellular proteins to carry out transcription and translation. Such elements may vary in their strength and specificity. Depending on the vector system and host utilized, any number of suitable transcription and/or translation elements, including constitutive, inducible, and repressible promoters, as well as minimal 5′ promoter elements may be used.

A constitutive promoter is a promoter that directs constant expression of a gene in a cell. Examples of some constitutive promoters that are widely used for inducing expression of transgenes include the nopoline synthase (“NOS”) gene promoter, from Agrobacterium tumefaciens (U.S. Pat. No. 5,034,322 to Rogers et al., which is hereby incorporated by reference in its entirety), the cauliflower mosaic virus (“CaMV”) 35S and 19S promoters (U.S. Pat. No. 5,352,605 to Fraley et al., which is hereby incorporated by reference in its entirety), those derived from any of the several actin genes, which are known to be expressed in most cells types (U.S. Pat. No. 6,002,068 to Privalle et al., which is hereby incorporated by reference in its entirety), and the ubiquitin promoter (“ubi”), which is the promoter of a gene product known to accumulate in many cell types. Examples of constitutive promoters for use in mammalian cells include the RSV promoter derived from Rous sarcoma virus, the CMV promoter derived from cytomegalovirus, β-actin and other actin promoters, and the EF1α promoter derived from the cellular elongation factor 1α gene.

Also suitable as a promoter in the plasmids of the present invention is a promoter that allows for external control over the regulation of gene expression. One way to regulate the amount and the timing of gene expression is to use an inducible promoter. Unlike a constitutive promoter, an inducible promoter is not always optimally active. An inducible promoter is capable of directly or indirectly activating transcription of one or more DNA sequences or genes in response to an inducer. Some inducible promoters are activated by physical means such as the heat shock promoter (“Hsp”). Others are activated by a chemical, for example, IPTG or tetracycline (“Tet on” system). Other examples of inducible promoters include the metallothionine promoter, which is activated by heavy metal ions, and hormone-responsive promoters, which are activated by treatment of certain hormones. In the absence of an inducer, the nucleic acid sequences or genes under the control of the inducible promoter will not be transcribed or will only be minimally transcribed. When any plasmids of the present invention contain an inducible promoter, the method of the present invention further includes the step of adding an appropriate inducing agent to the cell culture when activation of the promoter is desired. Promoters of the nucleic acid construct according to the present invention may be either homologous (derived from the same species as the host cell) or heterologous (derived from a different species than the host cell).

A suitable nucleic acid molecule according to this aspect of the present invention, a promoter molecule of choice, a suitable 3′ regulatory region, and if desired, a reporter gene, are incorporated into a vector-expression system according to this aspect of the present invention to prepare the nucleic acid construct using standard cloning procedures known in the art, such as described by Sambrook et al., Molecular Cloning: A Laboratory Manual Third Edition, Cold Spring Harbor: Cold Spring Harbor Laboratory Press, New York (2001), which is hereby incorporated by reference in its entirety, and U.S. Pat. No. 4,237,224 to Cohen and Boyer, which is hereby incorporated by reference in its entirety, which describes the production of expression systems in the form of recombinant plasmids using restriction enzyme cleavage and ligation with DNA ligase. The transcriptional and translational elements are operably linked to a suitable nucleic acid molecule according to this aspect of the present invention or a fragment thereof, meaning that the resulting vector expresses the desired protein when placed in a suitable host cell. These recombinant plasmids are then introduced by means of transformation and replicated in unicellular cultures including prokaryotic organisms and eukaryotic cells grown in tissue culture.

In one aspect of the present invention, a suitable nucleic acid molecule according to this aspect of the present invention is inserted into the expression system or vector in proper sense (i.e., 5′→3′) orientation and correct reading frame.

Once the isolated suitable nucleic acid molecule has been cloned into an expression system, it is ready to be incorporated into a host cell. Such incorporation can be carried out by the various forms of transformation noted above, depending upon the vector/host cell system. Suitable host cells include, but are not limited to, bacteria, virus, yeast, insect, and mammalian cells, including human. Recombinant molecules can be introduced into cells via transformation, particularly transduction, conjugation, mobilization, or electroporation. The nucleic acid sequences are cloned into the host cell using standard cloning procedures known in the art, as described by Sambrook et al., Molecular Cloning: a Laboratory Manual Third Edition, Cold Springs Laboratory, Cold Springs Harbor, N.Y. (2000), which is hereby incorporated by reference in its entirety.

The present invention also relates to a method of regulating G1 to S phase progression of a cell. This method involves contacting the cell with a polypeptide or protein having an amino acid sequence of SEQ ID NO:3, SEQ ID NO:5, or SEQ ID NO:7 under conditions effective to regulate G1 to S phase progression of the cell. Suitable cells according to this aspect of the present invention include, without limitation, a human breast cancer cell or a mammary epithelial cell.

In this and all aspects of the present invention that involve contacting a cell with a polypeptide or protein, contacting may be effected by any means currently known in the art, or developed hereafter.

Another aspect of the present invention is a method of regulating expression of p21 in a cell. This method involves contacting the cell with a polypeptide or protein having an amino acid sequence of SEQ ID NO:3, SEQ ID NO:5, or SEQ ID NO:7 under conditions effective to regulate expression of p21. A suitable cell according to this aspect of the present invention is, without limitation, a human breast cancer cell.

Yet another aspect of the present invention is a method of regulating phosphorylation of retinoblastoma protein in a cell. This method involves contacting the cell with a polypeptide or protein having an amino acid sequence of SEQ ID NO:3, SEQ ID NO:5, or SEQ ID NO:7 under conditions effective to regulate phosphorylation of retinoblastoma protein. A suitable cell according to this aspect of the present invention is, without limitation, a human breast cancer cell.

The present invention also relates to a method of regulating retinoblastoma protein/E2F transcription factor 1 complex formation in a cell. This method involves contacting the cell with a polypeptide or protein having an amino acid sequence of SEQ ID NO:3, SEQ ID NO:5, or SEQ ID NO:7 under conditions effective to regulate retinoblastoma protein/E2F transcription factor 1 complex formation. A suitable cell according to the present invention is, without limitation, a human breast cancer cell.

Another aspect of the present invention is a method of regulating detachment-induced apoptosis of a cell. This method involves contacting the cell with a protein or polypeptide having an amino acid sequence of SEQ ID NO:3, SEQ ID NO:5, or SEQ ID NO:7 under conditions effective to regulate detachment-induced apoptosis of the cell. A suitable cell according to this aspect of the present invention is, without limitation, a human breast cancer cell.

Yet another aspect of the present invention is a method of regulating anchorage-independent growth of a cell. This method involves contacting the cell with a protein or polypeptide having an amino acid sequence of SEQ ID NO:3, SEQ ID NO:5, or SEQ ID NO:7 under conditions effective to regulate anchorage-independent growth of the cell. A suitable cell according to this aspect of the present invention is, without limitation, a human breast cancer cell.

The present invention also relates to a method of regulating tumor formation in a subject. This method involves administering a therapeutically effective amount of a protein or polypeptide having an amino acid sequence of SEQ ID NO:3, SEQ ID NO:5, or SEQ ID NO:7 to the subject under conditions effective to regulate tumor formation in the subject. Subjects suitable for this aspect of the present invention include, but are not limited to, mammals, including humans. A suitable tumor according to this aspect of the present invention is, without limitation, a breast cancer tumor.

As described in the Examples, the polypeptides or proteins described herein are useful for inhibiting the formation of neoplastic cells, and are, therefore, useful for regulating the formation of solid tumors, including, without limitation, sarcomas and carcinomas, such as astrocytomas, prostate cancer, breast cancer, small cell lung cancer, and ovarian cancer, leukemias, lymphomas, adult T-cell leukemia/lymphoma, and other neoplastic disease states.

Another aspect of the present invention is a method of regulating tumor growth in a subject. This method involves administering a therapeutically effective amount of a protein or polypeptide having an amino acid sequence of SEQ ID NO:3, SEQ ID NO:5, or SEQ ID NO:7 to the subject under conditions effective to regulate tumor growth. Subjects suitable for this aspect of the present invention include, but are not limited to, mammals, including humans. A suitable tumor according to this aspect of the present invention is, without limitation, a breast cancer tumor.

As described in the Examples, the polypeptides or proteins described herein are useful for inhibiting the growth of neoplastic cells, and are, therefore, useful for regulating the growth of solid tumors, including, without limitation, those described herein above.

Any of the above methods involving the use of the proteins or polypeptides of the present invention can, instead, be carried out using the encoding nucleic acid molecules. In this case, the above-described gene therapy procedures can be utilized. For instance, yet another aspect of the present invention is a method of regulating tumor formation in a subject. This method involves administering a therapeutically effective amount of a nucleic acid molecule having a nucleotide sequence of SEQ ID NO:4, SEQ ID NO:6, or SEQ ID NO:8 to a subject under conditions effective to regulate tumor formation. Subjects suitable for this aspect of the present invention include, but are not limited to, mammals, including humans. A suitable tumor according to this aspect of the present invention is, without limitation, a breast cancer tumor. Methods of administration suitable for this aspect of the present invention include those described above.

The present invention also relates to a method of regulating tumor growth in a subject. This method involves administering a therapeutically effective amount of a nucleic acid molecule having a nucleotide sequence of SEQ ID NO:4, SEQ ID NO:6, or SEQ ID NO:8 to the subject under conditions effective to regulate tumor growth. Subjects suitable for this aspect of the present invention include, but are not limited to, mammals, including humans. A suitable tumor according to the present invention is, without limitation, a breast cancer tumor. Methods of administration suitable for this aspect of the present invention include those described above.

### EXAMPLES

### Example 1

### Preparation of Antibodies

Polyclonal antibodies against the C-terminal FIP200 (residues 1374-1591; anti-FIP200C; Ueda et al., “Suppressions of Pyk2 Kinase and Cellular Activities by FIP200,” J. Cell. Biol. 149:423-430 (2000), which is hereby incorporated by reference in its entirety), rabbit antiserum against FAK (Chen et al., “Association of Focal Adhesion Kinase With Its Potential Substrate Phosphatidylinositol 3-Kinase,” Proc. Natl. Acad. Sci. USA 91:10148-10152 (1994), which is hereby incorporated by reference in its entirety), mouse mAb KT3 (Cary et al., “Stimulation of Cell Migration by Overexpression of Focal Adhesion Kinase and Its Association With Src and Fyn,” J. Cell Sci. 109:1787-1794 (1996), which is hereby incorporated by reference in its entirety), and mouse mAb 12CA5 that recognize the hemagglutinin (HA) epitope tag (Chen et al., “Interaction of Focal Adhesion Kinase With Cytoskeletal Protein Talin,” J. Biol. Chem. 270:16995-16999 (1995), which is hereby incorporated by reference in its entirety) have been described previously. Antiserum against the N-terminal segment of FIP200 was prepared in rabbits using a glutathione S-transferase (GST)-fusion protein containing residues 1-373 within the N-terminus of FIP200. Anti-FIP200N antibodies were affinity purified from the antiserum using the same fusion protein immobilized on glutathione-Sepharose as an affinity matrix. Antiphosphotyrosine antibody (PY20) and mouse mAbs against FAK, Pyk2, and paxillin, were purchased from Transduction Laboratories (Lexington, Ky.). Rabbit antibody against phosphorylated Y397 of FAK (anti-pFAKY397) was purchased from Biosource (Camarillo, Calif.). Rabbit anti-HA (HA probe), mouse mAb against Myc epitope tag (9E10), and rabbit polyclonal anti-green fluorescent protein 8 were obtained from Santa Cruz Biotechnology (Santa Cruz, Calif.). Rabbit polyclonal anti-β-Gal was from 5 prime-3 prime, Inc. (Boulder, Colo.). Mouse monoclonal anti-Flag, anti-BrdU, fluorescein-conjugated goat anti-rabbit immunoglobulin (Ig) G, and rhodamine-conjugated goat anti-mouse IgG were purchased from Sigma (St. Louis, Mo.).

### Example 2

### Construction of Expression Vectors

The expression vectors pSG5-FIP200, pSG5-N-terminal-FIP 2, and pSG5-C-terminal-FIP (CT-FIP) encoding Flag-tagged full-length NT-FIP and CT-FIP have been described previously (Ueda et al., “Suppressions of Pyk2 Kinase and Cellular Activities by FIP200,” J. Cell. Biol. 149:423-430 (2000), which is hereby incorporated by reference in its entirety). pSG5-middle domain-FIP 4 encoding Flag-tagged middle domain of FIP200 was generated by amplifying residues 639-1373 of FIP200 using primers with EcoRV site at the 5′ end and BglIsite at the 3′ site. The region was subsequently cloned into the corresponding cloning sites in pSG5 vector. Similarly, expression vectors pKH3-FIP200, pKH3-NT-FIP, pKH3-MD-FIP, and pKH3-CT-FIP encoding HA-tagged FIP200 or fragments were generated by amplifying residues 1-1591, 1-638, 639-1373, and 1374-1591 with the addition of SmaI site at 5′ end and EcoRV site at 3′ end. These fragments were subsequently digested and cloned into corresponding cloning sties in pKH3 vector.

pGEX-CT-FIP has been described (Ueda et al., “Suppressions of Pyk2 Kinase and Cellular Activities by FIP200,” J. Cell. Biol. 149:423-430 (2000), which is hereby incorporated by reference in its entirety). pGEX-NT-FIP was constructed by performing polymerase chain reaction (PCR) to generate a 1.1-kb N-terminal fragment corresponding to residues 1-373 within NT-FIP with the addition of SmaI site at the 5′ end and EcoRV site at the 3′ end. This fragment was digested with SmaI and EcoRV and was inserted into the corresponding cloning site of pGEX-2T vector. pGEX-MD-FIP was generated by amplifying region corresponding to residues 639-1373 of plasmid encoding full-length-FIP200. The primers included a SmaI site at 5′ end and EcoRV and was inserted into corresponding sites into the pGEX-2T vector.

FAK segment containing N-terminal domain (NT-FAK) was generated by PCR amplification using the (SEQ ID NO:9) forward (5′-CTGGATCCAT-GGCAGCTTACCTTG-3′) and (SEQ ID NO:10) reverse (5′-ATGATATCTTAAG-TATCTTC TTCATC-3′) primers. The PCR product was digested with BamHI and EcoRV and was cloned into pKH3 at BamHI and SmaI site to generate pKH3-NT-FAK. FAK segment containing the kinase domain (KD-FAK) was generated by PCR amplification using the (SEQ ID NO:11) forward (5′-ATGATATCAACCAGAGATTATGAAATTC-3′) and (SEQ ID NO:12) reverse (5′-GCTTAAATTAAGTAAACCTGGGTCGTC-TAC-3′) primers. The PCR product was digested with EcoRV and DraI and was cloned into pKH3 at SmaI site to generate pKH3-KD-FAK. The same primers were used to amplify the kinase domain with K454 to R mutation using a FAK cDNA with this mutation as the template (Cary et al., “Stimulation of Cell Migration by Overexpression of Focal Adhesion Kinase and Its Association With Src and Fyn,” Cell Sci. 109:1787-1794 (1996), which is hereby incorporated by reference in its entirety). This fragment was then cloned into pKH3 vector to make the HA-tagged KD^{KR} construct. The expression vectors encoding full-length HA-tagged FAK and the C-terminal FAK have been described previously (Zhao et al., “Regulation of the Cell Cycle by Focal Adhesion Kinase,” J. Cell. Biol. 143:1997-2008 (1998), which is hereby incoporporated by reference in its entirety).

The kinase domain of Pyk2 was generated by PCR amplification using the (SEQ ID NO:13) sense (5′-CCAGGATCCGGCATTGCCCGTGAAGATG-3′) and (SEQ ID NO:14) antisense (5′-ATGAATTCGCTTCACACCAGCTCGGTG-3′) oligonucleotides. The product was then inserted into pKH3 to generate pKH3-KD-Pyk2. The vector encoding full-length Pyk2 has been described previously (Zheng et al., “Differential Regulation of Pyk2 and Focal Adhesion Kinase (FAK): The C-Terminal Domain of FAK Confers Response To Cell Adhesion,” J. Biol. Chem. 273:2384-2389 (1998), which is hereby incoporporated by reference in its entirety). The expression vectors encoding HA-taged Grb7 and the control protein (Grb7-SH2 domain) have been described previously (Han et al., “Association of Focal Adhesion Kinase With Grb7 and Its Role in Cell Migration,” J. Biol. Chem. 274:24425-24430 (1999), which is hereby incorporated by reference in its entirety).

### Example 3

### In Vitro Binding

GST fusion proteins were produced and purified using a protease-defective Escherichia coli strain BL21-Dex, as described previously (Ueda et al., “Suppressions of Pyk2 Kinase and Cellular Activities by FIP200,” J. Cell. Biol. 149:423-430 (2000), which is hereby incorporated by reference in its entirety). GST fusion proteins (3 μg) were immobilized on glutathione-agarose beads and were then incubated for 4 hours at 4° C. with lysates (200 μg) prepared from 293 cells that had been transfected with expression vectors encoding kinase domain of Pyk2, HA-FAK, or its fragments. After washing, the bound proteins were analyzed by western blotting with anti-HA (1:2000) as described below. For binding to the recombinant FAK, His-tagged recombinant FAK was purified from baculovirus-infected sf21 cells as described previously (Withers et al., “Expression, Purification and Characterization of Focal Adhesion Kinase Using a Baculovirus System,” Protein Exp. Purif. 7:12-18 (1996), which is hereby incorporated by reference in its entirety). GST-fusion proteins (5 μg) were equalized for amount of glutathione agarose beads and were incubated with 1 μg of purified His-tagged FAK in binding buffer (20 mM Tris, pH 8.0, 137 mM NaCl, 1 mM MgCl₂, and 1% Triton) overnight at 4° C. with rotation. The samples were then washed five times with binding buffer, boiled in SDS buffer, resolved by SDS-PAGE, and western blotted with α-FAK antibody.

### Example 4

### Immunoprecipitation and Western Blot

For most experiments, cells were lysed with 1% NP-40 lysis buffer (20 mM Tris, pH 8.0, 137 mM NaCl, 1% Nonidet P40, 10% glycerol, 1 mM Na₃VO₄, 1 mM phenylmethylsulfoxide (PMSF), 10 μg/ml aprotinin, and 20 μg/ml leupeptin). For experiments to detect phosphorylation of HA-Shc, cells were lysed in the modified RIPA lysis buffer (50 mM Tris, pH 7.5, 150 mM NaCl, 0.3% sodium deoxycholate, 0.1% Nonidet P-40, 10% glycerol, 1.5 mM MgCl₂, 1 mM EDTA, 0.2 mM EGTA, 20 mM NaF, 25 μM ZnCl₂, 1 mM Na₃VO₄, 1 mM PMSF, 10 μg/ml aprotinin, and 2 μg/ml leupeptin) as described previously (Zhao et al., “Regulation of the Cell Cycle by Focal Adhesion Kinase,” J. Cell. Biol. 143:1997-2008 (1998), which is hereby incorporated by reference in its entirety). Immunoprecipitation was carried out at 4° C. by incubating cell lysates for 2-4 hours with indicated antibodies followed by incubation for 1 hour with Protein A-Sepharose or Protein G-Plus. Immunoprecipitates were washed three times in lysis buffer without protease inhibitors. The beads were then resuspended in SDS-PAGE sample buffer, boiled for 5 min, and resolved by SDS-PAGE. Western blotting was performed with appropriate antibodies as indicated, using the Amersham enhanced chemiluminescent system (Arlington Heights, Ill.), as described previously (Chen et al., “Interaction of Focal Adhesion Kinase With Cytoskeletal Protein Talin,” J. Biol. Chem. 270:16995-16999 (1995); Ueda et al., “Suppressions of Pyk2 Kinase and Cellular Activities by FIP200,” J. Cell. Biol. 149:423-430 (2000), which are hereby incorporated by reference in their entirety). In some experiments, whole cell lysates were analyzed directly by western blotting.

### Example 5

### FAK In Vitro Kinase Assay

FAK was immunoprecipitated from Chinese hamster ovary cells overexpressing FAK (Cary et al., “Focal Adhesion Kinase in Integrin-Mediated Signaling,” Front. Biosci. 4:D102-D113 (1999), which is hereby incorporated by reference in its entirety). Aliquots of the immune complex were assayed for kinase activity as described previously (Ueda et al., “Suppressions of Pyk2 Kinase and Cellular Activities by FIP200,” J. Cell. Biol. 149:423-430 (2000), which is hereby incorporated by reference herein) in the presence of various amounts of GST fusion proteins containing FIP200 segments or GST alone.

### Example 6

### Measurement of Cell Spreading

NIH3T3 cells were transfected using the LipofectAmine and PLUS transfection reagents (Life Technologies, Grand Island, N.Y.) according to the manufacturer's instructions. One day after transfection, the cells were replated on FN (10 μg/ml), fixed in formaldehyde, and processed for immunofluorescence staining (see below). Alternatively, cells were cotransfected with a plasmid encoding β-gal activity as described previously (Cary et al., “Stimulation of Cell Migration by Overexpression of Focal Adhesion kinase and Its Association With Src and Fyn,” J. Cell Sci. 109:1787-1794 (1996), which is hereby incorporated by reference in its entirety). At least 60 positively transfected cells (blue) were counted for their spreading phenotype in each transfection in three independent experiments.

### Example 7

### Cell Migration Assays

NIH 3T3 cells were cotransfected with various vectors along with a plasmid encoding GFP in 7:1 ratio using the LipofectAmine and PLUS transfection reagents (Life Technologies) according to the manufacturer's instructions. One or 2 days after transfection, the cell monolayer was wounded with a p10 tip. The plates were then washed and incubated at 37° C. in growth medium for 8 hours. Phase contrast and fluorescence images were taken every 2 hours until the wound closed (˜10 hours). The rate of migration was calculated by measuring the distance moved toward the center of the wound in 8 hours. Motility assays using OMAware were as described previously (Han et al., “Role of Grb7 Targeting To Focal Contacts and Its Phosphorylation by Focal Adhesion Kinase in Regulation of Cell Migration,” J. Biol. Chem. 275:28911-28917 (2000), which is hereby incorporated by reference in its entirety).

### Example 8

### Measurement of Cell Cycle Progression by BrdU Incorporation

BrdU incorporation assays were performed as described previously (Zhao et al., “Regulation of the Cell Cycle by Focal Adhesion Kinase,” J. Cell. Biol. 143:1997-2008 (1998), which is hereby incorporated by reference in its entirety). Briefly, NIH 3T3 cells were transfected using the LipofectAmine and PLUS transfection reagents (Life Technologies) according to the manufacturer's instructions. The subconfluent transfected cells were serum starved for 24 hours in DME with 0.5% CS. They were then replated on FN (10 μg/ml) and incubated for 16 hours with 100 μM BrdU (Sigma) in DME plus 10% CS. For experiments with FAK-KD^{KR} mutant, cells were serum starved for 30 hours in 0.5% serum. They were then replated on FN (10 μg/ml) or poly-L-lysine (PLL; 0.1 mg/ml) and incubated for 20 hours with 100 μM BrdU in 1% serum. Cellular DNA was digested with 0.5 U/μl DNaseI (New England Biolabs, Beverly, Mass.) for 30 min at 37° C. Cells were then processed for double immunofluorescence staining with polyclonal anti-HA (HA probe; 1:300) and monoclonal anti-BrdU (1:300) as described below. At least 80 positively tranfected cells (as recognized by anti-HA) in multiple fields were scored for BrdU staining in each independent experiment. For FAK rescue experiments, an expression plasmid encoding β-Gal was also included in transfections. Cells were then analyzed for BrdU incorporation as described above, except that the positively transfected cells were identified by immunostaining with polyclonal anti-β-Gal. The percentage of BrdU⁺/β-Gal⁺ cells was determined by analyzing 40-50 β-Gal⁺ cells for each transfection in multiple fields.

### Example 9

### Immunofluorescence Staining

Cells were processed for immunofluorescence staining as described previously (Zhao et al., “Regulation of the Cell Cycle by Focal Adhesion Kinase,” J. Cell. Biol. 143:1997-2008 (1998), which is hereby incorporated by reference in its entirety). The primary antibodies used were polyclonal anti-FIP200N (1:200), monoclonal anti-FAK (1:100), polyclonal anti-HA (1:200), polyclonal anti-β-Gal (1:300), monoclonal anti-BrdU (1:200), and monoclonal antivinculin (1:50). The secondary antibodies used were fluorescein-conjugated goat anti-rabbit IgG (1:300) and rhodamine-conjugated goat anti-mouse IgG (1:200). The cells were mounted on Slowfade (Molecular Probes, Eugene, Oreg.) and examined. The image of stained cells was captured using an immunofluorescence microscope (Olympus, Tokyo, Japan) and a charged-coupled device camera.

### Example 10

### Association of Endogenous FIP200 With FAK

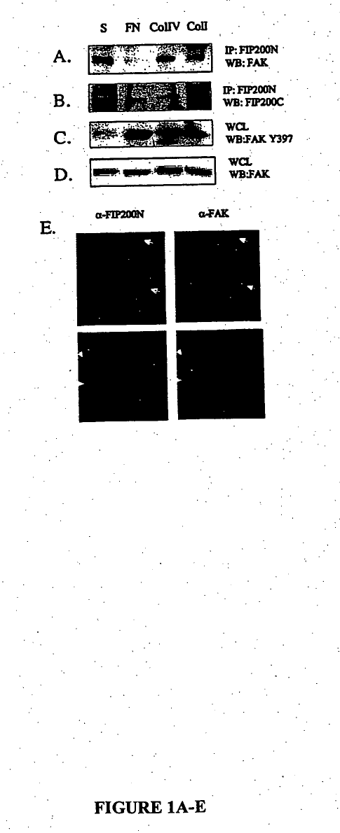

To explore the mechanism and potential function of FIP200 interaction with FAK, interaction of endogenous FIP200 and FAK was analyzed. Lysates were prepared from cells that had been suspended or replated on FN, type IV collagen, or type I collagen. They were immunoprecipitated by an antibody against FIP200 and then subjected to western blotting with anti-FAK to detect associated FAK in the immune complexes. FIG. 1A shows association of endogenous FAK with FIP200 and that the association was decreased upon cell adhesion to FN, and to a lesser extent, type IV collagens or type I collagen. Western blotting of the immunoprecipitates with another antibody against FIP200 showed similar amounts of FIP200 precipitated from cells lysates under these different conditions, as shown in FIG. 1B. Consistent with previous studies (Schwartz et al., “Integrins: Emerging Paradigms of Signal Transduction,” Annu. Rev. Cell. Dev. Biol. 11:549-599 (1995), which is hereby incorporated by reference in its entirety), cell adhesion to FN, and to a lesser extent, type IV collagens or type I collagen, activated FAK that lead to increased FAK autophosphorylation at Y397 (FIGS. 1C, D). These results suggest that FIP200 dissociation from FAK is correlated with FAK activation during cell adhesion, which is consistent with previous finding that FIP200 may also function as a protein inhibitor for FAK (Ueda et al., “Suppressions of Pyk2 Kinase and Cellular Activities by FIP200,” J. Cell. Biol. 149:423-430 (2000), which is hereby incorporated by reference in its entirety). These coimmunoprecipitation analyses also detected association of endogenous FIP200 and FAK in several other cell lines, including rat aortic smooth muscle cells, 293 cells, and NIH 3T3 cells.

Previous studies suggested that FIP200 was predominantly localized in the cytoplasm (Ueda et al., “Suppressions of Pyk2 Kinase and Cellular Activities by FIP200,” J. Cell. Biol. 149:423-430 (2000), which is hereby incorporated by reference in its entirety). Using the new polyclonal antibody against the N-terminal domain of FIP200, presence of endogenous FIP200 was detected in the focal contacts in the cell periphery in addition to the cytoplasmic staining in a fraction of the cells, as shown in FIG. 1E. Costaining with anti-FAK, shown in FIG. 1E (top panels), or anti-vinculin showed partial colocalization of FIP200 with FAK and vinculin in the focal contacts. This partial colocalization of FIP200 with FAK in focal contacts in the periphery of the cells was also seen more clearly in cells transfected with the full-length FIP200, as shown in FIG. 1E (bottom panels). These results suggested that at least part of FIP200 was partially colocalized with FAK.

### Example 11

### FIP200 Association With FAK Through Multiple Interaction Domains

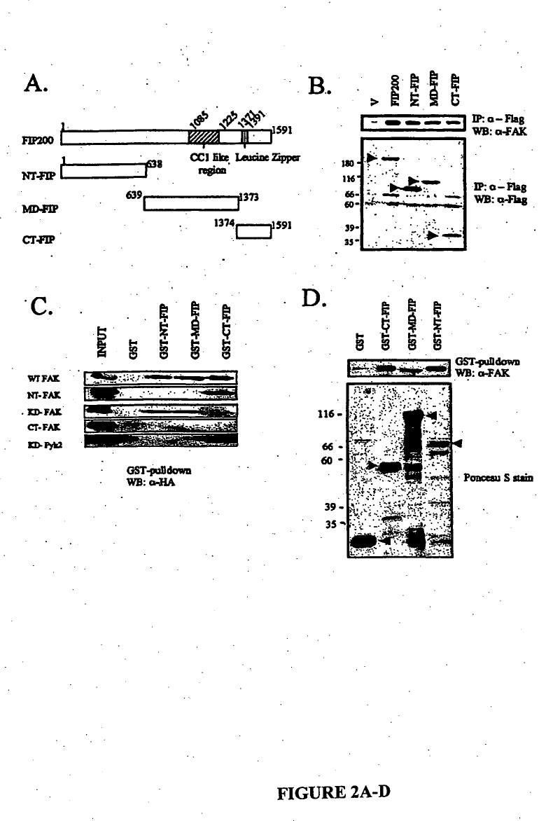

To define the FAK-binding domains within FIP200, HA-tagged FAK was co-expressed with Flag-tagged FIP200 and several FIP200 segments in 293 cells, as shown in FIG. 2A. Immunoprecipitations were performed with anti-Flag antibody and were followed by western blotting with anti-FAK antibody. As shown in FIG. 2B, FAK is coprecipitated with the full-length FIP200 and the CT-FIP, which is consistent with previous results (Ueda et al., “Suppressions of Pyk2 Kinase and Cellular Activities by FIP200,” J. Cell. Biol. 149:423-430 (2000), which is hereby incorporated by reference in its entirety). However, both the FIP200 NT-FIP and MD-FIP segments also associated with FAK in these experiments. In vitro-binding assays were then performed to determine whether all three FIP200 segments bound to the same region on FAK. FIG. 2C shows that GST fusion proteins containing any of the three FIP200 segments bound to the full-length FAK, whereas GST alone did not. GST fusion proteins containing NT-FIP or MD-FIP bound to the kinase domain of FAK, whereas GST fusion protein containing CT-FIP bound to the N-terminal region of FAK. None of the GST fusion proteins bound to the C-terminal region of FAK. The interaction of NT-FIP and MD-FIP with the kinase domain of FAK was specific to FAK because they did not interact with the kinase domain of Pyk2, a homolog of FAK, in the same experiment. Likewise, GST alone did not bind to any of the FAK domains as expected. To examine whether all three FIP200 fragments bound to FAK directly or indirectly through other proteins in the 293 cell lysates, purified recombinant FAK from insect cells was used in the same in vitro-binding assays. FIG. 2D shows that GST fusion proteins containing NT-FIP, MD-FIP, or CT-FIP, but not GST alone, bound to the recombinant FAK. Taken together, these results demonstrate that FIP200 can associate directly and specifically with FAK through multiple interaction domains.

### Example 12

### FIP200 Inhibition of FAK Kinase Activity and Autophosphorylation

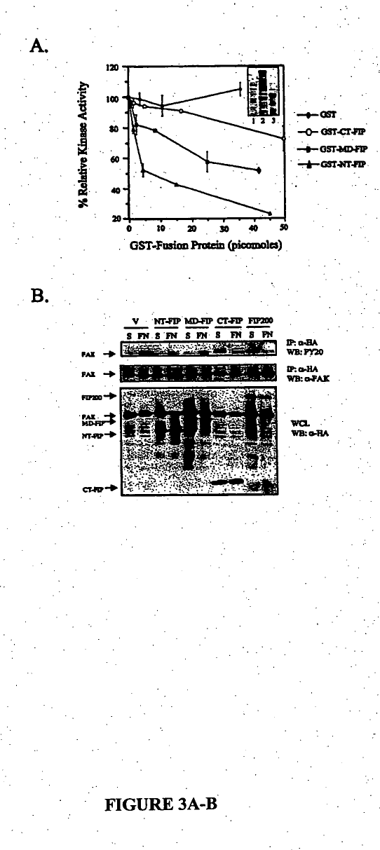

The binding of FIP200 to FAK kinase domain raised the possibility that FIP200 may have an effect on FAK kinase activity. To test this directly, FAK in vitro kinase assays were performed using E4Y1 as an exogenous substrate in the presence of different amounts of purified GST fusion protein containing the FIP200 segments or GST alone as a control. FIG. 3A shows that the GST fusion proteins containing the NT-FIP and MD-FIP inhibited FAK kinase activity, whereas GST alone did not have any effect. GST fusion protein containing NT-FIP showed a significantly greater inhibitory effect than GST fusion protein containing CT-FIP. GST fusion protein containing MD-FIP showed an intermediary activity, which was also insignificantly higher than GST fusion protein containing CT-FIP. In particular, at lower concentrations (e.g., <5 pmol/reaction), GST fusion proteins containing NT-FIP or MD-FIP reduced FAK kinase activity, whereas CT-FIP did not, suggesting that NT-FIP and MD-FIP are more effective than CT-FIP in the inhibition of FAK kinase activity in vitro. These FIP200 segments also inhibited FAK from SYF cells (deficient in Src, Yes, and Fyn expression) to the same extent as FAK from wild-type control cells suggesting that FIP200 inhibited the kinase activity of FAK directly, but not through its potential effects on the associated Src family kinases.

Next examined was the effect of FIP200 and its segments on cell adhesion-induced FAK phosphorylation in intact cells. As shown in FIG. 3B (top and middle panels), expression of FIP200 suppressed tyrosine phosphorylation of FAK after adhesion to FN (compare lanes FIP200 and V). Expression of NT-FIP or MD-FIP also inhibited FAK phosphorylation, as shown in FIG. 3B (compare lanes NT-FIP and MD-FIP with lane V), whereas CT-FIP did not have any effect (compare lanes CT-FIP and V). Similar expression levels of FIP200 fragments were verified by western blotting with anti-HA, as shown in FIG. 3B (lower panel). Together, these results indicate that binding of FIP200 to FAK through interactions at different domains could inhibit FAK kinase activity in vitro. However, they also suggest that the quantitative difference of the in vitro inhibitory activity of FIP200 segments could lead to a differential inhibition of FAK activity in intact cells by NT-FIP and MD-FIP, but not by CT-FIP.

### Example 13

### Effects of FIP200 on FAK Downstream Signaling

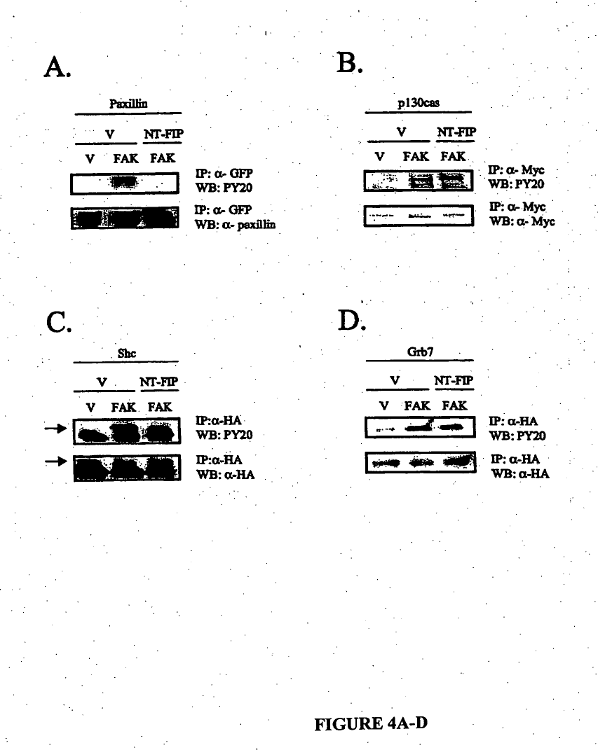

Activation and autophosphorylation of FAK have been suggested to lead to tyrosine phosphorylation of several other cellular proteins, including paxillin, p130cas, Grb7, and Shc (Burridge et al., “Tyrosine Phosphorylation of Paxillin and pp125FAK Accompanies Cell Adhesion To Extracellular Matrix: A Role in Cytoskeletal Assembly,” J. Cell Biol. 119:893-903 (1992); Schaller et al., “pp125FAK-Dependent Tyrosine Phosphorylation of Paxillin Creates a High-Affinity Binding Site for Crk,” Mol. Cell Biol. 15:2635-2645 (1995); Tachibana et al., “Tyrosine Phosphorylation of Crk-Associated Substrates by Focal Adhesion Kinase: A Putative Mechanism for the Integrin-Mediated Tyrosine Phosphorylation of Crk-Associated Substrates,” J. Biol. Chem. 272:29083-29090 (1997); Schlaepfer et al., “Multiple Grb2-Mediated Integrin-Stimulated Signaling Pathways To ERK2/mito-Gen-Activated Protein Kinase: Summation of Both c-Src- and Focal Adhesion Kinase-Initiated Tyrosine Phosphorylation Events,” Mol. Cell. Biol. 18:2571-2585 (1998); Han et al., “Role of Grb7 Targeting To Focal Contacts and its Phosphorylation by Focal Adhesion Kinase in Regulation of Cell Migration,” J. Biol. Chem. 275:28911-28917 (2000), which are hereby incorporated by reference in their entirety). Therefore, the effects of FIP200 on the FAK-promoted activation of these downstream targets was examined. FIG. 4 shows that overexpression of FAK induced tyrosine phosphorylation of all four potential substrates, paxillin, p130cas, Grb7, and Shc, as observed previously (Burridge et al., “Tyrosine Phosphorylation of Paxillin and pp125FAK Accompanies Cell Adhesion to Extracellular Matrix: A Role in Cytoskeletal Assembly,” J. Cell Biol. 119:893-903 (1992); Schaller et. al., “pp125FAK-Dependent Tyrosine Phosphorylation of Paxillin Creates a High-Affinity Binding Site for Crk,” Mol. Cell Biol. 15:2635-2645 (1995); Tachibana et al., “Tyrosine Phosphorylation of Crk-Associated Substrates by Focal Adhesion Kinase: A Putative Mechanism for the Integrin-Mediated Tyrosine Phosphorylation of Crk-Associated Substrates,” J. Biol. Chem. 272:29083-29090 (1997); Schlaepfer et al., “Multiple Grb2-Mediated Integrin-Stimulated Signaling Pathways To ERK2/Mito-Gen-Activated Protein Kinase: Summation of Both c-Src- and Focal Adhesion Kinase-Initiated Tyrosine Phosphorylation Events,” Mol. Cell. Biol. 18:2571-2585 (1998); Han et al., “Role of Grb7 Targeting To Focal Contacts and Its Phosphorylation by Focal Adhesion Kinase in Regulation of Cell Migration,” J. Biol. Chem. 275:28911-28917 (2000), which are hereby incorporated by reference in their entirety). Overexpression of NT-FIP, which had maximum inhibition of FAK activation and phosphorylation among the segments (see FIG. 3), reduced cell adhesion-dependent paxillin and Shc phosphorylation by FAK, as shown in FIGS. 3A-C, but had little effect on p130cas and Grb7 phosphorylation, as shown in FIGS. 3B-D. The mechanism of FIP200's selective inhibition of FAK downstream targets is unknown. It is possible that the threshold activity of FAK required for its phosphorylation of these substrates is different. Thus, inhibition of FAK by FIP200 under these experimental conditions could be sufficient to inhibit paxillin and Shc phosphorylation, but not for p130cas and Grb7 phosphorylation.

### Example 14

### Effects of FIP200 on FAK-Dependent Cell Spreading and Migration

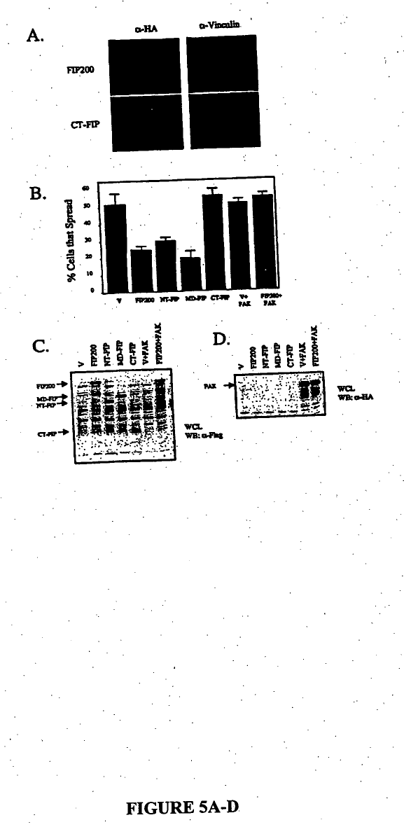

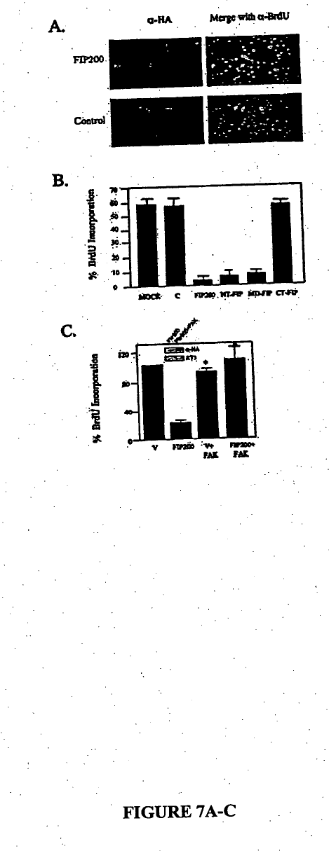

The effects of FIP200 on FAK-regulated cellular functions, including cell spreading and migration, and cell cycle progression, was examined. To study cell spreading, NIH3T3 cells were transiently transfected with the expression vectors encoding FIP200 or its fragments (see FIG. 2A). The effects on cell spreading on FN were assessed initially by immunofluorescent staining with anti-HA antibody to mark the positively transfected cells, and with anti-vinculin antibodies to mark the background untransfected cells in the same field. FIG. 5A shows that transfection of the cells with full-length FIP200 prevented cell spreading on FN (top panels), whereas expression of the CT-FIP did not affect cell spreading (bottom panels). Similar studies showed that expression of NT-FIP or MD-FIP also inhibited cell spreading, although all cells were completely spread after overnight incubation (see FIG. 7A), suggesting that inhibition of cell spreading by FIP200 was transient. Cotransfection of an expression vector encoding β-gal was also used to identify the positively transfected cells in the cell spreading assays. FIG. 5B shows similar results using this method. Expression of FIP-200, NT-FIP, or MD-FIP inhibited cell spreading by ˜50% compared with control untransfected cells or cells expressing CT-FIP. The correlation of cell spreading inhibition by NT-FIP and MD-FIP, but not CT-FIP, with their inhibition of FAK activity (see FIG. 3) suggested that FIP200 might inhibit cell spreading by its inhibition of endogenous FAK functions. Consistent with this possibility, coexpression of FAK with FIP200 rescued inhibition of cell spreading by FIP200 (compare the first and the last lane in FIG. 5B), although overexpression of FAK alone had no effect on cell spreading under these conditions. Western blotting of aliquots of lysates from the transfected cells showed similar expression levels of FIP200 and its fragments and a lack of effects of FAK coexpression on the levels of FIP200, as shown in FIGS. 5C-D.

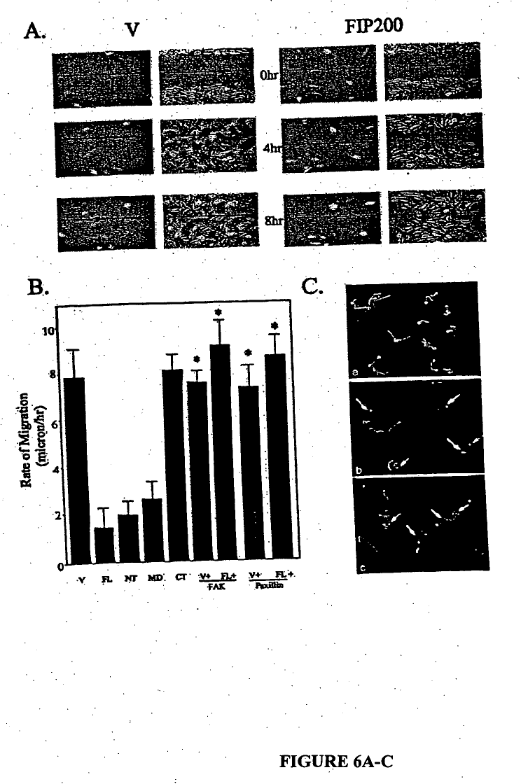

The effect of FIP200 and its fragments on cell migration was assessed by using monolayer-wounding assays after transient transfection of NIH3T3 cells with expression vectors encoding FIP200 or its fragments along with a plasmid encoding GFP. Phase contrast and fluorescence images were captured at regular intervals after wounding to monitor the movement of cells from the wound edge to the center of the wound. The rate of migration was then calculated for transfected cells at the edge of the wound by measuring the distance that the GFP-positive cells moved toward the center of the wound in 8 h. As shown in FIG. 6A, cells transfected with the control vector (V) moved toward the center of the wound at the same rate as the surrounding untransfected cells. In contrast, the FIP200-transfected cells moved much less than the surrounding untransfected cells. Quantification of the rate of migration showed that FIP200, NT-FIP, and MD-FIP inhibited cell migration by ˜60-80%, whereas CT-FIP had no effect, as shown in FIG. 6B. Furthermore, coexpression of FAK or paxillin with FIP200 rescued inhibition of cell migration by FIP200, as shown in FIG. 6B. Similar results were also obtained using an alternative cell migration assays employing a time-lapse imaging-based computerized motility analysis method. OMAware, as described previously (Han et al., “Role of Grb7 Targeting To Focal Contacts and Its Phosphorylation by Focal Adhesion Kinase in Regulation of Cell Migration,” J. Biol. Chem. 275:28911-28917 (2000), which is hereby incorporated by reference in its entirety). Although FIP200 inhibited cell migration, coexpression with FAK reversed this inhibition to control levels, as shown in FIG. 6C. Taken together, these results demonstrate that FIP200 inhibition of FAK leads to inhibition of FAK-dependent cell spreading and migration and suggest that inhibition of paxillin phosphorylation downstream of FAK might be responsible for these effects.

### Example 15

### FIP200 Inhibition of Cell Proliferation and its Rescue by FAK

To explore a potential role for FIP200 in cell cycle progression, NIH3T3 cells were transiently transfected with the expression vectors encoding FIP200 or its fragments (see FIG. 2A), and the extent of BrdU incorporation was measured. FIG. 7A shows that overexpression of FIP200 inhibited cell cycle progression as measured by BrdU incorporation (top panels). Expression of a control vector encoding an irrelevant protein did not affect BrdU incorporation under the same conditions (bottom panels). Quantitative analysis indicated that FIP200 inhibited cell cycle progression by ˜90% compared with cells transfected with the control plasmid or mock-transfected cells, as shown in FIG. 7B. Similar analysis showed that NT-FIP and MD-FIP also inhibited BrdU incorporation to a similar extent as the full-length FIP200, whereas CT-FIP did not have any effect. There was no evidence of apoptosis in any of the transfected cells, suggesting that the cell cycle effects are not due to possible role of FIP200 or its fragments in cell survival or apoptosis.

FAK has been shown to play a role in cell cycle progression (Zhao et al., “Regulation of the Cell Cycle by Focal Adhesion Kinase,” Cell. Biol. 143:1997-2008 (1998), which is hereby incorporated by reference in its entirety), and it has been shown that FIP200 can inhibit FAK activity. Therefore, if overexpression of FAK along with FIP200 could rescue this inhibition of cell cycle progression was examined. FAK alone did not promote cell proliferation under these conditions, but it rescued the inhibition of BrdU incorporation by FIP200 to the control levels, as shown in FIG. 7C. Western blotting of aliquots of cell lysates showed that coexpression of FAK did not affect the expression levels of FIP200, as shown in FIG. 7C (inset). Together, these date indicate that FIP200 inhibition of FAK also leads to inhibition of FAK-dependent cell cycle progression.

### Example 16

### Effects of Disruption of Functional Interation Between Endogenous FIP200 and FAK

The role of FIP200 as a protein inhibitor for FAK was investigated by disrupting the functional interaction of these two proteins. Although FIP200 can associate with FAK through more than one domain (see FIG. 2), FIP200 binding to FAK kinase domain is responsible for its inhibition of FAK kinase and cellular activities in vivo, as shown in FIGS. 3-7. Therefore, a FAK construct, designated KD^{KR}, which contains only the kinase domain of FAK (residues 403-672) with the kinase-defective mutation (K454 to R) was designed. Overexpression of KD^{KR} should titrate out the FIP200 functional binding sites for the FAK kinase domain, thus relieving its inhibition of FAK. The kinase-defective mutant was used instead of the wild-type kinase domain construct to minimize potential effects of expressing this domain (as a kinase) other than its competing with endogenous FAK kinase domain binding to FIP200. This mutation did not affects its binding to FIP200 as KD^{KR} bound to FIP200 as efficiently as its wild-type kinase domain counterpart.

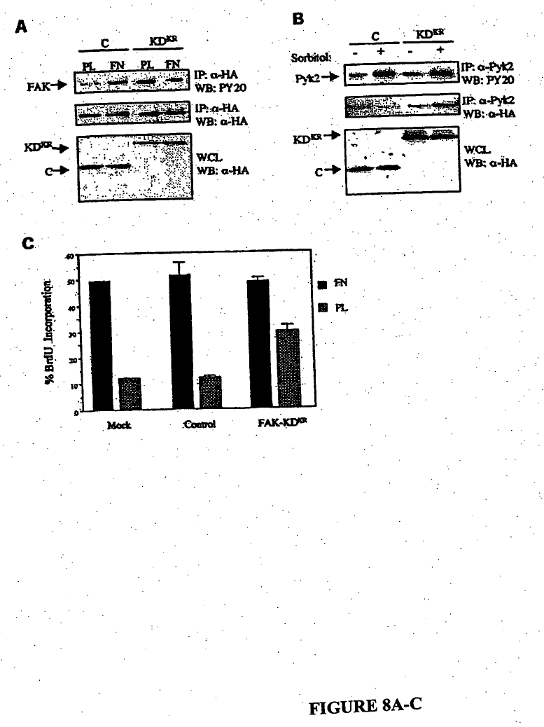

The effects of KD^{KR} on FAK phosphorylation during cell adhesion were examined. Consistent with FIP200 being an inhibitor for FAK, overexpression of KD^{KR} led to an increased tyrosine phosphorylation of FAK in cells plated on PLL in comparison with cells transfected with a control plasmid, as shown in FIG. 8A. The specificity of KD^{KR} to affect FIP200/FAK interaction was supported by its lack of an effect on tyrosine phosphorylation of Pyk2 with or without stimulation by sorbitol, as shown in FIG. 8B. The effects of KD^{KR} on FAK-dependent cell cycle progression was then examined by measuring BrdU incorporation of cells plated on FN or PLL. FIG. 8C shows that a significant fraction (˜50%) of cells plated on FN progressed to the S-phase of cell cycle under the experimental conditions, whereas only a small portion (˜10%) of cells plated on PLL entered the S-phase. In contrast, overexpression of KD^{KR} led to a partial rescue of the reduced cell cycle progression on PLL (˜30%). These results suggest that disruption of FAK inhibition by FIP200 could lead to an increased FAK phosphorylation as well as a partial restoration of cell cycle progression in the absence of cell adhesion to FN. It is likely that additional signals from FN other than FAK phosphorylation are necessary for a full restoration of cell cycle progression. Nevertheless, these data provide further support for FIP200 as a protein inhibitor for FAK.

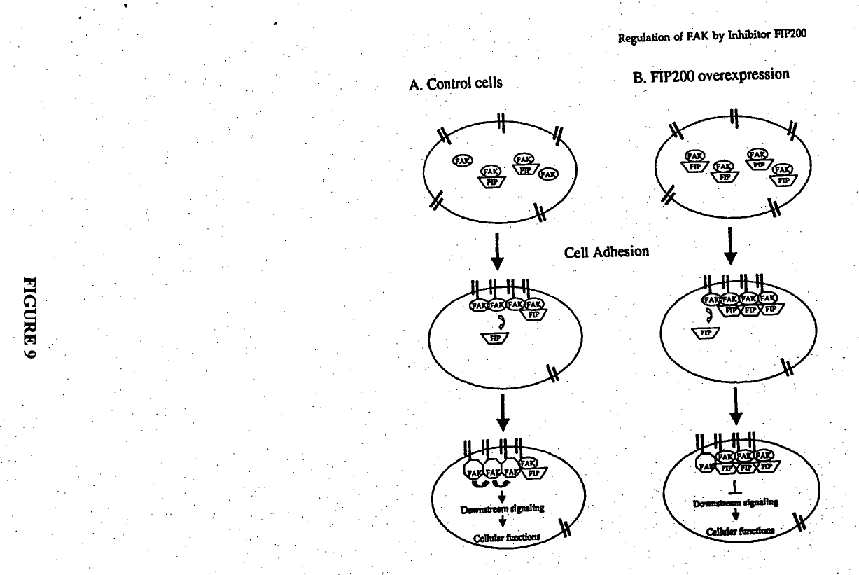

In untransfected control cells, shown in FIG. 9A (left panel), some FIP200 and FAK is complexed under suspended conditions. On cell adhesion and integrin binding to ligands, FIP200 is dissociated from FAK. This release from a negative inhibitor may contribute to FAK activation and phosphorylation in cell adhesion, which trigger downstream signaling pathways in various cellular functions such as cell migration and proliferation. Overexpression of FIP200 in these cells, as shown in FIG. 9B (right panel), drives the equilibrium toward more association of FIP200 with FAK (even in adherent cells), thus leading to inhibition of FAK signaling and function. These hypotheses are consistent with the results shown in FIG. 1, and other observations (see Clark et al., “Integrins and Signal Transduction Pathways: The Road Taken,” Science 268:233-239 (1995); Schwartz et al., “Integrins: Emerging Paradigms of Signal Transduction,” Annu. Rev. Cell Dev. Biol. 11:549-599 (1995); Parsons, J. T., “Integrin-Mediated Signalling: Regulation by Protein Tyrosine Kinases and Small GTP-Binding Proteins,” Curr. Opin. Cell Biol. 8:146-152 (1996); Cary et al., “Focal Adhesion Kinase in Integrin-Mediated Signaling,” Front. Biosci. 4:D102-D113 (1999); Schlaepfer et al., “Signaling Through Focal Adhesion Kinase,” Prog. Biophys. Mol. Biol. 71:435-478 (1999), which are hereby incorporated by reference in their entirety). Although this model implies some role for FIP200 in the regulation of FAK activation by integrins, it is important to note that other factors are also likely to be critical in the activation of FAK by integrins or other receptors.

One potential concern for this proposed role of FIP200 as a protein inhibitor for FAK is that the data are largely based on the overexpression of FIP200 or its fragments. It is possible that proteins of components of positive active complexes might act as dominant inhibitors when overexpressed (e.g., overexpression of the p85 subunit inhibits the PI3K function of the p85/p110 complex). However, the overexpression studies are supported by data from other and complementary approaches. These include the association and regulation of endogenous proteins, as shown in FIG. 1, in vitro studies using purified proteins, shown in FIGS. 2 and 3A, and expression of an FAK segment that disrupts the functional interaction of FIP200 with FAK, as shown in FIG. 8. Given the consistent results from these other approaches, it is very unlikely that the endogenous FIP200 functions as a part of positive FAK complex.

FIP200 inhibited FAK-mediated activation of paxillin and Shc, whereas it had no effect on p130cas and Grb7 phosphorylation in the studies cited herein. It is possible that there is difference in the threshold activity of FAK required to activate its various substrates, and although the inhibition of FAK activity by FIP200 was sufficient to block its activation of paxillin and Shc, it did not effect the activation of other downstream targets. It is also possible that there are separate complexes of FAK with its various substrates, and their interaction with FIP200 is differentially regulated within the cell. In any case, these data suggest that inhibition of FAKmediated tyrosine phosphorylation of paxillin and/or Shc by FIP200 is at least partially responsible for the inhibition of various cellular activities by FIP200. Inhibition of cell spreading by FRNK correlated with a decreased tyrosine phosphorylation of paxillin (Richardson et al., “A Mechanism for Regulation of the Adhesion-Associated Protein Tyrosine Kinase pp125FAK,” Nature 380:538-540 (1996); Richardson et al., “Inhibition of Cell Spreading by Expression of the C-terminal Domain of Focal Adhesion Kinase (FAK) Is Rescued by Coexpression of Src Or Catalytically Inactive FAK: A Role for Paxillin Tyrosine Phosphorylation,” Molecular Biology of the Cell 17:6906-6914 (1997), which are hereby incorporated by reference in their entirety). Furthermore, it has been reported that tyrosine phosphorylation of paxillin and its association with Crk stimulated migration of a tumor cell line NBT-II on collagen (Petit et al., “Phosphorylation of Tyrosine Residues 31 and 118 On Paxillin Regulates Cell Migration Through an Association With CRK in NBT-II Cells,” Cell Biology 148:957-970 (2000), which is hereby incorporated by reference in its entirety). Also, the phosphatase PP2A that dephosphorylates paxillin negatively regulates cell cycle progression and cell motility (Wera et al., “Serine/Threonine Protein Phosphatases,” Biochemistry 311:17-29 (1995); Ito et al., “A Truncated Isoform of the PP2A B56 Subunit Promotes Cell Motility Through Paxillin Phosphorylation,” EMBO 19:562-571 (2000), which are hereby incorporated by reference in their entirety). Consistent with a role for paxillin in cell motility, it has also been observed that overexpression of paxillin rescued FIP200 inhibition of cell migration, as shown in FIG. 6B. Further studies will be necessary to clarify the roles of various FAK-downstream targets in the regulation of cellular activities by FIP200.

Previous studies have shown a number of protein tyrosine phosphatases that inhibit FAK signaling by dephosphorylation of FAK (Arregui et al., “Impaired Integrin-Mediated Adhesion and Signaling in Fibroblasts Expressing a Dominant-negative Mutant PTP1B,” Cell Biology 143:861-873 (1998); Tamura et al., “Inhibition of Cell Migration, Spreading, and Focal Adhesions by Tumor Suppressor PTEN,” Science 280:1614-1617 (1998); Yu et al., “Protein-Tyrosine Phosphatase Shp-2 Regulates Cell Spreading, Migration, and Focal Adhesion,” J. Biol. Chem. 273:21125-21131 (1998); Angers-Loustau et al., “Protein Tyrosine Phospatase-PEST Regulates Focal Adhesion Disassembly, Migration, and Cytokinesis in Fibroblasts,” Cell Biology 144:1019-1031 (1999); Manes et al., “Concerted Activity of Tyrosine Phosphatase SHP-2 and Focal Adhesion Kinase in Regulation of Cell Motility,” Mol. Cell. Biol. 19:3125-3135 (1999); Miao et al., “Activation of EphA2 Kinase Suppresses Integrin Function and Causes Focal-Adhesion-Kinase Dephosphorylation,” Nat. Cell. Biol. 2:62-69 (2000), which are hereby incorporated by reference in their entirety). However, all these inhibitory events required the enzymatic activities of the phosphatases. In contrast, FIP200 inhibits FAK by binding to its kinase domain, which offers the potential opportunity to derive small peptide inhibitors for FAK. Further, two FIP200 segments (NT-FIP and MD-FIP) could both inhibit FAK by apparently similar mechanisms. There are several regions of high homology (˜30% identity) between NT-FIP and MD-FIP. Future studies will be necessary to determine whether these common regions play a role in FIP200 interaction with FAK. The possible generation of small peptides or their derivatives as inhibitors for FAK is another avenue of research, especially because activation of FAK has been implicated in diseases such as cancer metastasis (Weiner et al., “Expression of Focal Adhesion Kinase Gene in Invasive Cancer,” Lancet 342:1024-1025 (1993); Owens et al., “Overexpression of Focal Adhesion Kinase (p125FAK) in Invasive Human Tumors,” Cancer Res. 55:2752-2755 (1995), which are hereby incorporated by reference in their entirety).

### Example 17

### Generation of the TRE2-FIP200 Transgenic Mice

The expression vector (pTRE2-HA-FIP200-IRES2-EGFP-hPG3′) was created using the pTRE2 and pBSK-IRES2-EGFP-hβG3′ plasmids in two steps. First, IRES2-EGFP-hβG3′ was inserted into pTRE2, which received Hemagglutinin-(HA)-tagged FIP200 cDNA in the second step. The doxycycline-regulated expression of this construct was tested in 293T cells.

The TRE2-HA-FIP200-IRES2-EGFP-hβG3′ fragment was injected into fertilized eggs of superovulated FVB mice (The Jackson Laboratory, Bar Harbor, Me.) and pseudopregnant females received these one-cell embryos (Hogan et al., “Production of Transgenic Mice. In Manipulating the Mouse Embryo,” Cold Spring Harbor Laboratory Press, Cold Spring Harbor, N.Y., p. 217 (1994), which is hereby incorporated by reference in its entirety). Thirty-one mice were born. Three transgenic pups were found (2 males and 1 female) after PCR-screening with EGFP-specific primers and Southern blotting using EGFP cDNA.

### Example 18

### Procurement of the MMTV-rtTA Transgenic Mice

Three MMTV-rtTA transgenic mice (two females and one male) (Hsu et al., “Impaired Mammary Gland and Lymphoid Development Caused by Inducible Expression of Axin in Transgenic Mice,” J. Cell Biol. 155:1055 (2001), which is hereby incorporated by reference in its entirety) were obtained through a collaboration with Frank Costantini at (Columbia University, NY, N.Y.).

### Example 19

### Procurement of an MMTV-neu Transgenic Mice Breeding Pair

MMTV-neu (unactivated neu) transgenic mice develop focal mammary tumors at 4 months of age (median incidence: 205 days) in both virgin and breeder mice and 72% percent of tumor-bearing mice that live to 8 months or longer develop metastatic disease to the lung (Muller et al., “Synergistic Interaction of the Neu Proto-Oncogene Product and Transforming Growth Factor Alpha in the Mammary Epithelium of Transgenic Mice,” Mol. Cell Biol. 16:5726 (1996), which is hereby incorporated by reference in its entirety). Cell culture data indicate that FAK and neu signaling pathways interact with each other (Vadlamudi et al., “Differential Regulation of Components of the Focal Adhesion Complex by Heregulin: Role of Phosphatase SHP-2,” Cell Physiol. 190:189 (2002), which is hereby incorporated by reference in its entirety). A breeding pair of MMTV-neu transgenic mice has been purchased from The Jackson Laboratory (Bar Harbor, Me.).

### Example 20

### Testing and Expansion of the TRE2-FIP200 Transgenic Mouse Line

The TRE2-FIP200 founder mice have to transmit the transgene to their offsprings (F1). Moreover, when these F1 mice are mated with the regulator mice (MMTV-rtTA) their offsprings (F2 double transgenic mice, DTg mice) have to have strong, doxycycline-regulated, and mammary gland specific expression of both HA-FIP200 and GFP. The founders and the F1 mice will be selected according to these criteria. The expression of the transgene in other organs will be examined, to compare the expression pattern in these transgenic mice with others (Hsu et al., “Impaired Mammary Gland and Lymphoid Development Caused By Inducible Expression of Axin in Transgenic Mice,” J. Cell Biol. 155:1055 (2001); Leder et al., “Consequences of Widespread Deregulation of the c-myc Gene in Transgenic Mice: Multiple Neoplasms and Normal Development,” Cell 45:485 (1986); Choi et al., “The Mouse Mammary Tumor Virus Long Terminal Repeat Directs Expression in Epithelial and Lymphoid Cells Of Different Tissues in Transgenic Mice,” J. Virol. 61:3013 (1987), which are hereby incorporated by reference in their entirety). In addition to determining the expression pattern, gross and histologic evaluation of the major organ systems and any lesions will be performed.

The TRE2-FIP200 founder mice have been mated with normal FVB mice and PCR screening for the transgene in the F1 mice is being performed. Approximately 50 F1 offsprings of each transgenic founder mice will be screened. The F1 TRE2-FIP200 mice will be mated with the MMTV-rtTA mice to generate F2 DTg offsprings. The F2 mice will be screened with PCR for the presence of both transgenes and then the DTg mice will undergo incisional biopsies of the mammary gland and immunohistochemistry (IHC) and western blotting for HA and GFP will be performed on the biopsy samples. The test group of mice will receive doxyxycline-hydrochloride (Sigma Chemical Co., St. Louis, Mo.) in their drinking water (1 mg/ml concentration with 5% sucrose). The other group will get only drinking water with 5% sucrose. Five days after the doxyxcycline treatment, incisional biopsies of the mammary glands will be taken and again IHC and western blotting for HA and GFP will be done on these biopsy samples. Moreover, western blotting for FIP200 will be performed on paired samples to determine the increase in the level of FIP200 expression. The founders and the F1 mice will be further selected based upon these results. The expression pattern of the transgene will be investigated first with western blotting for HA and GFP and then, in case of a positive result, with IHC and or immunofluorescence (IF). Gross and histologic examination of the DTg mice with or without doxycycline treatment (virgin, pregnant, lactating, and involuting) will typically involve the major organ systems and any gross lesions (if present). Then the founders will be further analyzed to determine copy number, structure, and orientation of the transgene (Nikitin et al., “The Retinoblastoma Gene Regulates Somatic Growth During Mouse Development,” Cancer Res. 61:3110 (2001), which is hereby incorporated by reference in its entirety).

Based upon the previous experience with this system (Hsu et al., “Impaired Mammary Gland and Lymphoid Development Caused by Inducible Expression of Axin in Transgenic Mice,” J. Cell Biol. 155:1055 (2001), which is hereby incorporated by reference in its entirety), it is expected that the expression system will drive the mammary tissue specific expression of HA-FIP200 and GFP in an inducible manner. The HA-expression tag will make it easy to distinguish endogenous and transgenic FIP200 expression. It is expected that the transgene expression in the mammary gland will be adequately high. Because FAK plays a pivotal role in embryonic development (Ilic et al., “Reduced Cell Motility and Enhanced Focal Adhesion Contact Formation in Cells From FAK-Deficient Mice,” Nature 377:539-544 (1995), which is hereby incorporated by reference in its entirety), the transgenic HA-FIP200 expression will only be induced by adding doxycycline-hydrochloride to the drinking water immediately before and during experiments; therefore it is not expected that any gross or histologic abnormality or interference with mammary gland development in the transgenic mice will be encountered, except for certain experiments (see below). According to previous reports, it is expected that the transgene expression will not be limited to the mammary gland (Hsu et al., “Impaired Mammary Gland and Lymphoid Development Caused By Inducible Expression of Axin in Transgenic Mice,” J. Cell Biol. 155:1055 (2001); Leder et al., “Consequences of Widespread Deregulation of the C-Myc Gene in Transgenic Mice: Multiple Neoplasms and Normal Development,” Cell 45:485 (1986); Choi et al., “The Mouse Mammary Tumor Virus Long Terminal Repeat Directs Expression in Epithelial and Lymphoid Cells of Different Tissues in Transgenic Mice,” J. Virol. 61:3013 (1987), which are hereby incorporated by reference in their entirety).

### Example 21

### Effects of the FIP200 Transgene on Lobulo-Alveolar Development of the Mammary Gland During Pregnancy

The lobulo-alveolar development of the mammary gland in late pregnancy is cyclin D1 dependent (Fantl et al., “Mice Lacking Cyclin D1 Are Small and Show Defects in Eye and Mammary Gland Development,” Genes Dev. 9:2364 (1995), which is hereby incorporated by reference in its entirety) and wild-type FAK increases DNA synthesis, accelerates G1/S transition, and increases cyclin D1 expression (Zhao et al., “Regulation of The Cell Cycle by Focal Adhesion Kinase,” J. Cell Biol. 143:1997 (1998), which is hereby incorporated by reference in its entirety). FIP200 inhibits the activities of FAK in vitro (Abbi et al., “Regulation of Focal Adhesion Kinase by a Novel Protein Inhibitor FIP200,” Molecular Biology of the Cell 13:3178-3191 (2001), which is hereby incorporated by reference in its entirety), therefore investigating cyclin D1 expression in DTg mice is a logical step. The morphology of the mammary gland in virgin DTg mice with or without doxycycline treatment will be compared. The maturation process and functioning of the mammary glands in DTg mice during pregnancy and lactation will then be examined.

The morphology of the mammary glands with or without doxycycline treatment will be compared in virgin and pregnant DTg mice. Mammary glands of the pregnant females in both groups will be assessed histologically (sections and whole mounts) at mid pregnancy (day 10-12), at the end of pregnancy (day 18-19), in early lactation (2-day lactation), and in peak lactation (7-day lactation). In addition to histological examination, mRNA will be isolated from females of both groups and quantitative Northern blot analyses for WAP and β-casein mRNA will be performed at these time-points. Also, the newborn pups in both groups will be monitored for presence of milk in their stomach.

It is expected that the expression of HA-FIP200 will have no effect on the morphology of the virgin mouse mammary glands. Due to the requirement for cyclin D1 during lobulo-alveolar development of the mammary gland and due to previous experience that FAK increases cyclin D1 expression and FIP200 inhibits FAK function, it is expected that the DTg females will have hypoplastic mammary glands if the FIP200 transgene is overexpressed. It is expected that the nature of the hypoplasia will be similar to that in mice lacking cyclin D1: the mammary gland will undergo elongation and side branching, but will not form alveolar lobules. As a result of the hypoplasia of the mammary glands, it is expected that the offsprings of DTg mice after doxycycline treatment will have little or no milk in their stomach.

### Example 22

### Effects of the FIP200 Transgene on Mammary Carcinogenesis

Dimethylbenz[a]anthracene (DMBA) will be used to induce mammary gland neoplasia (Pollak et al., “Reduced Mammary Gland Carcinogenesis in Transgenic Mice Expressing a Growth Hormone Antagonist,” Br. J. Cancer 85:428 (2001), which is hereby incorporated by reference in its entirety). It is desirable to know (i) if FIP200 overexpression in the DTg mice reduces FAK phosphorylation; (ii) if FIP200 overexpression in the mammary gland has any effect on the incidence of mammary gland neoplasia after DMBA-treatment; (iii) if FIP200, through its inhibition of FAK can counteract the normal signaling process where FAK-transduced extracellular signals suppress p53-mediated apoptosis (Ilic et al., “Extracellular Matrix Survival Signals Transduced by Focal Adhesion Kinase Suppress p53-Mediated Apoptosis,” J. Cell Biol. 143:547-560 (1998), which is hereby incorporated by reference in its entirety).

The MMTV-FIP200 DTg mice will be crossed with MMTV-neu transgenic mice (Jolicoeur et al., “Use of Mouse Mammary Tumour Virus (MMTV)/Neu Transgenic Mice to Identify Genes Collaborating With the C-erbB-2 Oncogene in Mammary Tumour Development,” Biochem. Soc. Symp. 63:159 (1998), which is hereby incorporated by reference in its entirety) to generate triple-transgenic (TTg) offsprings. This particular transgenic line was chosen, because neu is involved in about 20% of breast carcinomas and in a high proportion of carcinomas in situ. Cell culture data indicate that FAK and neu signaling pathways interact with each other (Vadlamudi et al., “Differential Regulation of Components of the Focal Adhesion Complex by Heregulin: Role of Phosphatase SHP-2,” J. Cell Physiol. 190:189 (2002), which is hereby incorporated by reference in its entirety). Among the many molecular pathological events in MMTV-neu transgenic mice during mammary carcinogenesis is inactivation of tumor suppressor genes. Whether the rate of the loss of heterozygosity (LOH) (Dietrich et al., “Genome-Wide Search for Loss of Heterozygosity in Transgenic Mouse Tumors Reveals Candidate Tumor Suppressor Genes On Chromosomes 9 and 16,” Proc. Natl. Acad. Sci. USA 91:9451 (1994), which is hereby incorporated by reference in its entirety) of p53 changes in these offsprings will be determined.

DMBA (Sigma, St. Louis, Mo.) in cottonseed oil will be given in two 0.5 mg doses administered 7 days apart by gavage and DTg mice in both the test and control groups will be monitored for mammary masses as described (Pollak et al., “Reduced Mammary Gland Carcinogenesis in Transgenic Mice Expressing a Growth Hormone Antagonist,” Br. J. Cancer 85:428 (2001), which is hereby incorporated by reference in its entirety). A veterinary pathologist will perform a complete necropsy and representative tissue samples will be saved both in 10% neutral buffered formalin and at −80° C. The following characteristics will be compared in the test and control groups: (i) The mammary chain and the major organs will be examined both macroscopically and histologically (primary masses and metastases). (ii) IHC for FAK and FIP200 will be performed on the primary tumor (and on the metastatic foci, if present). (iii) Western blotting on tumor samples will be performed to investigate the level of phosphorylation of FAK and related proteins. (iv) TdT-mediated dUTP nick end labeling (TUNEL) assay will be performed to determine if FIP200 has any effect on apoptosis. The results will be interpreted in the light of the level of FAK phosphorylation in the two groups of mice.

The TTg offsprings will be screened for the presence of the three transgenes and for FIP200 transgene expression as described above. The other investigations will be similar to that in the DTg mice after DMBA treatment. Additionally, the detection of LOH will be performed using the microsatellite PCR technique (Dietrich et al., “Genome-Wide Search for Loss of Heterozygosity in Transgenic Mouse Tumors Reveals Candidate Tumor Suppressor Genes On Chromosomes 9 and 16,” Proc. Natl. Acad. Sci. USA 91:9451 (1994), which is hereby incorporated by reference in its entirety).

It is expected that the FIP200 transgene will inhibit FAK phosphorylation in the mammary gland and in the neoplastic mammary masses after doxycycline treatment. It is also expected that the FIP200 transgene induction will result in reduced incidence of mammary neoplasia after DMBA treatment and the neoplastic process will exhibit reduced metastatic spread. It is also expected that mammary tumors in the DTg mice will have reduced apoptotic rate through the inhibitory effect of FIP200 on FAK-induced suppression of p53 mediated apoptosis. It is believed that the TTg mice will have reduced incidence of mammary tumors and reduced metastatic spread of the primary tumor if the FIP200 transgene is overexpressed. It is also expected that the rate of LOH of p53 will be lower in these TTG mice after FIP200 overexpression.

### Example 23

### FIP200 Inhibits G1 to S Phase Transition

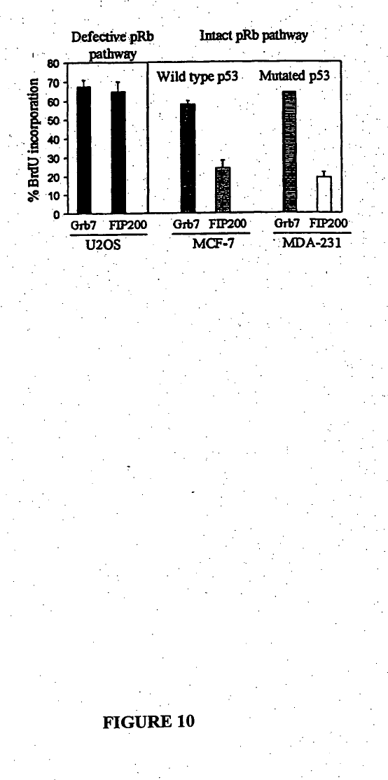

Preliminary results demonstrated that exogenously expressed FIP200 inhibits G1 to S phase progression as measured by BrdU incorporation assay in MCF-7 and MDA-MB-231 human breast cancer cells, as shown in FIG. 10. Because MDA-231 cells have a mutated version of p53 gene it was concluded that FIP200-induced G1 arrest is independent on p53 status. On the other hand, FIP200 was unable to inhibit G1 to S phase transition in U2 OS osterosarcoma cells with the defective pRb pathway, suggesting that intact pRb pathway is required for the FIP200-induced cell cycle arrest, as shown in FIG. 10. Whether FIP200 also affects cell cycle progression in non-tumorigenic mammary epithelial cells, MCF10A, will also be tested. This could not be tested due to the very low transfection efficiency in MCF10A cells with liposomal reagents. This problem should be overcome by using the adenoviral expression system.

### Example 24

### Identification of the Functionally Active Region of FIP200

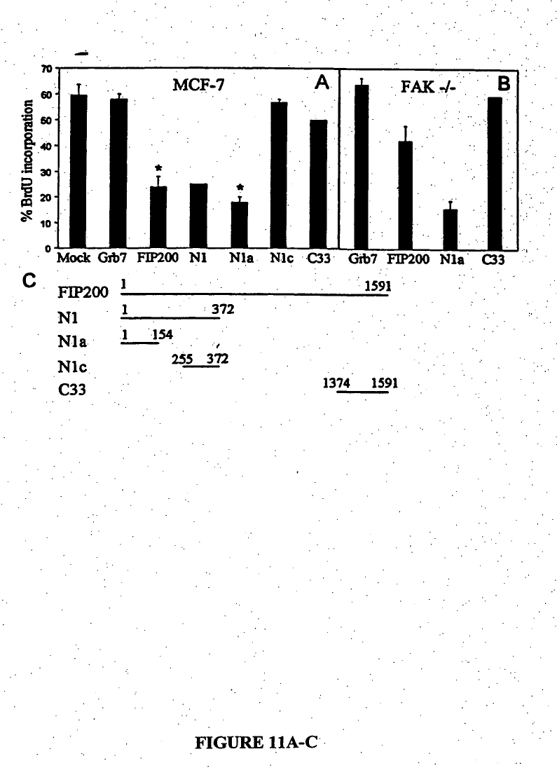

To identify the region of FIP200 that is responsible for G1 arrest, several FIP200 deletion mutants were constructed and their effects on BrdU incorporation in MCF-7 cells tested, as shown in FIGS. 11A, C. A 160 aa N-terminal region of FIP200, N1a, was able to inhibit G1 to S phase transition to the same extent as a full length FIP200, while two other fragments of similar size (N1c and C33) had no effect. In this study it is proposed to further characterize the role of full length FIP200 and N1a in breast tumorigenesis.

### Example 25

### Effect of FIP200 on Different Cell Cycle Regulatory Proteins

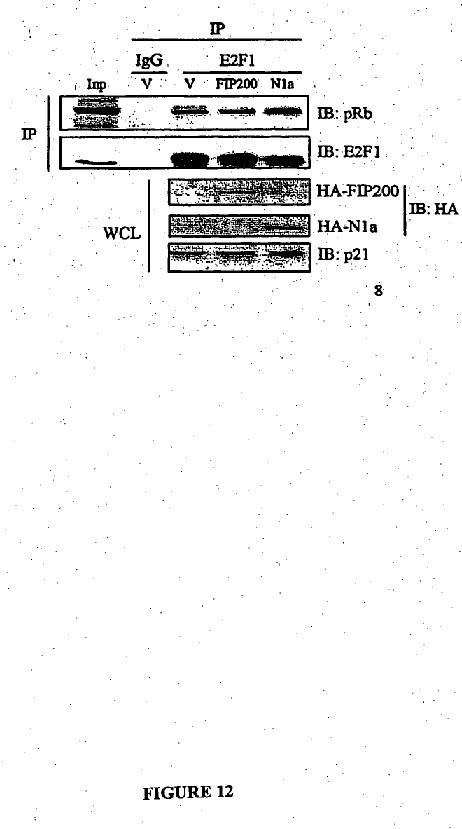

In an attempt to elucidate the molecular mechanisms of FIP200 induced cell cycle arrest, the expression levels of several cell cycle regulatory proteins in response to exogenous expression of FIP200 or its N1a mutant were studied. No changes in protein levels of pRb, E2F1, Cyclin D1, Cyclin E, and p16 in response to either FIP200 or N1a in MCF-7 and MDA-231 cells by western blot analysis were detected. An increase in p21 protein levels by N1a in MDA-231 cells was found, which was accompanied by increased E2F1/pRb complex formation, as shown in FIG. 12. The same changes with full length FIP200 were not detected, perhaps due to the lower expression levels of FIP200 compared to its N1a mutant, probably because of the larger size of FIP200 (1500 aa for FIP200 versus 160 aa for N1a). This problem should be overcome by using the adenoviral gene expression system.

### Example 26

### Role of FAK in FIP200-Induced G1 Arrest

FAK is a cytoplasmic non-receptor tyrosine kinase, which has been shown to play an important role in cell attachment, spreading, migration, cell cycle progression, and detachment-induced apoptosis (Cary et al., “Focal Adhesion Kinase in Integrin-Mediated Signaling,” Frontiers in Bioscience 4:D102-D113 (1999), which is hereby incorporated by reference in its entirety). The role of FAK in breast cancer was suggested by numerous studies, which demonstrated over-expression of FAK in invasive breast tumor specimens compared to the normal tissue form the same patient (Weiner et al., “Expression of Focal Adhesion Kinase Gene in Invasive Cancer,” Lancet 342:1024-1025 (1993); Owens et al., “Overexpression of Focal Adhesion Kinase (p125FAK) in Invasive Human Tumors,” Cancer Res. 55:2752-2755 (1995); Cance et al., “Protein Kinase in Human Breast Cancer,” Breast Cancer Res. Treat. 35:105-114 (1995), which are hereby incorporated by reference in their entirety). Earlier studies demonstrated that FIP200 directly binds to FAK and inhibits its kinase activity. Therefore, it was of interest to test if FAK is required in FIP200-induced G1 arrest. For this purpose, FAK null human embryo fibroblasts were transiently transfected with FIP200, N1a or C33 and their effects on BrdU incorporation were assayed, as shown in FIG. 11B. Full length FIP200 inhibited BrdU incorporation, although to the lesser extent compared to breast cancer cell lines that have wild type FAK. N1a inhibited S phase progression to the same extent in both FAK plus (MCF-7) and FAK minus (embryo fibroblasts) cells. To further clarify the role of FAK in FIP200-induced cell cycle arrest, it is proposed to use the FAK −/− embryo fibroblasts with re-expression of FAK under an inducible promoter. The advantage of this system is that the role of FAK in FIP200-induced cell cycle arrest in the cells with the same genetic background can be tested.

In summary, the background and preliminary data presented above, strongly suggest that FIP200 inhibits G1 to S phase progression in human breast cancer cells and may be involved in the tumorigenesis of breast cancer. This study is proposed to understand the molecular mechanisms of FIP200-induced cell cycle arrest and its role in the development of breast cancer.

### Example 27

### FIP200 is a Putative Tumor Suppressor Gene that Plays an Important Role in Tumorigenesis of Breast Cancer

FIP200 causes cell cycle arrest in human breast cancer cells. FIP200 gene localized in 8q11 chromosome (Chano et al., “Isolation, Characterization and Mapping of the Mouse and Human RB1CC1 Genes,” Gene 291:29-34 (2002), which is hereby incorporated by reference in its entirety), containing several loci of putative tumor suppressor genes, and loss of heterozygosity for this region has been associated with breast cancer (Dahiya et al., “Multiple Sites of Loss of Heterozygosity On Chromosome 8 in Human Breast Cancer Has Differential Correlation With Clinical Parameters,” Int. J. Oncology 12:811-816 (1998), which is hereby incorporated by reference in its entirety). According to one study, 20% (7 of 35) of primary breast cancers contained deletion mutations in FIP200 (Chano et al., “Truncating Mutations of RB1CC1 in Human Breast Cancer,” Nature Genetics 31:285-288 (2002), which is hereby incorporated by reference in its entirety). FIP200 expression closely correlates with pRb tumor suppressor gene expression in various cell lines and tissues (Chano et al., “Identification of RB1CC1, a Novel Human Gene That Can Induce RB1 in Various Human Cells,” Oncogene 21:1295-1298, (2002) which is hereby incorporated by reference in its entirety).

The goal of this study is to understand the mechanisms of cell cycle arrest by FIP200 and its role in tumorigenesis of breast cancer.

### Example 28

### Construct Adenoviral Expression Vectors for FIP200 and its Deletion Mutants and Optimize Cell Infection Conditions

Recombinant adenoviruses expressing full length HA epitope-tagged FIP200 (Ad-FIP200) and its N-terminal (Ad-N1a) or C-terminal (Ad-C33) fragments will be constructed using a commercially available AdEasy system (Stratagene, Inc). Adenovirus expressing GFP, Ad-GFP, will be used as a control. Infection conditions will be optimized to achieve the maximal number of cells expressing the gene of interest with the lowest toxicity. In preliminary experiments with Ad-GFP almost 100% of cells (MDA-231 and MCF-7) expressed GFP compared to about 5-10% achieved with the Lipofectamine Plus transfection reagent.

### Example 29

### Effects Of FIP200 and its Deletion Mutants on Cell Cycle Phase Distribution in a Panel of Breast Cancer Cell Lines and Normal Mammary Epithelial Cells Using the Flow Cytometry Method

Preliminary data showed inhibition of G1 to S phase transition in MCF-7 and MDA-231 cells by FIP200 and N1a using BrdU incorporation assay. To test if the G1 arrest by FIP200 is a general response of different human breast cancer cells and if FIP200 also inhibits G1 to S transition in non-tumorigenic mammary epithelial cells, a panel of human breast cancer cells (MCF-7, MDA-231, MDA-468 pRb −/−, MDA-468 pRb +/+, T47D) and mammary epithelial cell line, MCF10A, will be screened by flow cytometry. Flow cytometery method is preferred to the BrdU incorporation assay because it allows detection of changes in all four phases of the cell cycle as opposed to only G1 to S transition by the BrdU incorporation method. Flow cytometry could not be used in preliminary experiments due to the very low cell transfection efficiency in breast cancer cell lines. This problem will be overcome by using adenoviral expression vectors. The sub-confluent cultures of cells will be infected with Ad-GFP (control), Ad-FIP200, -N1a, or -C33. Twenty-four hours later, cells will be re-plated in 10% FBS to stimulate cell cycle progression and harvested for flow cytometry at 12, 24, and 48 hours after re-plating.

Testing the effects of FIP200 on cell cycle progression in MDA-468 pRb null (MDA-468 pRb −/−) and pRb reconstituted (MDA-468 pRb +/+) (Lu et al., “Evidence For Retinoblastoma Protein (RB) Dependent and Independent IFN-Gamma Responses: RB Coordinately Rescues IFN-Gamma Induction of MHC Class II Gene Transcription in Noninducible Breast Carcinoma Cells,” Oncogene 9(4):1015-1019 (1994), which is hereby incorporated by reference in its entirety), cell lines is critical to confirm preliminary results suggesting that intact pRb pathway is required for FIP200-induced cell cycle arrest. The advantage of this system is that the role of pRb will be tested in the cell lines with the same genetic background.

Similarly, to further investigate if FAK is involved in FIP200-induced cell cycle arrest (as suggested by preliminary data) human embryo fibroblasts with inducible expression of FAK will be used (Zhao et al., “Regulation of the Cell Cycle by Focal Adhesion Kinase,” J. Cell Biol. 143:1997-2008 (1998), which is hereby incorporated by reference in its entirety). The effects of FIP200, N1a, and C33 on cell cycle phase distribution under FAK-induced or un-induced conditions will be tested in a similar experimental design as described for this Example.

It is expected that all human breast cancer cell lines tested will be arrested in G1 phase of the cell cycle by FIP200 and N1a but not GFP or C33. MDA-468 pRb +/+, but not MDA-468 pRb −/− cells will be arrested in G1 phase by FIP200 and N1a, confirming that intact pRb pathway is required for FIP200-induced cell cycle arrest in human breast cancer cells. The inhibition of the cell cycle by FIP200 may have both FAK-dependent and -independent components.

### Example 30

### Effects of FIP200 and its Deletion Mutants on p21 Protein, mRNA Levels and Promoter Activity, as Well as pRb Phosphorylation and pRb/E2F1 Complex Formation

Preliminary data suggest that the cell cycle arrest by FIP200 may be mediated via modulation of the levels of p21, cyclin dependent kinase inhibitor, leading to the increased pRb/E2F1 complex formation. To confirm the functional relevance of p21 in the G1 arrest by FIP200 the ability of FIP200 to induce G1 arrest in p21+/+but not p21 −/− fibroblasts (Zhao et al., “Transcriptional Activation of Cyclin D1 Promoter by FAK Contributes To Cell Cycle Progression,” Mol. Bio. Cell 12:4066-4077 (2001), which is hereby incorporated by reference in its entirety) will be assayed. To understand the molecular mechanisms of p21 regulation by FIP200 and its deletion mutants their effects on p21 expression at the levels of protein, mRNA, and promoter activity will be tested. Sub-confluent cultures of breast cancer (MCF-7, MDA-231) and non-tumorigenic mammary epithelial cells (MCF10A) will be infected with Ad-GFP (control), -FIP200 or its mutants. Twenty-four hours later cells will be re-plated in the 10% FBS and cell extracts will be harvested at 2, 4, 6, 12, 24, and 48 hours for northern and western blot analysis of p21 mRNA and protein levels, respectively. For reporter gene assay, in addition to FIP200 constructs cells will also be transfected with human p21 promoter-luciferase reporter gene construct and cell lysates will be harvested at 12, 24, and 48 hours for the reporter gene assay to test p21 promoter activity. If it is found that p21 promoter activity is increased by FIP200 or N1a, p21 promoter deletion mutants will be constructed to identify the minimal region of p21 promoter (with specific responsive elements) that is sufficient to confer FIP200 responsiveness.

Since one of the roles of p21 is to prevent pRb phosphorylation by CDK4/6 and CDK2 leading to pRb inactivation and dissociation from E2F (Nevins, JR, “The Rb/E2F Pathway and Cancer,” Human Molecular Genetics 10:699-703 (2001), which is hereby incorporated by reference in its entirety), whether an increase in p21 by FIP200 will inhibit pRb phosphorylation (using phospho-pRb specific Ab) and increase pRb/E2F1 complex formation will also be tested (using immunoprecipitation assay, similar to the one described in FIG. 12.

With sufficient expression levels of exogenous proteins by adenoviral vectors it is expected to find an increase in p21 protein levels, pRb phosphorylation and pRb/E2F1 complex formation by both FIP200 and N1a, but not by GFP or C33.

Alternatively, if no effects of FIP200 or N1a on p21 protein levels are found, the role of FAK in FIP200-induced cell cycle arrest will be focused on.

### Example 31

### The Effect of Inducible Expression of the FIP200 and its Mutants on Tumorigenic Properties of Human Breast Cancer Cells In Vitro and In Vivo

To construct MCF-7 and MDA-231 cells with inducible expression of HA-tagged FIP200, N1a, or C33 a commercially available pRevTet System (Clonetech) will be employed. Retroviral pRevTRE vector, PT67 packaging cells, MCF-7 and MDA-231 Tet-off cell lines are available in the lab. For each construct, the single colonies will be selected, expended and expression of the gene of interest under the inducible conditions will be confirmed by western blot analysis using anti-HA Abs.

MCF-7 and MDA-231 cell growth rates under FIP200, N1a or C33 +/− conditions will be assayed by the standard daily cell counting procedure (for up to 10 days) using trypan blue exclusion to discriminate between dead and live cells.

Anchorage-independent growth is one of the main characteristics of the cancer cells, which is required for their detachment from the primary tumor, survival in the bloodstream and formation of the secondary tumors (metastasis). To test the effect of FIP200, N1a or C33 on survival of MCF-7 and MDA-231 cells under anchorage-independent conditions, a soft agar colony formation assay under gene induced or un-induced conditions will be used.

Similarly, the in vitro invasive potential of breast cancer cells under FIP200, N1a or C33 +/− conditions will be assayed by Matrigel invasion assay using 6.5 mm Transwell chambers with 8 um pore size (Zajchowski et al., “Identification of Gene Expression Profiles That Predicts the Aggressive Behavior of Breast Cancer Cells,” Cancer Res. 61:5168-5178 (2001); Zrihan-Licht et al., “RAFT/Pyk2 Tyrosine Kinase Mediates the Association of p190 RhoGAP With RasGAP and Is Involved in Breast Cancer Cell Invasion,” Oncogene 19:1318-1328 (2000), which are hereby incorporated by reference in their entirety). Conditioned NIH3T3 medium will be used as a chemoattractant. After 18 hours, cells that invaded the Matrigel and spread onto the lower surface of the filter will be fixed, stained and counted.

Induction of apoptosis by GFP, FIP200 and its mutants in MCF-7 and MDA-231 cells will be tested by infecting cells with corresponding adenoviruses and screening for apoptosis by the standard Annexin V and Hoechst staining procedures. No technical difficulties are anticipated.

It is expected that in MCF-7 and MDA-231 cells inducible expression of FIP200 and N1a, but not C33 will inhibit or retard cell growth in tissue culture dishes and soft agar and Matrigel invasion.

Nude mice are widely used as an in vivo model to study tumorigenic properties of human breast cancer cells (Price et al., “Studies of Human Breast Cancer Metastasis Using Nude Mice,” Cancer and Metastasis Rev. 8:285-297 (1990), which is hereby incorporated by reference in its entirety). To test if FIP200 and its mutant can suppress mammary tumor growth three week old female athymic nude mice (nu/nu) will be used. Mice will be maintained under pathogen-limiting conditions as described (Gutman et al., “Effects of the Antiestrogen EM-800 (SCH 57050) and Cyclophosphamide Alone and in Combination On Growth of Human ZR-75-1 Breast Cancer Xenografts in Nude Mice,” Cancer Research 59:5176-5180 (1999), which is hereby incorporated by reference in its entirety). MCF-7 and MDA-231 cells expressing FIP200, N1a or C33 will be grown under induced and un-induced conditions for two days, harvested at 80% confluency and 1×10⁶ cells will be injected into mammary fatpad (m.f.p.) of anesthetized mice as described (Bagheri-Yarmand et al., “Carboxymethyl Benzylamide Dextran Blocks Angiogenesis of MDA-MB435 Breast Carcinoma Xenografted in Fat Pad and Its Lung Metastases in Nude Mice,” Cancer Research 59:507-510 (1999), which is hereby incorporated by reference in its entirety). The mice will be fed with +/−0.5 mg/ml doxycycline (induced or un-induced conditions) in the drinking water. Twenty-five to thirty mice will be used per experimental group. The growth of mammary fatpad tumors will be monitored by weekly examination, and tumor size will be determined from caliper measurements of two diameters. After 20-24 weeks, mice will be sacrificed, and isolated tumors will be examined immunohistologically with anti-HA Ab, to confirm the expression of the exogenous gene.

It is expected that FIP200 and N1a, but not C33 will either inhibit of significantly slow down tumor formation and growth in nude mice.

### Example 32

### The Effect of FIP200 Suppression by siRNA Method on Growth Characteristics of Non-Tumorigenic Mammary Epithelial Cell Line, MCF10A

siRNA interference assay is a relatively new technique, which has been successfully used to suppress genes of interest (Sharp, PA, “RNA Interference—2001,” Genes and Development 15(5):485-490 (2001), which is hereby incorporated by reference in its entirety). siRNA oligos targeting different regions of the FIP200 gene will be synthesized using a Silencer siRNA construction kit (Ambion, Inc) according to manufacturer's instructions. siRNA oligos which significantly suppress FIP200 protein levels will be used as described below.

MCF10A is a non-tumorigenic human mammary epithelial cell line, which lacks anchorage-independent growth and Matrigel invasion. To test the effect of FIP200 siRNA on MCF10A growth characteristics the identical experimental approaches as described in Example 31 ((a) cell growth rate in culture dishes, (b) Anchorage-independent growth in soft agar, (c) Matrigel invasion assay) will be employed.

It is expected that if FIP200 is a tumor suppressor, its silencing may render MCF10A cells more tumorigenic by affecting one of the characteristics described above.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

1. A method of treating a subject suffering from a disorder mediated by cell proliferation, said method comprising:
administering a therapeutically effective amount of a polypeptide or protein having an amino acid sequence comprising SEQ ID NO:3, SEQ ID NO:5, or SEQ ID NO:7 to the subject under conditions effective to treat the cell proliferation disorder.

2. The method according to claim 1, wherein the subject is human.

3. The method according to claim 1, wherein the polypeptide or protein comprises an amino acid sequence of SEQ ID NO:3.

4. The method according to claim 1, wherein the polypeptide or protein comprises an amino acid sequence of SEQ ID NO:5.

5. The method according to claim 1, wherein the polypeptide or protein comprises an amino acid sequence of SEQ ID NO:7.

6. The method according to claim 1, wherein the disorder mediated by cell proliferation is selected from the group consisting of cancer, hypertension, hypotension, ischemia, inflammation, arthritis, diabetic retinopathy, myocardial infarction, and cardiovascular disease.

7. The method according to claim 6, wherein the disorder mediated by cell proliferation is cancer.

8. The method according to claim 7, wherein the cancer is selected from the group consisting of breast cancer, colon cancer, central nervous system cancer, leukemia, melanoma, lung cancer, ovarian cancer, prostate cancer, and renal cancer.

9. The method according to claim 8, wherein the cancer is breast cancer.

10. The method according to claim 1, wherein said administering is carried out orally, parenterally, subcutaneously, intravenously, intramuscularly, intraperitoneally, by intravesical instillation, by intracavitary, intravesical instillation, intraocularly, intraarterially, intralesionally, or by application to mucous membrane.

11. A method of regulating activity of a kinase comprising:
contacting the kinase with a polypeptide or protein having an amino acid sequence comprising SEQ ID NO:3, SEQ ID NO:5, or SEQ ID NO:7 under conditions effective to regulate the activity of the kinase.

12. The method according to claim 11, wherein the kinase is a cytoplasmic tyrosine kinase.

13. The method according to claim 12, wherein the kinase comprises a focal adhesion kinase.

14. The method according to claim 11, wherein said contacting comprises:
inhibiting the kinase.

15. The method according to claim 14, wherein said inhibiting is carried out under conditions effective to regulate integrin-mediated adhesion of a cell of a biological organism.

16. The method according to claim 14, wherein said inhibiting is carried out under conditions effective to regulate spreading of a cell.

17. The method according to claim 14, wherein said inhibiting is carried out under conditions effective to regulate migration of a cell.

18. The method according to claim 14, wherein said inhibiting is carried out under conditions effective to regulate cell cycle progression.

19. The method according to claim 14, wherein said inhibiting is carried out under conditions effective to regulate proliferation of a cell.

20. The method according to claim 14, wherein said inhibiting is carried out by contacting the kinase with a polypeptide or protein comprising an amino acid sequence of SEQ ID NO:3.

21. The method according to claim 20, wherein said inhibiting is carried out under conditions effective to inhibit phosphorylation of cell adhesion-dependent paxillin.

22. The method according to claim 20, wherein said inhibiting is carried out under conditions effective to inhibit phosphorylation of squalene-hopene cyclase.

23. The method according to claim 20, wherein said inhibiting is carried out under conditions effective to inhibit phosphorylation of a protein having a Crk-associated substrate.

24. The method according to claim 20, wherein said inhibiting is carried out under conditions effective to inhibit phosphorylation of growth factor receptor-bound protein 7.

25. The method according to claim 14, wherein said inhibiting is carried out by contacting the kinase with a polypeptide or protein comprising an amino acid sequence of SEQ ID NO:5.

26. The method according to claim 25, wherein said inhibiting is carried out under conditions effective to inhibit phosphorylation of cell adhesion-dependent paxillin.

27. The method according to claim 25, wherein said inhibiting is carried out under conditions effective to inhibit phosphorylation of squalene-hopene cyclase.

28. The method according to claim 25, wherein said inhibiting is carried out under conditions effective to inhibit phosphorylation of a protein having a Crk-associated substrate.

29. The method according to claim 25, wherein said inhibiting is carried out under conditions effective to inhibit phosphorylation of growth factor receptor-bound protein 7.

30 The method according to claim 14, wherein said inhibiting is carried out by contacting the kinase with a polypeptide or protein comprising an amino acid sequence of SEQ ID NO:7.

31. The method according to claim 30, wherein said inhibiting is carried out under conditions effective to inhibit phosphorylation of cell adhesion-dependent paxillin.

32. The method according to claim 30, wherein said inhibiting is carried out under conditions effective to inhibit phosphorylation of squalene-hopene cyclase.

33. The method according to claim 30, wherein said inhibiting is carried out under conditions effective to inhibit phosphorylation of a protein having a Crk-associated substrate.

34. The method according to claim 30, wherein said inhibiting is carried out under conditions effective to inhibit phosphorylation of growth factor receptor-bound protein 7.

35. An expression vector comprising transcriptional and translational regulatory nucleotide sequences operably linked to a nucleic acid molecule having a nucleotide sequence comprising SEQ ID NO:4, SEQ ID NO:6, or SEQ ID NO:8.

36. The expression vector according to claim 35, wherein said nucleic acid molecule comprises the nucleotide sequence of SEQ ID NO:4.

37. The expression vector according to claim 35, wherein said nucleic acid molecule comprises the nucleotide sequence of SEQ ID NO:6.

38. The expression vector according to claim 35, wherein said nucleic acid molecule comprises the nucleotide sequence of SEQ ID NO:8.

39. The expression vector according to claim 35, wherein said nucleic acid molecule is in proper sense orientation and correct reading frame.

40. The expression vector according to claim 35, wherein said vector is an adenoviral vector.

41. The expression vector according to claim 35, wherein said vector is a retroviral vector.

42. The expression vector according to claim 35, wherein said vector is constructed so that said nucleic acid molecule is inducibly expressed.

43. A method of regulating G1 to S phase progression of a cell, said method comprising:
contacting the cell with a polypeptide or protein having an amino acid sequence comprising SEQ ID NO:3, SEQ ID NO:5, or SEQ ID NO:7 under conditions effective to regulate G1 to S phase progression of the cell.

44. The method according to claim 43, wherein the cell is a human cancer cell.

45. The method according to claim 43, wherein the cell is a non-tumorigenic mammary epithelial cell.

46. The method according to claim 43, wherein said protein or polypeptide comprises an amino acid sequence of SEQ ID NO:3.

47. The method according to claim 43, wherein said protein or polypeptide comprises an amino acid sequence of SEQ ID NO:5.

48. The method according to claim 43, wherein said protein or polypeptide comprises an amino acid sequence of SEQ ID NO:7.

49. A method of regulating expression of p21 in a cell, said method comprising:
contacting the cell with a polypeptide or protein having an amino acid sequence comprising SEQ ID NO:3, SEQ ID NO:5, or SEQ ID NO:7 under conditions effective to regulate expression of p21.

50. The method according to claim 49, wherein said cell is a human cancer cell.

51. The method according to claim 49, wherein said protein or polypeptide comprises an amino acid sequence of SEQ ID NO:3.

52. The method according to claim 49, wherein said protein or polypeptide comprises an amino acid sequence of SEQ ID NO:5.

53. The method according to claim 49, wherein said protein or polypeptide comprises an amino acid sequence of SEQ ID NO:7.

54. A method of regulating phosphorylation of retinoblastoma protein in a cell, said method comprising:
contacting the cell with a polypeptide or protein having an amino acid sequence comprising SEQ ID NO:3, SEQ ID NO:5, or SEQ ID NO:7 under conditions effective to regulate phosphorylation of retinoblastoma protein.

55. The method according to claim 54, wherein said cell is a human cancer cell.

56. The method according to claim 54, wherein said protein or polypeptide comprises an amino acid sequence of SEQ ID NO:3.

57. The method according to claim 54, wherein said protein or polypeptide comprises an amino acid sequence of SEQ ID NO:5.

58. The method according to claim 54, wherein said protein or polypeptide comprises an amino acid sequence of SEQ ID NO:7.

59. A method of regulating retinoblastoma protein/E2F transcription factor 1 (GENBANK ACCESSION NO. NP00_{—}5216) complex formation in a cell, said method comprising:
contacting the cell with a polypeptide or protein having an amino acid sequence comprising SEQ ID NO:3, SEQ ID NO:5, or SEQ ID NO:7 under conditions effective to regulate retinoblastoma protein/E2F transcription factor 1 complex formation.

60. The method according to claim 59, wherein said cell is a human cancer cell.

61. The method according to claim 59, wherein said protein or polypeptide comprises an amino acid sequence of SEQ ID NO:3.

62. The method according to claim 59, wherein said protein or polypeptide comprises an amino acid sequence of SEQ ID NO:5.

63. The method according to claim 59, wherein said protein or polypeptide comprises an amino acid sequence of SEQ ID NO:7.

64. A method of regulating detachment-induced apoptosis of a cell, said method comprising:
contacting the cell with a polypeptide or protein having an amino acid sequence comprising SEQ ID NO:3, SEQ ID NO:5, or SEQ ID NO:7 under conditions effective to regulate detachment-induced apoptosis of the cell.

65. The method according to claim 64, wherein said cell is a human cancer cell.

66. The method according to claim 64, wherein said protein or polypeptide comprises an amino acid sequence of SEQ ID NO:3.

67. The method according to claim 64, wherein said protein or polypeptide comprises an amino acid sequence of SEQ ID NO:5.

68. The method according to claim 64, wherein said protein or polypeptide comprises an amino acid sequence of SEQ ID NO:7.

69. A method of regulating anchorage-independent growth of a cell, said method comprising:
contacting the cell with a polypeptide or protein having an amino acid sequence comprising SEQ ID NO:3, SEQ ID NO:5, or SEQ ID NO:7 under conditions effective to regulate anchorage-independent growth of the cell.

70. The method according to claim 69, wherein said cell is a human cancer cell.

71. The method according to claim 69, wherein said protein or polypeptide comprises an amino acid sequence of SEQ ID NO:3.

72. The method according to claim 69, wherein said protein or polypeptide comprises an amino acid sequence of SEQ ID NO:5.

73. The method according to claim 69, wherein said protein or polypeptide comprises an amino acid sequence of SEQ ID NO:7.

74. A method of regulating tumor formation in a subject, said method comprising:
administering a therapeutically effective amount of a protein or polypeptide having an amino acid sequence comprising SEQ ID NO:3, SEQ ID NO:5, or SEQ ID NO:7 to the subject under conditions effective to regulate tumor formation.

75. The method according to claim 74, wherein said subject is a human.

76. The method according to claim 74, wherein said tumor is a breast cancer tumor.

77. The method according to claim 74, wherein said protein or polypeptide comprises an amino acid sequence of SEQ ID NO:3.

78. The method according to claim 74, wherein said protein or polypeptide comprises an amino acid sequence of SEQ ID NO:5.

79. The method according to claim 74, wherein said protein or polypeptide comprises an amino acid sequence of SEQ ID NO:7.

80. A method of regulating tumor growth in a subject, said method comprising:
administering a therapeutically effective amount of a protein or polypeptide having an amino acid sequence comprising SEQ ID NO:3, SEQ ID NO:5, or SEQ ID NO:7 to the subject under conditions effective to regulate tumor growth.

81. The method according to claim 80, wherein said subject is a human.

82. The method according to claim 80, wherein said tumor is a breast cancer tumor.

83. The method according to claim 80, wherein said protein or polypeptide comprises an amino acid sequence of SEQ ID NO:3.

84. The method according to claim 80, wherein said protein or polypeptide comprises an amino acid sequence of SEQ ID NO:5.

85. The method according to claim 80, wherein said protein or polypeptide comprises an amino acid sequence of SEQ ID NO:7.

86. A method of regulating tumor formation in a subject, said method comprising:
administering a therapeutically effective amount of a nucleic acid molecule having a nucleotide sequence comprising SEQ ID NO:4, SEQ ID NO:6, or SEQ ID NO:8 to the subject under conditions effective to regulate tumor formation.

87. The method according to claim 86, wherein said subject is a human.

88. The method according to claim 86, wherein said tumor is a breast cancer tumor.

89. The method according to claim 86, wherein said nucleic acid molecule comprises a nucleotide sequence of SEQ ID NO:4.

90. The method according to claim 86, wherein said nucleic acid molecule comprises a nucleotide sequence of SEQ ID NO:6.

91. The method according to claim 86, wherein said nucleic acid molecule comprises a nucleotide sequence of SEQ ID NO:8.

92. A method of regulating tumor growth in a subject, said method comprising:
administering a therapeutically effective amount of a nucleic acid molecule having a nucleotide sequence comprising SEQ ID NO:4, SEQ ID NO:6, or SEQ ID NO:8 to the subject under conditions effective to regulate tumor growth.

93. The method according to claim 92, wherein said subject is a human.

94. The method according to claim 92, wherein said tumor is a breast cancer tumor.

95. The method according to claim 92, wherein said nucleic acid molecule comprises a nucleotide sequence of SEQ ID NO:4.

96. The method according to claim 92, wherein said nucleic acid molecule comprises a nucleotide sequence of SEQ ID NO:6.

97. The method according to claim 92, wherein said nucleic acid molecule comprises a nucleotide sequence of SEQ ID NO:8.


SEQUENCE LISTING (14 sequences)

SEQ ID NO: 1 - protein, 1591 aa, Homo sapiens
MKLYVFLVNTGTTLTFDTELTVQTVADLKHAIQSKYKIAIQHQVLVVNGGECMAADRRVCTYSAGTDTNPIFLFNKEMILCDRPPAIPKTTFSTENDMEIKVEESLMMPAVFHTVASRTQLALEMYEVAKKLCSFCEGLVHDEHLQHQGWAAIMANLEDCSNSYQKLLFKFESIYSNYLQSIEDIKLKLTHLGTAVSVMAKIPLLECLTRHSYRECLGRLDSLPEHEDSEKAETKRSTELVLSPDMPRTTNESLLTSFPKSVEHVSPDTADAESGKEIRESCQSTVHQQDETTIDTKDGDLPFFNVSLLDWINVQDRPNDVESLVRKCFDSMSRLDPRIIRPFIAECRQTIAKLDNQNMKAIKGLEDRLYALDQMIASCGRLVNEQKELAQGFLANQKRAENLKDASVLPDLCLSHANQLMIMLQNHRKLLDIKQKCTTAKQELANNLHVRLKWCCFVMLHADQDGEKLQALLRLVIELLERVKIVEALSTVPQMYCLAVVEVVRRKMFIKHYREWAGALVKDGKRLYEAEKSKRESFGKLFRKSFLRNRLFRGLDSWPPSFCTQKPRKFDCELPDISLKDLQFLQSFCPSEVQPFLRVPLLCDFEPLHQHVLALHNLVKAAQSLDEMSQTITDLLSEQKASVSQTSPQSASSPRMESTAGITTTTSPRTPPPLTVQDPLCPAVCPLEELSPDSIDAHTFDFETIPHPNIEQTIHQVSLDLDSLAESPESDFMSAVNEFVIEENLSSPNPISDPQSPEMMVESLYSSVINAIDSRRMQDTNVCGKEDFGDHTSLNVQLERCRVVAQDSHFSIQTIKEDLCHFRTFVQKEQCDFSNSLKCTAVEIRNIIEKVKCSLEITLKEKHQKELLSLKNEYEGKLDGLIKETEENENKIKKLKGELVCLEEVLQNKDNEFALVKHEKEAVICLQNEKDQKLLEMENIMHSQNCEIKELKQSREIVLEDLKKLHVENDEKLQLLRAELQSLEQSHLKELEDTLQVRHIQEFEKVMTDHRVSLEELKKENQQIINQIQESHAEIIQEKEKQLQELKLKVSDLSDTRCKLEVELALKEAETDEIKILLEESRAQQKETLKSLLEQETENLRTEISKLNQKIQDNNENYQVGLAELRTLMTIEKDQCISELISRHEEESNILKAELNKVTSLHNQAFEIEKNLKEQIIELQSKLDSELSALERQKDEKITQQEEKYEAIIQNLEKDRQKLVSSQEQDREQLIQKLNCEKDEAIQTALKEFKLEREVVEKELLEKVKHLENQIAKSPAIDSTRGDSSSLVAELQEKLQEEKAKFLEQLEEQEKRKNEEMQNVRTSLIAEQQTNFNTVLTREKMRKENIINDLSDKLKSTMQQQERDKDLIESLSEDRARLLEEKKKLEEEVSKLRSSSFVPSPYVATAPELYGACAPELPGESDRSAVETADEGRVDSAMETSMMSVQENIHMLSEEKQRIMLLERTLQLKEEENKRLNQRLMSQSMSSVSSRHSEKIAIRDFQVGDLVLIILDERHDNYVLFTVSPTLYFLHSESLPALDLKPASGASRRPWVLGKVMEKEYCQAKKAQNRFKVPLGTKFYRVKAVSWNKKV

SEQ ID NO: 2 - DNA, 4776 bp, Homo sapiens
atgaagttatatgtatttctggttaacactggaactactctaacatttgacactgaacttacagtgcaaactgtggcagaccttaagcatgccattcaaagcaaatacaagattgctattcaacaccaggtgctggtggtcaatggaggagaatgcatggctgcagatcgaagagtgtgtacctacagtgctgggacggatacaaatccaatttttctttttaacaaagaaatgatcttatgtgatcgtccacctgctattcctaaaactaccttttcgacagaaaatgacatggaaataaaagttgaagaatctcttatgatgcctgcagtttttcatactgttgcttcaaggacacagcttgcattggaaatgtatgaagttgccaagaaactttgttctttttgtgaaggtcttgtacatgatgaacatcttcaacaccaaggctgggctgcaatcatggccaacctggaggactgttcaaattcataccaaaagctacttttcaagtttgaaagtatttattcaaattatctgcagtccatagaagacatcaagttaaaacttactcatttaggaactgcagtttcagtaatggccaagattccactgttggagtgcctaaccagacatagttacagagaatgtttgggaagactggattctttacctgaacatgaagactcagaaaaagctgagacgaaaagatccactgaactggtgctctctcctgatatgcctagaacaactaacgaatctttgttaacctcatttcccaagtcagtggaacatgtgtccccagataccgcagatgctgaaagtggcaaagaaattagggaatcttgtcaaagtactgttcatcagcaagatgaaactacgattgacactaaagatggtgatctgcccttttttaatgtctctttgttagactggataaatgttcaagatagacctaatgatgtggaatctttggtcaggaagtgctttgattctatgagcaggcttgatccaaggattattcgaccatttatagcagaatgccgtcaaactattgccaaacttgataatcagaatatgaaagccattaaaggacttgaagatcggctctacgccctggaccagatgattgctagctgtggccgactggtgaatgaacagaaagagcttgctcagggatttttagctaatcagaagagagctgaaaacttaaaggatgcatctgtattacctgatttatgcctgagtcacgcaaatcagttgatgattatgttgcaaaatcatagaaaactgttagatattaagcagaagtgtaccactgccaaacaagaactagcaaataacctacatgtcagactgaagtggtgttgctttgtaatgcttcatgctgatcaagatggagagaagttacaagctttgctccgcctcgtaatagagctgttagaaagagtcaaaattgttgaagctcttagtacagttcctcagatgtactgcttagctgttgttgaggttgtaagaagaaaaatgttcataaaacactacagggagtgggctggtgctttagtcaaagatggaaagagattatatgaagcagaaaaatcaaaaagggaatcctttgggaaattatttaggaagtcttttttaagaaatcgtctgtttaggggactggactcctggcccccttccttttgtactcaaaagcctcgaaagtttgactgtgaacttccagatatttcattaaaagatttacagtttctgcaatcattttgtccttcggaagttcagccattcctcagggttcccttactttgtgactttgaacctctacaccagcatgtacttgctctacataatttggtaaaagcagcacaaagtttggatgaaatgtcacagaccattacagatctactgagtgaacaaaaggcatctgtgagtcagacatccccacagtctgcttcttcaccaaggatggaaagtacagcaggaattacaactactacctcaccgagaactcctccaccactgactgttcaggatcccttatgtcctgcagtttgtcccttagaagaattatctccagatagtattgatgcacatacgtttgattttgaaactattccccatccaaacatagaacagactattcaccaagtttctttagacttggattcattagcagaaagtcctgaatcagattttatgtctgctgtgaatgagtttgtaatagaagaaaatttgtcgtctcctaatcctataagtgatccacaaagcccagaaatgatggtggaatcactttattcatcagttatcaatgcgatagacagtagacgaatgcaggatacaaatgtatgtggtaaggaggattttggagatcatacttctctgaatgtccagttggaaagatgtagagttgttgcccaagactctcacttcagtatacaaaccattaaggaagacctttgccactttagaacatttgtacaaaaagaacagtgtgacttctcaaattcattaaaatgtacagcagtagaaataagaaacattattgaaaaagtaaaatgttctctggaaataacactaaaagaaaaacatcaaaaagaactactgtctttaaaaaatgaatatgaaggtaaacttgacggactaataaaggaaactgaagagaatgaaaacaaaattaaaaaattgaagggagagttagtatgccttgaggaggttttacaaaataaagataatgaatttgctttggttaaacatgaaaaagaagctgtaatctgcctgcagaatgaaaaggatcagaagttgttagagatggaaaatataatgcactctcaaaattgtgaaattaaagaactgaagcagtcacgagaaatagtgttagaagacttaaaaaagctccatgttgaaaatgatgagaagttacagttattgagggcagaacttcagtccttggagcaaagtcatctaaaggaattagaggacacacttcaggttaggcacatacaagagtttgagaaggttatgacagaccacagagtttctttggaggaattaaaaaaggaaaaccaacaaataattaatcaaatacaagaatctcatgctgaaattatccaggaaaaagaaaaacagttacaggaattaaaactcaaggtttctgatttgtcagacacgagatgcaagttagaggttgaacttgcgttgaaggaagcagaaactgatgaaataaaaattttgctggaagaaagcagagcccagcagaaggagaccttgaaatctcttcttgaacaagagacagaaaatttgagaacagaaattagtaaactcaaccaaaagattcaggataataatgaaaattatcaggtgggcttagcagagctaagaactttaatgacaattgaaaaagatcagtgtatttccgagttaattagtagacatgaagaagaatctaatatacttaaagctgaattaaacaaagtaacatctttgcataaccaagcatttgaaatagaaaaaaacctaaaagaacaaataattgaactgcagagtaaattggattcagaattgagtgctcttgaaagacaaaaagatgaaaaaattacccaacaagaagagaaatacgaagctattatccagaaccttgagaaagacagacaaaaattggtcagcagccaggagcaagacagagaacagttaattcagaagcttaattgtgaaaaagatgaagctattcagactgccctaaaagaatttaaattggagagagaagttgttgagaaagagttattagaaaaagttaaacatcttgagaatcaaatagcaaaaagtcctgccattgactctaccagaggagattcttcaagcttagttgctgaacttcaagaaaagcttcaggaagaaaaagctaagtttctagaacaacttgaagagcaagaaaaaagaaagaatgaagaaatgcaaaatgttcgaacatctttgattgcggaacaacagaccaattttaacactgttttaacaagagagaaaatgagaaaagaaaacataataaatgatcttagtgataagttgaaaagtacaatgcagcaacaagaacgggataaagatttgatagagtcactttctgaagatcgagctcgtttgcttgaggaaaagaaaaagcttgaagaagaagtcagtaagttgcgcagtagcagttttgttccttcaccatatgtagctacagccccagaactttatggagcttgtgcacctgaactcccaggtgaatcagatagatccgctgtggaaacagcagatgaaggaagagtggattcagcaatggagacaagcatgatgtctgtacaagaaaatattcatatgttgtctgaagaaaaacagcggataatgctgttagaacgaacattgcaattgaaagaagaagaaaataaacggttaaatcaaagactgatgtctcagagcatgtcttcagtatcttcaaggcattctgaaaagatagctattagagattttcaggtgggagatttggtactcatcatcctagacgaacgccatgacaattatgtgttatttactgttagtcctactttatattttctacattcagagtctctacctgccctggatctcaaaccagcttcaggtgcatctagaagaccctgggtactcggaaaagtaatggaaaaagaatactgtcaagccaaaaaggcacaaaacagatttaaagttcctttggggacaaagttttacagagtgaaagccgtatcatggaataagaaagtataa

SEQ ID NO: 3 - protein, 638 aa, Homo sapiens
MKLYVFLVNTGTTLTFDTELTVQTVADLKHAIQSKYKIAIQHQVLVVNGGECMAADRRVCTYSAGTDTNPIFLFNKEMILCDRPPAIPKTTFSTENDMEIKVEESLMMPAVFHTVASRTQLALEMYEVAKKLCSFCEGLVHDEHLQHQGWAAIMANLEDCSNSYQKLLFKFESIYSNYLQSIEDIKLKLTHLGTAVSVMAKIPLLECLTRHSYRECLGRLDSLPEHEDSEKAETKRSTELVLSPDMPRTTNESLLTSFPKSVEHVSPDTADAESGKEIRESCQSTVHQQDETTIDTKDGDLPFFNVSLLDWINVQDRPNDVESLVRKCFDSMSRLDPRIIRPFIAECRQTIAKLDNQNMKAIKGLEDRLYALDQMIASCGRLVNEQKELAQGFLANQKRAENLKDASVLPDLCLSHANQLMIMLQNHRKLLDIKQKCTTAKQELANNLHVRLKWCCFVMLHADQDGEKLQALLRLVIELLERVKIVEALSTVPQMYCLAVVEVVRRKMFIKHYREWAGALVKDGKRLYEAEKSKRESFGKLFRKSFLRNRLFRGLDSWPPSFCTQKPRKFDCELPDISLKDLQFLQSFCPSEVQPFLRVPLLCDFEPLHQHVLALHNLVKAAQSLDEMSQTITDLLSE

SEQ ID NO: 4 - DNA, 1914 bp, Homo sapiens
atgaagttatatgtatttctggttaacactggaactactctaacatttgacactgaacttacagtgcaaactgtggcagaccttaagcatgccattcaaagcaaatacaagattgctattcaacaccaggtgctggtggtcaatggaggagaatgcatggctgcagatcgaagagtgtgtacctacagtgctgggacggatacaaatccaatttttctttttaacaaagaaatgatcttatgtgatcgtccacctgctattcctaaaactaccttttcgacagaaaatgacatggaaataaaagttgaagaatctcttatgatgcctgcagtttttcatactgttgcttcaaggacacagcttgcattggaaatgtatgaagttgccaagaaactttgttctttttgtgaaggtcttgtacatgatgaacatcttcaacaccaaggctgggctgcaatcatggccaacctggaggactgttcaaattcataccaaaagctacttttcaagtttgaaagtatttattcaaattatctgcagtccatagaagacatcaagttaaaacttactcatttaggaactgcagtttcagtaatggccaagattccactgttggagtgcctaaccagacatagttacagagaatgtttgggaagactggattctttacctgaacatgaagactcagaaaaagctgagacgaaaagatccactgaactggtgctctctcctgatatgcctagaacaactaacgaatctttgttaacctcatttcccaagtcagtggaacatgtgtccccagataccgcagatgctgaaagtggcaaagaaattagggaatcttgtcaaagtactgttcatcagcaagatgaaactacgattgacactaaagatggtgatctgcccttttttaatgtctctttgttagactggataaatgttcaagatagacctaatgatgtggaatctttggtcaggaagtgctttgattctatgagcaggcttgatccaaggattattcgaccatttatagcagaatgccgtcaaactattgccaaacttgataatcagaatatgaaagccattaaaggacttgaagatcggctctacgccctggaccagatgattgctagctgtggccgactggtgaatgaacagaaagagcttgctcagggatttttagctaatcagaagagagctgaaaacttaaaggatgcatctgtattacctgatttatgcctgagtcacgcaaatcagttgatgattatgttgcaaaatcatagaaaactgttagatattaagcagaagtgtaccactgccaaacaagaactagcaaataacctacatgtcagactgaagtggtgttgctttgtaatgcttcatgctgatcaagatggagagaagttacaagctttgctccgcctcgtaatagagctgttagaaagagtcaaaattgttgaagctcttagtacagttcctcagatgtactgcttagctgttgttgaggttgtaagaagaaaaatgttcataaaacactacagggagtgggctggtgctttagtcaaagatggaaagagattatatgaagcagaaaaatcaaaaagggaatcctttgggaaattatttaggaagtcttttttaagaaatcgtctgtttaggggactggactcctggcccccttccttttgtactcaaaagcctcgaaagtttgactgtgaacttccagatatttcattaaaagatttacagtttctgcaatcattttgtccttcggaagttcagccattcctcagggttcccttactttgtgactttgaacctctacaccagcatgtacttgctctacataatttggtaaaagcagcacaaagtttggatgaaatgtcacagaccattacagatctactgagtgaa

SEQ ID NO: 5 - protein, 154 aa, Homo sapiens
MKLYVFLVNTGTTLTFDTELTVQTVADLKHAIQSKYKIAIQHQVLVVNGGECMAADRRVCTYSAGTDTNPIFLFNKEMILCDRPPAIPKTTFSTENDMEIKVEESLMMPAVFHTVASRTQLALEMYEVAKKLCSFCEGLVHDEHLQHQGWAAIM

SEQ ID NO: 6 - DNA, 462 bp, Homo sapiens
atgaagttatatgtatttctggttaacactggaactactctaacatttgacactgaacttacagtgcaaactgtggcagaccttaagcatgccattcaaagcaaatacaagattgctattcaacaccaggtgctggtggtcaatggaggagaatgcatggctgcagatcgaagagtgtgtacctacagtgctgggacggatacaaatccaatttttctttttaacaaagaaatgatcttatgtgatcgtccacctgctattcctaaaactaccttttcgacagaaaatgacatggaaataaaagttgaagaatctcttatgatgcctgcagtttttcatactgttgcttcaaggacacagcttgcattggaaatgtatgaagttgccaagaaactttgttctttttgtgaaggtcttgtacatgatgaacatcttcaacaccaaggctgggctgcaatcatg

SEQ ID NO: 7 - protein, 735 aa, Homo sapiens
QKASVSQTSPQSASSPRMESTAGITTTTSPRTPPPLTVQDPLCPAVCPLEELSPDSIDAHTFDFETIPHPNIEQTIHQVSLDLDSLAESPESDFMSAVNEFVIEENLSSPNPISDPQSPEMMVESLYSSVINAIDSRRMQDTNVCGKEDFGDHTSLNVQLERCRVVAQDSHFSIQTIKEDLCHFRTFVQKEQCDFSNSLKCTAVEIRNIIEKVKCSLEITLKEKHQKELLSLKNEYEGKLDGLIKETEENENKIKKLKGELVCLEEVLQNKDNEFALVKHEKEAVICLQNEKDQKLLEMENIMHSQNCEIKELKQSREIVLEDLKKLHVENDEKLQLLRAELQSLEQSHLKELEDTLQVRHIQEFEKVMTDHRVSLEELKKENQQIINQIQESHAEIIQEKEKQLQELKLKVSDLSDTRCKLEVELALKEAETDEIKILLEESRAQQKETLKSLLEQETENLRTEISKLNQKIQDNNENYQVGLAELRTLMTIEKDQCISELISRHEEESNILKAELNKVTSLHNQAFEIEKNLKEQIIELQSKLDSELSALERQKDEKITQQEEKYEAIIQNLEKDRQKLVSSQEQDREQLIQKLNCEKDEAIQTALKEFKLEREVVEKELLEKVKHLENQIAKSPAIDSTRGDSSSLVAELQEKLQEEKAKFLEQLEEQEKRKNEEMQNVRTSLIAEQQTNFNTVLTREKMRKENIINDLSDKLKSTMQQQERDKDLIESLSE

SEQ ID NO: 8 - DNA, 2205 bp, Homo sapiens
caaaaggcatctgtgagtcagacatccccacagtctgcttcttcaccaaggatggaaagtacagcaggaattacaactactacctcaccgagaactcctccaccactgactgttcaggatcccttatgtcctgcagtttgtcccttagaagaattatctccagatagtattgatgcacatacgtttgattttgaaactattccccatccaaacatagaacagactattcaccaagtttctttagacttggattcattagcagaaagtcctgaatcagattttatgtctgctgtgaatgagtttgtaatagaagaaaatttgtcgtctcctaatcctataagtgatccacaaagcccagaaatgatggtggaatcactttattcatcagttatcaatgcgatagacagtagacgaatgcaggatacaaatgtatgtggtaaggaggattttggagatcatacttctctgaatgtccagttggaaagatgtagagttgttgcccaagactctcacttcagtatacaaaccattaaggaagacctttgccactttagaacatttgtacaaaaagaacagtgtgacttctcaaattcattaaaatgtacagcagtagaaataagaaacattattgaaaaagtaaaatgttctctggaaataacactaaaagaaaaacatcaaaaagaactactgtctttaaaaaatgaatatgaaggtaaacttgacggactaataaaggaaactgaagagaatgaaaacaaaattaaaaaattgaagggagagttagtatgccttgaggaggttttacaaaataaagataatgaatttgctttggttaaacatgaaaaagaagctgtaatctgcctgcagaatgaaaaggatcagaagttgttagagatggaaaatataatgcactctcaaaattgtgaaattaaagaactgaagcagtcacgagaaatagtgttagaagacttaaaaaagctccatgttgaaaatgatgagaagttacagttattgagggcagaacttcagtccttggagcaaagtcatctaaaggaattagaggacacacttcaggttaggcacatacaagagtttgagaaggttatgacagaccacagagtttctttggaggaattaaaaaaggaaaaccaacaaataattaatcaaatacaagaatctcatgctgaaattatccaggaaaaagaaaaacagttacaggaattaaaactcaaggtttctgatttgtcagacacgagatgcaagttagaggttgaacttgcgttgaaggaagcagaaactgatgaaataaaaattttgctggaagaaagcagagcccagcagaaggagaccttgaaatctcttcttgaacaagagacagaaaatttgagaacagaaattagtaaactcaaccaaaagattcaggataataatgaaaattatcaggtgggcttagcagagctaagaactttaatgacaattgaaaaagatcagtgtatttccgagttaattagtagacatgaagaagaatctaatatacttaaagctgaattaaacaaagtaacatctttgcataaccaagcatttgaaatagaaaaaaacctaaaagaacaaataattgaactgcagagtaaattggattcagaattgagtgctcttgaaagacaaaaagatgaaaaaattacccaacaagaagagaaatacgaagctattatccagaaccttgagaaagacagacaaaaattggtcagcagccaggagcaagacagagaacagttaattcagaagcttaattgtgaaaaagatgaagctattcagactgccctaaaagaatttaaattggagagagaagttgttgagaaagagttattagaaaaagttaaacatcttgagaatcaaatagcaaaaagtcctgccattgactctaccagaggagattcttcaagcttagttgctgaacttcaagaaaagcttcaggaagaaaaagctaagtttctagaacaacttgaagagcaagaaaaaagaaagaatgaagaaatgcaaaatgttcgaacatctttgattgcggaacaacagaccaattttaacactgttttaacaagagagaaaatgagaaaagaaaacataataaatgatcttagtgataagttgaaaagtacaatgcagcaacaagaacgggataaagatttgatagagtcactttctgaa

SEQ ID NO: 9 - DNA, 24 bp, Artificial Sequence
  Description of Artificial Sequence oligonucleotide primer
ctggatccatggcagcttaccttg

SEQ ID NO: 10 - DNA, 26 bp, Artificial Sequence
  Description of Artificial Sequence oligonucleotide primer
atgatatcttaagtatcttcttcatc

SEQ ID NO: 11 - DNA, 28 bp, Artificial Sequence
  Description of Artificial Sequence oligonucleotide primer
atgatatcaaccagagattatgaaattc

SEQ ID NO: 12 - DNA, 30 bp, Artificial Sequence
  Description of Artificial Sequence oligonucleotide primer
gcttaaattaagtaaacctgggtcgtctac

SEQ ID NO: 13 - DNA, 28 bp, Artificial Sequence
  Description of Artificial Sequence oligonucleotide primer
ccaggatccggcattgcccgtgaagatg

SEQ ID NO: 14 - DNA, 27 bp, Artificial Sequence
  Description of Artificial Sequence oligonucleotide primer
atgaattcgcttcacaccagctcggtg